SEQUENCE LISTING (14 sequences)

SEQ ID NO: 1 - DNA, 2259 bp, Homo sapiens
atgcttgtcgccggtcttcttctctgggcttccctactgactggggcctggccatccttccctacccaggaccacctcccggccacgccccgggtacggctctcattcaaagagctgaaggccacaggcaccgcccacttcttcaacttcctgctcaacacaaccgactaccgaatcttgctcaaggacgaggaccacgaccgcatgtacgtgggcagcaaggactacgtgctgtccctggacctgcacgacatcaaccgcgagcccctcattatacactgggcagcctccccacagcgcatcgaggaatgcgtgctctcaggcaaggatgtcaacggcgagtgtgggaacttcgtcaggctcatccagccctggaaccgaacacacctgtatgtgtgcgggacaggtgcctacaaccccatgtgcacctatgtgaaccgcggacgccgcgcccaggattacatcttctacctggagcctgagcgactcgagtcagggaagggcaagtgtccgtacgatcccaagctggacacagcatcggccctcatcaatgaggagctctatgctggtgtgtacatcgattttatgggcactgatgcagccatcttccgcacacttggaaagcagacagccatgcgcacggatcagtacaactcccggtggctgaacgacccgtcgttcatccatgctgagctcattcctgacagtgcggagaatgatgataagctttacttcttcttccgtgagcggtcggcagaggcgccgcagagccccgcggtgtacgcccgcatcgggcgcatttgcctgaacgatgacggtggtcactgttgcctggtcaacaagtggagcacattcctgaaggcgcggctcgtctgctctgtcccgggcgaggatggcattgagactcactttgatgagctccaggacgtgtttgtccagcagacccaggacgtgaggaaccctgtcatttacgctgtctttacctcctctggctccgtgttccgaggctctgccgtgtgtgtctactccatggctgatattcgcatggtcttcaacgggccctttgcccacaaagaggggcccaactaccagtggatgcccttctcagggaagatgccctacccacggccgggcacgtgccctggtggaaccttcacgccatctatgaagtccaccaaggattatcctgatgaggtgatcaacttcatgcgcagccacccactcatgtaccaggccgtgtaccctctgcagcggcggcccctggtagtccgcacaggtgctccctaccgccttaccactattgccgtggaccaggtggattcagccgacgggcgctatgaggtgcttttcctgggcacagaccgcgggacagtgcagaaggtcattgtgctgcccaaggatgaccaggagatggaggagctcatgctggaggaggtggaggtcttcaaggatccagcacccgtcaagaccatgaccatctcttctaagaggcaacaactctacgtggcgtcagccgtgggtgtcacacacctgagcctgcaccgctgccaggcgtatggggctgcctgtgctgactgctgccttgcccgggacccttactgtgcctgggatggccaggcctgctcccgctatacagcatcctccaagaggcggagccgccggcaggacgtccggcacggaaaccccatcaggcagtgccgtgggttcaactccaatgccaacaagaatgccgtggagtctgtgcagtatggcgtggccggcagcgcagccttccttgagtgccagccccgctcgccccaagccactgttaagtggctgttccagcgagatcctggtgaccggcgccgagagattcgtgcagaggaccgcttcctgcgcacagagcagggcttgttgctccgtgcactgcagctcagcgatcgtggcctctactcctgcacagccactgagaacaactttaagcacgtcgtcacacgagtgcagctgcatgtactgggccgggacgccgtccatgctgccctcttcccaccactgtccatgagcgccccgccacccccaggcgcaggccccccaacgcctccttaccaggagttagcccagctgctggcccagccagaagtgggcctcatccaccagtactgccagggttactggcgccatgtgccccccagccccagggaggctccaggggcaccccggtctcctgagccccaggaccagaaaaagccccggaaccgccggcaccaccctccggacaca

SEQ ID NO: 2 - DNA, 2355 bp, Homo sapiens
atgcttgtcgccggtcttcttctctgggcttccctactgaccggggcctggccatccttccccacccaggaccacctcccggccacgccccgggtccggctctcattcaaagagctgaaggccacaggcaccgcccacttcttcaacttcctgctcaacacaaccgactaccgaatcttgctcaaggacgaggaccacgaccgcatgtacgtgggcagcaaggactacgtgctgtccctggacctgcacgacatcaaccgcgagcccctcattatacactgggcagcctccccacagcgcatcgaggaatgcgtgctctcaggcaaggatgtcaacggcgagtgtgggaacttcgtcaggctcatccagccctggaaccgaacacacctgtatgtgtgcgggacaggtgcctacaaccccatgtgcacctatgtgaaccgcggacgccgcgcccaggccacaccatggacccagactcaggcggtcagaggccgcggcagcagagccacggatggtgccctccgcccgatgcccacagccccacgccaggattacatcttctacctggagcctgagcgactcgagtcagggaagggcaagtgtccgtacgatcccaagctggacacagcatcggccctcatcaatgaggagctctatgctggtgtgtacatcgattttatgggcactgatgcagccatcttccgcacacttggaaagcagacagccatgcgcacggatcagtacaactcccggtggctgaacgacccgtcgttcatccatgctgagctcattcctgacagtgcggagcgcaatgatgataagctttacttcttcttccgtgagcggtcggcagaggcgccgcagagccccgcggtgtacgcccgcatcgggcgcatttgcctgaacgatgacggtggtcactgttgcctggtcaacaagtggagcacattcctgaaggcgcggctcgtctgctctgtcccgggcgaggatggcattgagactcactttgatgagctccaggacgtgtttgtccagcagacccaggacgtgaggaaccctgtcatttacgctgtctttacctcctctggctccgtgttccgaggctctgccgtgtgtgtctactccatggctgatattcgcatggtcttcaacgggccctttgcccacaaagaggggcccaactaccagtggatgcccttctcagggaagatgccctacccacggccgggcacgtgccctggtggaaccttcacgccatctatgaagtccaccaaggattatcctgatgaggtgatcaacttcatgcgcagccacccactcatgtaccaggccgtgtaccctctgcagcggcggcccctggtagtccgcacaggtgctccctaccgccttaccactattgccgtggaccaggtggatgcaggcgacgggcgctatgaggtgcttttcctgggcacagaccgcgggacagtgcagaaggtcattgtgctgcccaaggatgaccaggagatggaggagctcatgctggaggaggtggaggtcttcaaggatccagcacccgtcaagaccatgaccatctcttctaagaggcaacaactctacgtggcgtcagccgtgggtgtcacacacctgagcctgcaccgctgccaggcgtatggggctgcctgtgctgactgctgccttgcccgggacccttactgtgcctgggatggccaggcctgctcccgctatacagcatcctccaagaggcggagccgccggcaggacgtccggcacggaaaccccatcaggcagtgccgtgggttcaactccaatgccaacaagaatgccgtggagtctgtgcagtatggcgtggccggcagcgcagccttccttgagtgccagccccgctcgccccaagccactgttaagtggctgttccagcgagatcctggtgaccggcgccgagagattcgtgcagaggaccgcttcctgcgcacagagcagggcttgttgctccgtgcactgcagctcagcgatcgtggcctctactcctgcacagccactgagaacaactttaagcacgtcgtcacacgagtgcagctgcatgtactgggccgggacgccgtccatgctgccctcttcccaccactgtccatgagcgccccgccacccccaggcgcaggccccccaacgcctccttaccaggagttagcccagctgctggcccagccagaagtgggcctcatccaccagtactgccagggttactggcgccatgtgccccccagccccagggaggctccaggggcaccccggtctcctgagccccaggaccagaaaaagccccggaaccgccggcaccaccctccggacaca

SEQ ID NO: 3 - DNA, 2355 bp, Homo sapiens
atgcttgtcgccggtcttcttctctgggcttccctactgaccggggcctggccatccttccccacccaggaccacctcccggccacgccccgggtccggctctcattcaaagagctgaaggccacaggcaccgcccacttcttcaacttcctgctcaacacaaccgactaccgaatcttgctcaaggacgaggaccacgaccgcatgtacgtgggcagcaaggactacgtgctgtccctggacctgcacgacatcaaccgcgagcccctcattatacactgggcagcctccccacagcgcatcgaggaatgcgtgctctcaggcaaggatgtcaacggcgagtgtgggaacttcgtcaggctcatccagccctggaaccgaacacacctgtatgtgtgcgggacaggtgcctacaaccccatgtgcacctatgtgaaccgcggacgccgcgcccaggccacaccatggacccagactcaggcggtcagaggccgcggcagcagagccacggatggtgccctccgcccgatgcccacagccccacgccaggattacatcttctacctggagcctgagcgactcgagtcagggaagggcaagtgtccgtacgatcccaagctggacacagcatcggccctcatcaatgaggagctctatgctggtgtgtacatcgattttatgggcactgatgcagccatcttccgcacacttggaaagcagacagccatgcgcacggatcagtacaactcccggtggctgaacgacccgtcgttcatccatgctgagctcattcctgacagtgcggagcgcaatgatgataagctttacttcttcttccgtgagcggtcggcagaggcgccgcagagccccgcggtgtacgcccgcatcgggcgcatttgcctgaacgatgacggtggtcactgttgcctggtcaacaagtggagcacattcctgaaggcgcggctcgtctgctctgtcccgggcgaggatggcattgagactcactttgatgagctccaggacgtgtttgtccagcagacccaggacgtgaggaaccctgtcatttacgctgtctttacctcctctggctccgtgttccgaggctctgccgtgtgtgtctactccatggctgatattcgcatggtcttcaacgggccctttgcccacaaagaggggcccaactaccagtggatgcccttctcagggaagatgccctacccacggccgggcacgtgccctggtggaaccttcacgccatctatgaagtccaccaaggattatcctgatgaggtgatcaacttcatgcgcagccacccactcatgtaccaggccgtgtaccctctgcagcggcggcccctggtagtccgcacaggtgctccctaccgccttaccactattgccgtggaccaggtggatgcagccgacgggcgctatgaggtgcttttcctgggcacagaccgcgggacagtgcagaaggtcattgtgctgcccaaggatgaccaggagttggaggagctcatgctggaggaggtggaggtcttcaaggatccagcacccgtcaagaccatgaccatctcttctaagaggcaacaactctacgtggcgtcagccgtgggtgtcacacacctgagcctgcaccgctgccaggcgtatggggctgcctgtgctgactgctgccttgcccgggacccttactgtgcctgggatggccaggcctgctcccgctatacagcatcctccaagaggcggagccgccggcaggacgtccggcacggaaaccccatcaggcagtgccgtgggttcaactccaatgccaacaagaatgccgtggagtctgtgcagtatggcgtggccggcagcgcagccttccttgagtgccagccccgctcgccccaagccactgttaagtggctgttccagcgagatcctggtgaccggcgccgagagattcgtgcagaggaccgcttcctgcgcacagagcagggcttgttgctccgtgcactgcagctcagcgatcgtggcctctactcctgcacagccactgagaacaactttaagcacgtcgtcacacgagtgcagctgcatgtactgggccgggacgccgtccatgctgccctcttcccaccactgtccatgagcgccccgccacccccaggcgcaggccccccaacgcctccttaccaggagttagcccagctgctggcccagccagaagtgggcctcatccaccagtactgccagggttactggcgccatgtgccccccagccccagggaggctccaggggcaccccggtctcctgagccccaggaccagaaaaagccccggaaccgccggcaccaccctccggacaca

SEQ ID NO: 4 - DNA, 2355 bp, Homo sapiens
atgcttgtcgccggtcttcttctctgggcttccctactgaccggggcctggccatccttccccacccaggaccacctcccggccacgccccgggtccggctctcattcaaagagctgaaggccacaggcaccgcccacttcttcaacttcctgctcaacacaaccgactaccgaatcttgctcaaggacgaggaccacgaccgcatgtacgtgggcagcaaggactacgtgctgtccctggacctgcacgacatcaaccgcgagcccctcattatacactgggcagcctccccacagcgcatcgaggaatgcgtgctctcaggcaaggatgtcaacggcgagtgtgggaacttcgtcaggctcatccagccctggaaccgaacacacctgtatgtgtgcgggacaggtgcctacaaccccatgtgcacctatgtgaaccgcggacgccgcgcccaggccacaccatggacccagactcaggcggtcagaggccgcggcagcagagccacggatggtgccctccgcccgatgcccacagccccacgccaggattacatcttctacctggagcctgagcgactcgagtcagggaagggcaagtgtccgtacgatcccaagctggacacagcatcggccctcatcaatgaggagctctatgctggtgtgtacatcgattttatgggcactgatgcagccatcttccgcacacttggaaagcagacagccatgcgcacggatcagtacaactcccggtggctgaacgacccgtcgttcatccatgctgagctcattcctgacagtgcggagcgcaatgatgataagctttacttcttcttccgtgagcggtcggcagaggcgccgcagagccccgcggtgtacgcccgcatcgggcgcatttgcctgaacgatgacggtggtcactgttgcctggtcaacaagtggagcacattcctgaaggcgcggctcgtctgctctgtcccgggcgaggatggcattgagactcactttgatgagctccaggacgtgtttgtccagcagacccaggacgtgaggaaccctgtcatttacgctgtctttacctcctctggctccgtgttccgaggctctgccgtgtgtgtctactccatggctgatattcgcatggtcttcaacgggccctttgcccacaaagaggggcccaactaccagtggatgcccttctcagggaagatgccctacccacggccgggcacgtgccctggtggaaccttcacgccatctatgaagtccaccaaggattatcctgatgaggtgatcaacttcatgcgcagccacccactcatgtaccaggccgtgtaccctctgcagcggcggcccctggtagtccgcacaggtgctccctaccgccttaccactattgccgtggaccaggtggatgcagccgacgggcgctatgaggtgcttttcctgggcacagaccgcgggacagtgcagaaggtcattgtgctgcccaaggatgaccaggagatggaggagctcatgctggaggaggtggaggtcttcaaggatccagcacccgtcaagaccatgaccatctcttctaagaggcaacaactctacgtggcgtcagccgtgggtgtcacacacctgagcctgcaccgctgccaggcgtatggggctgcctgtgctgactgctgccttgcccgggacccttactgtgcctgggatggccaggcctgctcccgctatacagcatcctccaagaggcggagccgccggcaggacgtccggcacggaaaccccatcaggcagtgccgtgggttcaactccaatgccaacaagaatgccgtggagtctgtgcagtatggcgtggccggcagcgcagccttccttgagtgccagccccgctcgccccaagccactgttaagtggctgttccagcgagatcctggtgaccggcgccgagagattcgtgcagaggaccgcttcctgcgcacagagcagggcttgttgctccgtgcactgcagctcagcgatcgtggcctctactcctgcacagccactgagaacaactttaagcacgtcgtcacacgagtgcagctgcatgtactgggccgggacgccgtccatgctgccctcttcccaccactgtccatgagcgccccgccacccccaggcgcaggccccccaacgcctccttaccaggagttagcccagctgctggcccagccagaagtgggcctcatccaccagtactgccagggttactggcgccatgtgccccccagccccagggaggctccaggggcaccccggtctcctgagccccaggaccagaaaaagccccggaaccgccggcaccaccctccggacaca

SEQ ID NO: 5 - protein, 753 aa, Homo sapiens [sequence omitted]

SEQ ID NO: 6 - protein, 785 aa, Homo sapiens
MLVAGLLLWASLLTGAWPSFPTQDHLPATPRVRLSFKELKATGTAHFFNFLLNTTDYRILLKDEDHDRMYVGSKDYVLSLDLHDINREPLIIHWAASPQRIEECVLSGKDVNGECGNFVRLIQPWNRTHLYVCGTGAYNPMCTYVNRGRRAQATPWTQTQAVRGRGSRATDGALRPMPTAPRQDYIFYLEPERLESGKGKCPYDPKLDTASALINEELYAGVYIDFMGTDAAIFRTLGKQTAMRTDQYNSRWLNDPSFIHAELIPDSAERNDDKLYFFFRERSAEAPQSPAVYARIGRICLNDDGGHCCLVNKWSTFLKARLVCSVPGEDGIETHFDELQDVFVQQTQDVRNPVIYAVFTSSGSVFRGSAVCVYSMADIRMVFNGPFAHKEGPNYQWMPFSGKMPYPRPGTCPGGTFTPSMKSTKDYPDEVINFMRSHPLMYQAVYPLQRRPLVVRTGAPYRLTTIAVDQVDAGDGRYEVLFLGTDRGTVQKVIVLPKDDQEMEELMLEEVEVFKDPAPVKTMTISSKRQQLYVASAVGVTHLSLHRCQAYGAACADCCLARDPYCAWDGQACSRYTASSKRRSRRQDVRHGNPIRQCRGFNSNANKNAVESVQYGVAGSAAFLECQPRSPQATVKWLFQRDPGDRRREIRAEDRFLRTEQGLLLRALQLSDRGLYSCTATENNFKHVVTRVQLHVLGRDAVHAALFPPLSMSAPPPPGAGPPTPPYQELAQLLAQPEVGLIHQYCQGYWRHVPPSPREAPGAPRSPEPQDQKKPRNRRHHPPDT

SEQ ID NO: 7 - protein, 785 aa, Homo sapiens
MLVAGLLLWASLLTGAWPSFPTQDHLPATPRVRLSFKELKATGTAHFFNFLLNTTDYRILLKDEDHDRMYVGSKDYVLSLDLHDINREPLIIHWAASPQRIEECVLSGKDVNGECGNFVRLIQPWNRTHLYVCGTGAYNPMCTYVNRGRRAQATPWTQTQAVRGRGSRATDGALRPMPTAPRQDYIFYLEPERLESGKGKCPYDPKLDTASALINEELYAGVYIDFMGTDAAIFRTLGKQTAMRTDQYNSRWLNDPSFIHAELIPDSAERNDDKLYFFFRERSAEAPQSPAVYARIGRICLNDDGGHCCLVNKWSTFLKARLVCSVPGEDGIETHFDELQDVFVQQTQDVRNPVIYAVFTSSGSVFRGSAVCVYSMADIRMVFNGPFAHKEGPNYQWMPFSGKMPYPRPGTCPGGTFTPSMKSTKDYPDEVINFMRSHPLMYQAVYPLQRRPLVVRTGAPYRLTTIAVDQVDAADGRYEVLFLGTDRGTVQKVIVLPKDDQELEELMLEEVEVFKDPAPVKTMTISSKRQQLYVASAVGVTHLSLHRCQAYGAACADCCLARDPYCAWDGQACSRYTASSKRRSRRQDVRHGNPIRQCRGFNSNANKNAVESVQYGVAGSAAFLECQPRSPQATVKWLFQRDPGDRRREIRAEDRFLRTEQGLLLRALQLSDRGLYSCTATENNFKHVVTRVQLHVLGRDAVHAALFPPLSMSAPPPPGAGPPTPPYQELAQLLAQPEVGLIHQYCQGYWRHVPPSPREAPGAPRSPEPQDQKKPRNRRHHPPDT

SEQ ID NO: 8 - protein, 785 aa, Homo sapiens
MLVAGLLLWASLLTGAWPSFPTQDHLPATPRVRLSFKELKATGTAHFFNFLLNTTDYRILLKDEDHDRMYVGSKDYVLSLDLHDINREPLIIHWAASPQRIEECVLSGKDVNGECGNFVRLIQPWNRTHLYVCGTGAYNPMCTYVNRGRRAQATPWTQTQAVRGRGSRATDGALRPMPTAPRQDYIFYLEPERLESGKGKCPYDPKLDTASALINEELYAGVYIDFMGTDAAIFRTLGKQTAMRTDQYNSRWLNDPSFIHAELIPDSAERNDDKLYFFFRERSAEAPQSPAVYARIGRICLNDDGGHCCLVNKWSTFLKARLVCSVPGEDGIETHFDELQDVFVQQTQDVRNPVIYAVFTSSGSVFRGSAVCVYSMADIRMVFNGPFAHKEGPNYQWMPFSGKMPYPRPGTCPGGTFTPSMKSTKDYPDEVINFMRSHPLMYQAVYPLQRRPLVVRTGAPYRLTTIAVDQVDAADGRYEVLFLGTDRGTVQKVIVLPKDDQEMEELMLEEVEVFKDPAPVKTMTISSKRQQLYVASAVGVTHLSLHRCQAYGAACADCCLARDPYCAWDGQACSRYTASSKRRSRRQDVRHGNPIRQCRGFNSNANKNAVESVQYGVAGSAAFLECQPRSPQATVKWLFQRDPGDRRREIRAEDRFLRTEQGLLLRALQLSDRGLYSCTATENNFKHVVTRVQLHVLGRDAVHAALFPPLSMSAPPPPGAGPPTPPYQELAQLLAQPEVGLIHQYCQGYWRHVPPSPREAPGAPRSPEPQDQKKPRNRRHHPPDT

SEQ ID NO: 9 - DNA, 2259 bp, Homo sapiens; identical to SEQ ID NO: 1

SEQ ID NO: 10 - DNA, 2719 bp, Homo sapiens
cggggcccaggccccgccgctgcggaagaggtttctagagagtggagcctgcttcctgggccctaggcccctcccacaatgcttgtcgccggtcttcttctctgggcttccctactgaccggggcctggccatccttccccacccaggaccacctcccggccacgccccgggtccggctctcattcaaagagctgaaggccacaggcaccgcccacttcttcaacttcctgctcaacacaaccgactaccgaatcttgctcaaggacgaggaccacgaccgcatgtacgtgggcagcaaggactacgtgctgtccctggacctgcacgacatcaaccgcgagcccctcattatacactgggcagcctccccacagcgcatcgaggaatgcgtgctctcaggcaaggatgtcaacggcgagtgtgggaacttcgtcaggctcatccagccctggaaccgaacacacctgtatgtgtgcgggacaggtgcctacaaccccatgtgcacctatgtgaaccgcggacgccgcgcccaggccacaccatggacccagactcaggcggtcagaggccgcggcagcagagccacggatggtgccctccgcccgatgcccacagccccacgccaggattacatcttctacctggagcctgagcgactcgagtcagggaagggcaagtgtccgtacgatcccaagctggacacagcatcggccctcatcaatgaggagctctatgctggtgtgtacatcgattttatgggcactgatgcagccatcttccgcacacttggaaagcagacagccatgcgcacggatcagtacaactcccggtggctgaacgacccgtcgttcatccatgctgagctcattcctgacagtgcggagcgcaatgatgataagctttacttcttcttccgtgagcggtcggcagaggcgccgcagagccccgcggtgtacgcccgcatcgggcgcatttgcctgaacgatgacggtggtcactgttgcctggtcaacaagtggagcacattcctgaaggcgcggctcgtctgctctgtcccgggcgaggatggcattgagactcactttgatgagctccaggacgtgtttgtccagcagacccaggacgtgaggaaccctgtcatttacgctgtctttacctcctctggctccgtgttccgaggctctgccgtgtgtgtctactccatggctgatattcgcatggtcttcaacgggccctttgcccacaaagaggggcccaactaccagtggatgcccttctcagggaagatgccctacccacggccgggcacgtgccctggtggaaccttcacgccatctatgaagtccaccaaggattatcctgatgaggtgatcaacttcatgcgcagccacccactcatgtaccaggccgtgtaccctctgcagcggcggcccctggtagtccgcacaggtgctccctaccgccttaccactattgccgtggaccaggtggatgcaggcgacgggcgctatgaggtgcttttcctgggcacagaccgcgggacagtgcagaaggtcattgtgctgcccaaggatgaccaggagatggaggagctcatgctggaggaggtggaggtcttcaaggatccagcacccgtcaagaccatgaccatctcttctaagaggcaacaactctacgtggcgtcagccgtgggtgtcacacacctgagcctgcaccgctgccaggcgtatggggctgcctgtgctgactgctgccttgcccgggacccttactgtgcctgggatggccaggcctgctcccgctatacagcatcctccaagaggcggagccgccggcaggacgtccggcacggaaaccccatcaggcagtgccgtgggttcaactccaatgccaacaagaatgccgtggagtctgtgcagtatggcgtggccggcagcgcagccttccttgagtgccagccccgctcgccccaagccactgttaagtggctgttccagcgagatcctggtgaccggcgccgagagattcgtgcagaggaccgcttcctgcgcacagagcagggcttgttgctccgtgcactgcagctcagcgatcgtggcctctactcctgcacagccactgagaacaactttaagcacgtcgtcacacgagtgcagctgcatgtactgggccgggacgccgtccatgctgccctcttcccaccactgtccatgagcgccccgccacccccaggcgcaggccccccaacgcctccttaccaggagttagcccagctgctggcccagccagaagtgggcctcatccaccagtactgccagggttactggcgccatgtgccccccagccccagggaggctccaggggcaccccggtctcctgagccccaggaccagaaaaagccccggaaccgccggcaccaccctccggacacatgaggccagctgcctgtgcctgccatgggccaggctaggccttggtcccttttaatataaaagatatatatatatatatatatatatattaaaatatcggggtggggggtgattggaagggagggaggtggccttcccaatgcgcgttattcggggttattgaagaataatattgcaagtgacagccagaagtagactttctgtcctcacaccgaagaacccgagtgagcaggagggagggagagacgcgaagagaccttttttcctttttggagaccttgtccgc

SEQ ID NO: 11 - DNA, 2355 bp, Homo sapiens; identical to SEQ ID NO: 3

SEQ ID NO: 12 - DNA, 3054 bp, Homo sapiens
ccgcggcgccgatcccggctgaggcgcagcggcgagaggtcgcgggcagggccatggccccggggggccgctagcgcggaccggcccaacgggagccgctccgtgccgccgccgccgcccgggcgcccaggccccgccgctgcggaagaggtttctagagagtggagcctgcttcctgggccctaggcccctcccacaatgcttgtcgccggtcttcttctctgggcttccctactgaccggggcctggccatccttccccacccaggaccacctcccggccacgccccgggtccggctctcattcaaagagctgaaggccacaggcaccgcccacttcttcaacttcctgctcaacacaaccgactaccgaatcttgctcaaggacgaggaccacgaccgcatgtacgtgggcagcaaggactacgtgctgtccctggacctgcacgacatcaaccgcgagcccctcattatacactgggcagcctccccacagcgcatcgaggaatgcgtgctctcaggcaaggatgtcaacggcgagtgtgggaacttcgtcaggctcatccagccctggaaccgaacacacctgtatgtgtgcgggacaggtgcctacaaccccatgtgcacctatgtgaaccgcggacgccgcgcccaggccacaccatggacccagactcaggcggtcagaggccgcggcagcagagccacggatggtgccctccgcccgatgcccacagccccacgccaggattacatcttctacctggagcctgagcgactcgagtcagggaagggcaagtgtccgtacgatcccaagctggacacagcatcggccctcatcaatgaggagctctatgctggtgtgtacatcgattttatgggcactgatgcagccatcttccgcacacttggaaagcagacagccatgcgcacggatcagtacaactcccggtggctgaacgacccgtcgttcatccatgctgagctcattcctgacagtgcggagcgcaatgatgataagctttacttcttcttccgtgagcggtcggcagaggcgccgcagagccccgcggtgtacgcccgcatcgggcgcatttgcctgaacgatgacggtggtcactgttgcctggtcaacaagtggagcacattcctgaaggcgcggctcgtctgctctgtcccgggcgaggatggcattgagactcactttgatgagctccaggacgtgtttgtccagcagacccaggacgtgaggaaccctgtcatttacgctgtctttacctcctctggctccgtgttccgaggctctgccgtgtgtgtctactccatggctgatattcgcatggtcttcaacgggccctttgcccacaaagaggggcccaactaccagtggatgcccttctcagggaagatgccctacccacggccgggcacgtgccctggtggaaccttcacgccatctatgaagtccaccaaggattatcctgatgaggtgatcaacttcatgcgcagccacccactcatgtaccaggccgtgtaccctctgcagcggcggcccctggtagtccgcacaggtgctccctaccgccttaccactattgccgtggaccaggtggatgcagccgacgggcgctatgaggtgcttttcctgggcacagaccgcgggacagtgcagaaggtcattgtgctgcccaaggatgaccaggagatggaggagctcatgctggaggaggtggaggtcttcaaggatccagcacccgtcaagaccatgaccatctcttctaagaggcaacaactctacgtggcgtcagccgtgggtgtcacacacctgagcctgcaccgctgccaggcgtatggggctgcctgtgctgactgctgccttgcccgggacccttactgtgcctgggatggccaggcctgctcccgctatacagcatcctccaagaggcggagccgccggcaggacgtccggcacggaaaccccatcaggcagtgccgtgggttcaactccaatgccaacaagaatgccgtggagtctgtgcagtatggcgtggccggcagcgcagccttccttgagtgccagccccgctcgccccaagccactgttaagtggctgttccagcgagatcctggtgaccggcgccgagagattcgtgcagaggaccgcttcctgcgcacagagcagggcttgttgctccgtgcactgcagctcagcgatcgtggcctctactcctgcacagccactgagaacaactttaagcacgtcgtcacacgagtgcagctgcatgtactgggccgggacgccgtccatgctgccctcttcccaccactgtccatgagcgccccgccacccccaggcgcaggccccccaacgcctccttaccaggagttagcccagctgctggcccagccagaagtgggcctcatccaccagtactgccagggttactggcgccatgtgccccccagccccagggaggctccaggggcaccccggtctcctgagccccaggaccagaaaaagccccggaaccgccggcaccaccctccggacacatgaggccagctgcctgtgcctgccatgggccagcctagcccttgtcccttttaatataaaagatatatatatatatatatatatatatataaaatatctatattctatacacaccctgcccctgcaaagacagtatttattggtgggttgaatatagcctgcctcagtggcagcatcctccaaaacttagacccatgctggtcagagacggcagaaaacagagcctgcctaaccaggcccagccagttggtggggccaggccaggaccacacagtccccagactcagctggaagtctacctgctggacagcctccgccaagatctacaggacaaagggagggagcaagccctactcggatggggcacggactgtccaccttttctgatgtgtgttgtcagcctgtgctgtggcatagacatggatgcgaggaccactttggagactggggtggcctcaagagcacacagagaagggaagaaggggccatcacaggatgcca

SEQ ID NO: 13 - protein, 6 aa, Artificial Sequence
  Description of Artificial Sequence Synthetic 6xHis tag
HHHHHH

SEQ ID NO: 14 - protein, 8 aa, Artificial Sequence
  Description of Artificial Sequence Synthetic 8xHis tag
HHHHHHHH

(19) United States
(12) Patent Application Publication
(10) Pub. No.: US 2007/0054852 A1
(43) Pub. Date: Mar. 8, 2007

(54) Compositions and methods of use of Semaphorin 3F and antagonists thereof in the stimulation of neuromuscular regeneration and treatment of muscular diseases

(76) Inventors: Junyu Lin, Palo Alto, CA (US); Ge Wu, La Canada, CA (US); Srinivas Kothakota, Pacifica, CA (US); Lorianne Masuoka, Oakland, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/442,294

(22) Filed: May 30, 2006

Related U.S. Application Data
(60) Provisional application No. 60/685,702, filed on May 27, 2005.

(51) Int. Cl. A61K 38/17; C12Q 1/68

(52) U.S. Cl. 514/12; 435/6

(57) ABSTRACT

The present invention relates to therapeutic uses of Semaphorin 3F or related proteins. The therapeutic uses include methods of using Semaphorin 3F compounds alone, or in conjunction with other agents, for the stimulation of neuromuscular regeneration, and the treatment of diseases or conditions characterized by muscle denervation and muscle atrophy.

### CROSS REFERENCE TO RELATED APPLICATIONS

This application claims the benefit of U.S. Provisional Application 60/685,702, filed May 27, 2005; and the application filed under the Patent Cooperation Treaty May 30, 2006 titled “Methods of and Compositions for Stimulation of Glucose Uptake into Muscle Cells and Treatment of Disease,” the disclosures of which are incorporated in their entireties.

### FIELD OF THE INVENTION

The present invention relates to therapeutic uses of Semaphorin 3F and antagonists thereof. The therapeutic uses include methods of using compounds comprising Semaphorin 3F or antagonists thereof, for the modulation of interactions between muscle and nerve cells, the stimulation of neuromuscular regeneration, and the treatment of diseases or conditions characterized by muscle denervation or muscle atrophy.

### BACKGROUND OF THE INVENTION

Semaphorins represent a family of secreted or transmembrane glycoproteins that regulate cell motility and attachment in diverse processes such as axon guidance, vascular growth, immune cell regulation, and tumor progression (Kruger et al., Nat. Rev. Mol. Cell Biol. 6:789 (2005)). Semaphorins are grouped into eight classes based on differences in their structural elements and amino-acid sequence similarity. Classes 1 and 2 are the semaphorins found in invertebrates, classes 3 to 7 are the vertebrate semaphorins, and class 8 are semaphorins encoded by viruses. Semaphorins in classes 1, 4, 5, 6, and 7 are membrane-associated proteins, while class 2 and 3 semaphorins and the viral semaphorins are secreted proteins. Semaphorin 3F, the subject of the present invention, is a class 3 semaphorin and hence a secreted protein.

The main receptors for semaphorins are plexins. Plexins are transmembrane receptors which can function as ligand-binding receptors and as signalling receptors for semaphorins. Generally, interactions between plexins and semaphorins are mediated through the Sema domains of both proteins. One exception, however, is found in the case of class 3 semaphorins. Almost all class 3 semaphorins require neuropilins as semaphorin-binding co-receptors to signal through plexins. Neuropilins are transmembrane proteins which function as the ligand-binding partner in co-receptor complexes with both plexins and vascular endothelial growth factor receptors (VEGFRs). Once activated by semaphorins, plexins signal by regulating Rho-family GTPases and other signaling molecules.

Semaphorins regulate cell signaling, cell adhesion, and cell motility in different tissues. In the vascular system, both plexins and many class 3 semaphorins are expressed by endothelial cells. Hence, it has been proposed that class 3 semaphorin-mediated autocrine signalling reduces endothelial cell adhesion to stimulate endothelial cell motility that is required for vascular remodelling (Serini et al., Nature 424:391 (2003)). During the process of angiogenesis, the primary vascular plexus is remodelled into a mature vascular network. This vascular maturation requires that vessels fuse with each other to create larger vessels, and that vessels divide to form smaller vessels in a process called intussusception. It is thought that class 3 semaphorin-mediated autocrine stimulation of endothelial cells reduces their attachment via integrins, thereby enabling them to move effectively to either bridge or create gaps between adjacent vessels (Kruger et al., Nat. Rev. Mol. Cell Biol. 6:789 (2005)). Class 3 semaphorins are also essential for the formation of the heart (Gu et al., Dev. Cell 5:45 (2003)). Similar to the vascular defects, the heart defects resulting from defective semaphorin signalling are a consequence of abnormal cell migration. The regulation of integrins by plexin-activated R-Ras signaling is reported to underlie, at least in part, the effects of semaphorins on cell adhesion and migration.

Class 3 semaphorins also play a role in human cancer (Neufeld et al., Front. Biosci. 10:751 (2005)). The genes encoding semaphorin 3B and semaphorin 3F are located at chromosome 3p21.3 in humans, a region which is homozygously deleted in many small-cell lung cancer cell lines. Loss-of-heterozygosity in this region is frequently observed in lung and other cancers, and both genes are therefore candidate tumor suppressors. Semaphorin 3F is thought to regulate the metastatic properties of tumor cells, for example in lung carcinoma cells (Bielenberg et al., J. Clin. Invest. 114:1260 (2004)). Semaphorin 3F has been reported to regulate metastasis by stimulating plexin signalling, which in turn activates R-Ras, leading to reduced integrin binding. The precise role of semaphorins in cancer is still controversial, and there is evidence for both negative and positive regulation of metastasis by class 3 semaphorins (Kruger et al., Nat. Rev. Mol. Cell Biol. 6:789 (2005)). The different observations may relate to the fact that the effect of integrins on tumor growth and metastasis is very context-, type-, and stage-dependant.

The establishment of normal functional circuits in the mature nervous system requires the precise coordination of axon pathfinding and target recognition events during neural development. These recognition events are mediated by environmental cues that can either attract or repel axons and thereby regulate the formation of neuronal connections (Tessier-Lavigne and Goodman, Science 274:1123 (1996)). The axon attractants or repellents function, at least in part, by increasing or decreasing cell adhesion. Semaphorins are among the environmental cues that regulate neuronal development in such a fashion. In the developing nervous system, semaphorins have been reported to define areas from which plexin- and neuropilin-expressing neurons are excluded. Exceptions, however, have been noted. In addition, semaphorins are also reported to regulate axon branching.

In human patients, neuromuscular junctions are often irreversibly lost if the individuals are affected by a neuromuscular disease or by an injury, such as those resulting in paralysis. Damaged neuromuscular junctions may be regenerated in some situations, for example, if slow muscle fibers are involved. However, regeneration of neuromuscular junctions fails in many other instances, in particular, if fast-fatigable muscle fibers such as skeletal muscle fibers are involved. There is an urgent need in the art to find novel ways of treating neuromuscular diseases or neuromuscular defects due to injury. The instant invention relates to the novel use of semaphorin 3F in the direct regulation of muscle cells and the application of semaphorin 3F compounds in neuromuscular regeneration and the treament of neuromuscular diseases.

### SUMMARY OF THE INVENTION

Semaphorin genes and gene products regulate cell motility and attachment in diverse processes such as axon guidance, vascular growth, immune cell regulation and tumour progression. Semaphorins act to form and remodel cellular connections during these processes. Semaphorin 3F, a class 3 semaphorin, is a secreted protein previously reported to regulate certain cell types, which express a plexin receptor and a neuropilin co-receptor. The invention provides novel uses for semaphorin 3F in the regulation of muscle cells, including uses aimed at neuromuscular regeneration. The invention further provides nucleic acid molecules, polypeptides, expression vectors, host cells, fusion proteins, antibodies directed against, and transgenic animals that express semaphorin 3F, or a variant or fragment thereof. This includes a method of producing recombinant polypeptides of the invention by culturing host cells transformed with a recombinant expression vector under conditions appropriate for expression, then recovering the expressed polypeptide from the culture. The novel uses of semaphorin 3F include uses in diagnostic kits and therapeutics directed toward, for example, neuromuscular disorders and defects.

### BRIEF DESCRIPTION OF THE FIGURES AND TABLES

Brief Description of the Figures

FIG. 1 shows a flow chart of a high-throughput method used to screen unknown substances for significant effects on cell impedance, which is a measure of the cellular response to those substances.

FIG. 2 shows the results of screening human skeletal muscle cells with secreted factors for agents that increase impedance, as further described in Example 1. The graph shows the normalized cell index, expressed as the cell index, at a single time point (30 minutes). Columns 1-12 and rows A-H refer to the grid of wells in the 96 well plate. Betacellulin (arrow) is contained in well G3. Well H4 contains the internal positive control insulin growth factor-I (IGF-I). Well D10 contains Semaphorin 3F. Wells 12A-D contain the external positive control 10 nM IGF-1. No data are shown with respect to wells 1E-H and 2A-D.

FIG. 3 compares the amino acid sequences of Semaphorin 3F isoforms as provided by the National Center for Biotechnology Information (NCBI). The public semaphorin 3F sequences are aligned with the N— and C-terminal amino acid sequences of the semaphorin 3F polypeptide encoded by the cDNA clone identified in the impedance assay shown in FIG. 2. Sequences were aligned using clustal format for T-COFFEE Version_{—}1.37 with the parameters CPU=0.00 sec, SCORE=98, Nseq=4, Len=785. The NCBI accession numbers are provided to the left of the sequences. CLN00822499_{—}5pv1 and CLN00822499_{—}3pv1 represent the N— and C-terminal sequences of the identified semaphorin 3F polypeptide, respectively. Asterisks (*) indicate amino acid residues shared among all the sequences; colons (:) indicate conservative amino acid changes; and dashes (-) indicate absent amino acids.

FIG. 4 compares the cDNA sequences encoding Semaphorin 3F polypeptide isoforms (see FIG. 3) as provided by the National Center for Biotechnology Information (NCBI). The public semaphorin 3F nucleic acid sequences are aligned with the 5′ and 3′ portions of the Semaphorin 3F cDNA sequence identified in the impedance assay shown in FIG. 2. Sequences were aligned using clustal format for T-COFFEE Version_{—}1.37 with the parameters CPU=0.00 sec, SCORE=83, Nseq=6, Len=2355. The NCBI accession numbers are provided to the left of the sequences. CLN00822499_{—}5pv1 and CLN00822499_{—}3pv1 represent the 5′ and 3′ portions of the identified semaphorin 3F cDNA, respectively.

### BRIEF DESCRIPTION OF THE TABLES

Table 1 provides identification numbers for the Semaphorin 3F sequences listed in the Sequence Listing. Column 1 shows an internally designated identification number (FP ID). Column 2 shows the nucleotide sequence ID number for the nucleic acids of the open reading frames that encode the polypeptides of the invention (SEQ. ID. NO. (N1)). Column 3 shows the amino acid sequence ID number for polypeptide sequences (SEQ. ID. NO. (P1)). Column 4 shows the nucleotide sequence ID number for nucleic acids that may include both coding and non-coding regions (SEQ. ID. NO. (N0)). Column 5 shows the NCBI accession number for the nucleic acids and polypeptides specified in columns 2-4 (Clone ID).

Table 2 provides annotation and functional domain information for the Semaphorin 3F sequences. Column 1 shows the internally designated identification number (FP ID). Column 2 shows the NCBI accession number (Clone ID). Column 3 provides a list of predicted functional domains present in each of the identified sequences (Pfam Domains). Column 4 provides the coordinates of the Pfam domains (Pfam Coords). Column 5 shows the name and species origin of the sequence as listed in the NCBI database (Annotation).

The Pfam system is an organization of protein sequence classification and analysis, based on conserved protein domains. The Pfam system can be publicly accessed in a number of ways (for review and links to publicly available websites see Finn, R. D. et al. Nucleic Acids Res. 34:D247-D251, (2006)). Protein domains are portions of proteins that have a tertiary structure and sometimes have enzymatic or binding activities; multiple domains can be connected by flexible polypeptide regions within a protein. Pfam domains can comprise the N-terminus or the C-terminus of a protein, or can be situated at any point in between. The Pfam system identifies protein families based on these domains and provides an annotated, searchable database that classifies proteins into families.

Table 3 provides amino acid coordinates for the secreted Semaphorin 3F polypeptides. Column 1 shows the internally designated identification number (FP ID). Column 2 shows the predicted signal peptide coordinates (Signal Peptide Coords). Column 3 shows the mature protein coordinates, which refer to the coordinates of the amino acid residues of the mature polypeptide after cleavage of the secretory leader or signal peptide sequence (Mature Protein Coords). Column 4 provides the non-transmembrane coordinates (Non-TM Coords) which refer to those protein segments not located in the membrane; these can include extracellular, cytoplasmic, and luminal sequences. Column 5 shows alternate predictions of the signal peptide coordinates (Alternate Signal Peptide Coords). Column 6 specifies the coordinates of alternate forms of the mature protein (Alternate Mature Protein Coords). The alternate mature protein-coordinates result from alternative predictions of the signal peptide cleavage site; their presence may, for example, depend on the host used to express the polypeptides. All coordinates are listed in terms of the amino acid residues beginning with “1” for the first amino acid residue at the N-terminus of the full-length polypeptide.

### DETAILED DESCRIPTION OF THE INVENTION

### DEFINITIONS

The terms used herein have their ordinary meanings, as set forth below, and can be further understood in the context of the specification.

The terms “nucleic acid molecule,” “nucleotide,” “polynucleotide,” and “nucleic acid” are used interchangeably herein to refer to polymeric forms of nucleotides of any length. They can include both double- and single-stranded sequences and include, but are not limited to, cDNA from viral, prokaryotic, and eukaryotic sources; mRNA; genomic DNA sequences from viral (e.g. DNA viruses and retroviruses) or prokaryotic sources; RNAi; cRNA; antisense molecules; ribozymes; and synthetic DNA sequences. The term also captures sequences that include any of the known base analogs of DNA and RNA.

“Expression of a nucleic acid molecule” refers to the conversion of the information contained in the nucleic acid molecule into a gene product. The gene product can be the direct transcriptional product of a gene (e.g., mRNA, tRNA, rRNA, antisense RNA, ribozyme, structural RNA, or any other type of RNA) or a peptide or polypeptide produced by translation of an mRNA. Gene products also include RNAs which are modified by processes such as capping, polyadenylation, methylation, and editing; and proteins modified by, for example, methylation, acetylation, phosphorylation, ubiquitination, ADP-ribosylation, myristilation, and glycosylation.

A “vector” is an agent, typically a virus or a plasmid, which can be used to transfer genetic material to a cell or organism.

The terms “polypeptide” and “protein” are used interchangeably to refer to a polymer of amino acid residues, and are not limited to a minimum length. Thus, peptides, oligopeptides, dimers, multimers, and the like, are included within the definition. Both full-length proteins and fragments thereof are encompassed by the definition. The terms also include post-expression modifications of the polypeptide, for example, glycosylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

A “fragment” is any portion or subset of the corresponding polypeptide or polynucleotide molecule. Thus, for example, a “fragment of albumin” refers to a polypeptide subset of albumin and a “fragment of Fc” refers to a polypeptide subset of an Fc molecule. The term “fragment” is not intended to limit the portion or subset to any minimum or maximum length.

A “variant” of a first protein is meant to refer to a second protein that is substantially similar in structure and biological activity to either the native first protein or to a fragment thereof, but not identical to such molecule or fragment thereof. A variant is not necessarily derived from the native molecule and may be obtained from any of a variety of similar or different cell lines. The term “variant” is also intended to include genetic alleles and glycosylation variants. Thus, provided, for example, that two Semaphorin 3F proteins possess a similar structure and biological activity, they are considered variants as that term is used herein even if the composition or secondary, tertiary, or quaternary structure of one of the ligands is not identical to that found in the other.

The term “receptor” refers to a polypeptide that binds to a specific ligand. The ligand is usually an extracellular molecule which, upon binding to the receptor, usually initiates a cellular response, such as initiation of a signal transduction pathway. A “soluble receptor” is a receptor that lacks a membrane anchor domain, such as a transmembrane domain. A “soluble receptor” may include naturally occurring splice variants of a wild-type transmembrane protein receptor in which the transmembrane domain is spliced out. A “soluble receptor” may include the extracellular domain or any fragment of the extracellular domain of a transmembrane protein receptor. Soluble receptors can modulate a target protein. They can, for example, compete with wild-type receptors for ligand binding and participate in ligand/receptor interactions, thus modulating the activity of or the number of the receptors and/or the cellular activity downstream from the receptors. This modulation may trigger intracellular responses, for example, signal transduction events which activate cells, signal transduction events which inhibit cells, or events that modulate cellular growth, proliferation, differentiation, and/or death, or induce the production of other factors that, in turn, mediate such activities.

An “isolated,” “purified,” “substantially isolated,” or “substantially pure” molecule (such as a polypeptide or polynucleotide) is one that has been manipulated to exist in a higher concentration than in nature. For example, a subject antibody is isolated, purified, substantially isolated, or substantially purified when at least 10%, or 20%, or 40%, or 50%, or 70%, or 90% of non-subject-antibody materials with which it is associated in nature have been removed. As used herein, an “isolated,” “purified,” “substantially isolated,” or “substantially purified” molecule includes recombinant molecules.

A “biologically active” entity, or an entity having “biological activity,” is one having structural, regulatory, or biochemical functions of a naturally occurring molecule or any function related to or associated with a metabolic or physiological process. Biologically active polynucleotide fragments are those exhibiting activity similar, but not necessarily identical, to an activity of a polynucleotide of the present invention. The biological activity can include an improved desired activity, or a decreased undesirable activity. For example, an entity demonstrates biological activity when it participates in a molecular interaction with another molecule, such as hybridization, when it has therapeutic value in alleviating a disease condition, when it has prophylactic value in inducing an immune response, when it has diagnostic and/or prognostic value in determining the presence of a molecule, such as a biologically active fragment of a polynucleotide that can, for example, be detected as unique for the polynucleotide molecule, or that can be used as a primer in a polymerase chain reaction. A biologically active polypeptide or fragment thereof includes one that can participate in a biological reaction, including, but not limited to, one that can serve as an epitope or immunogen to stimulate an immune response, such as production of antibodies; or that can participate in modulating the immune response.

The terms “antibody” and “immunoglobulin” are used interchangeably to refer to a protein, for example, one generated by the immune system, synthetically, or recombinantly, that is capable of recognizing and binding to a specific antigen. Antibodies are commonly known in the art. Antibodies may recognize polypeptide or polynucleotide antigens. The term includes active fragments, including for example, an antigen binding fragment of an immunoglobulin, a variable and/or constant region of a heavy chain, a variable and/or constant region of a light chain, a complementarity determining region (cdr), and a framework region. The terms include polyclonal and monoclonal antibody preparations, as well as preparations including hybrid antibodies, altered antibodies, chimeric antibodies, hybrid antibody molecules, F(ab′)₂ and F(ab) fragments; Fv molecules (for example, noncovalent heterodimers), dimeric and trimeric antibody fragment constructs; minibodies, humanized antibody molecules, and any functional fragments obtained from such molecules, wherein such fragments retain specific binding.

The terms “binds specifically” or “specifically binds,” in the context of antibody binding, refers to high avidity and/or high affinity binding of an antibody to a specific epitope. Hence, an antibody that binds specifically to one epitope (a “first epitope”) and not to another (a “second epitope”) is a “specific antibody.” An antibody specific to a first epitope may cross react with and bind to a second epitope if the two epitopes share homology or other similarity. The term “binds specifically,” in the context of a polynucleotide, refers to hybridization under stringent conditions. Conditions that increase stringency of both DNA/DNA and DNA/RNA hybridization reactions are widely known and published in the art (Curr. Prot. Molec. Biol., John Wiley & Sons (2001)).

The term “agonist” refers to a substance that mimics or enhances the function of an active molecule. Agonists include, but are not limited to, antibodies, growth factors, cytokines, lymphokines, small molecule drugs, hormones, and neurotransmitters, as well as analogues and fragments thereof.

The term “antagonist” refers to a molecule that interferes with the activity or binding of another molecule such as an agonist, for example, by competing for the one or more binding sites of an agonist, but does not induce an active response.

The terms “subject,” “individual,” “host,” and “patient” are used interchangeably herein to refer to a living animal, including a human and a non-human animal. The subject may, for example, be an organism possessing immune cells capable of responding to antigenic stimulation, and stimulatory and inhibitory signal transduction through cell surface receptor binding. The subject may be a mammal, such as a human or non-human mammal, for example, dogs, cats, pigs, cows, sheep, goats, horses, rats, and mice. The term “subject” does not preclude individuals that are entirely normal with respect to a disease, or normal in all respects.

A “patient sample” is any biological specimen derived from a patient. The term includes, but is not limited to, biological fluids such as blood, serum, plasma, urine, cerebrospinal fluid, tears, saliva, lymph, dialysis fluid, lavage fluid, semen, and other liquid samples, as well as cell and tissues of biological origin. The term also includes cells or cells derived therefrom and the progeny thereof, including cells in culture, cell supernatants, and cell lysates. It further includes organ or tissue culture-derived fluids, tissue biopsy samples, tumor biopsy samples, stool samples, and fluids extracted from physiological tissues, as well as cells dissociated from solid tissues, tissue sections, and cell lysates. This definition encompasses samples that have been manipulated in any way after their procurement, such as by treatment with reagents, solubilization, or enrichment for certain components, such as polynucleotides or polypeptides. Also included in the term are derivatives and fractions of patient samples. A patient sample may be used in a diagnostic, prognostic, or other monitoring assay.

A “disease” is a pathological condition, for example, one that can be identified by symptoms or other identifying factors as diverging from a healthy or a normal state. The term “disease” includes disorders, syndromes, conditions, and injuries. Diseases include, but are not limited to, proliferative, inflammatory, immune, metabolic, infectious, and ischemic diseases.

The term “modulate” refers to the production, either directly or indirectly, of an increase or a decrease, a stimulation, inhibition, interference, or blockage in a measured activity when compared to a suitable control. A “modulator” of a polypeptide or polynucleotide or an “agent” are terms used interchangeably herein to refer to a substance that affects, for example, increases, decreases, stimulates, inhibits, interferes with, or blocks a measured activity of the polypeptide or polynucleotide, when compared to a suitable control.

“Prevention” or “Preventing,” as used herein, includes providing prophylaxis with respect to the occurrence or recurrence of a disease in a subject that may be predisposed to the disease but has not yet been diagnosed with the disease. Treatment and prophylaxis can be administered to an organism, including a human, or to a cell in vivo, in vitro, or ex vivo, and the cell subsequently administered the subject.

“Treatment” or “Treating,” as used herein, covers any administration or application of remedies for disease in a mammal, including a human, and includes inhibiting the disease. It includes arresting disease development and relieving the disease, such as by causing regression or restoring or repairing a lost, missing, or defective function, or stimulating an inefficient process.

A “carrier” refers to a solid, semisolid or liquid filler, diluent, encapsulating material, formulation auxiliary, or excipient of any conventional type. A “pharmaceutically acceptable carrier” refers to a non-toxic “carrier.” A pharmaceutically acceptable carrier is non-toxic to recipients at the dosages and concentrations employed and is compatible with other ingredients of the formulation. Pharmaceutically acceptable carriers can be, for example, vehicles, adjuvants, or diluents.

“Interfering RNA (RNAi)” refers to the effector molecules of RNA interference, a cellular mechanism of sequence-specific gene silencing that involves inhibition of gene transcription and/or translation. Interfering RNAs (RNAi) are short double-stranded RNA molecules that include, for example, small interfering RNAs (siRNAs) and microRNAs (miRNAs).

A “composition” or “pharmaceutical composition” herein refers to a composition that usually contains an excipient, such as a pharmaceutically acceptable carrier that is conventional in the art and that is suitable for administration into a subject for therapeutic, diagnostic, or prophylactic purposes. It can include a cell culture, in which the polypeptide or polynucleotide is present in the cells and/or in the culture medium. In addition, compositions for topical (e.g., oral mucosa, respiratory mucosa) and/or oral administration can form solutions, suspensions, tablets, pills, capsules, sustained-release formulations, oral rinses, or powders, as known in the art and described herein. The compositions also can include stabilizers and preservatives. For examples of carriers, stabilizers and adjuvants, University of the Sciences in Philadelphia (2005) Remington: The Science and Practice of Pharmacy with Facts and Comparisons, 21st ed.

As used herein, the term “kit” refers to components packaged or marked for use together. For example, a kit can contain two different agents and a carrier, and these three components can be in three separate containers. In another example, a kit can contain any two components in one container, and a third component and any additional components in one or more separate containers. Optionally, a kit further contains instructions for combining and/or administering the components so as to formulate a composition suitable for administration to a subject.

The terms “muscular disorders” or “muscular diseases” are intended to encompass muscular and neuromuscular disorders, some of which are characterized by a destabilization or improper organization of the plasma membrane of specific cell types and include muscular dystrophies (MDs). MDs are a group of genetic degenerative myopathies characterized by weakness and muscle atrophy without nervous system involvement.

It is to be understood that both the foregoing general description and the following detailed description are exemplary and explanatory only and are not restrictive of the invention, as claimed. Moreover, it must be understood that the invention is not limited to the particular embodiments described; such embodiments may, of course, vary. Further, the terminology used to describe particular embodiments is not intended to be limiting, since the scope of the present invention will be limited only by its claim.

### Identification of Semaphorin 3F as an Effector of Skeletal Muscle Cells

Using an impedance assay as described in detail in Example 1, several molecules were identified as novel, direct effectors of skeletal muscle cells, i.e., as compounds that directly interact with muscle cells and affect their cellular activity. One of these molecules is Semaphorin 3F. This result provided evidence for the expression of receptors for Semaphorin 3F in skeletal muscle cells and the direct regulation of skeletal muscle cells by Semaphorin 3F, which had hitherto been unknown. Since Semaphorin 3F is a molecule that regulates cell motility and cell interactions of its target cells, for example, in axon guidance during the formation of neural circuits and neuromuscular junctions, the present invention relates to Semaphorin 3F and related proteins, variants, fragments, and antagonists thereof, and methods of using these molecules to promote neuromuscular regeneration and to treat muscular disorders and defects due to injury, including neuromuscular disorders and defects. The invention accordingly provides compositions, and pharmaceutical combinations of compositions, comprising Semaphorin 3F or related proteins, variants, fragments, and antagonists thereof, and methods of using such compositions, for example, to stimulate neuromuscular regeneration or treat neuromuscular disorders and defects.

Semaphorin 3F and Related Nucleic acids and Polypeptides

The human Semaphorin 3F gene was isolated from chromosomal region 3p21.3, which displays homozygous deletions in small cell lung cancer cell lines (Xiang et al., Genomics 32:39 (1996)). Cancer-related mutations in the gene have not been reported to date. Expression of the gene is detected in various tissues including mammary gland, kidney, fetal brain, and lung.

Semaphorin polypeptides range in size from about 500 to about 1000 amino acids, depending on what domains they possess in addition to the Sema domain and the PSI (plexins, semaphorins and integrins) domain. The conserved Sema domain alone is between 400 and 500 amino acids in length. The structure of the Sema domain is similar to a domain found in integrins, and the Sema domain is also present in other molecules, such as plexins and the receptor tyrosine kinases Met and Ron. Semaphorin 3F, like other class 3 semaphorins, contains a single immunoglobulin-like domain (ig) carboxy-terminal to the Sema and PSI domains (see Table 2). Semaphorin 3F also contains multiple conserved cysteine residues (Kolodkin et al., Cell 75 75:1389 (1993)).

This invention provides Semaphorin 3F as a novel modulator of the interactions between muscle cells and nerve cells, mediated at least in part by direct effects on the muscle cells. The invention further provides methods of using Semaphorin 3F, or related factors, which include variants, mutants, and antagonists of Semaphorin 3F. Provided uses include the use as therapeutic agents for the treatment of muscular disorders, including neuromuscular disorders characterized by defective interactions between muscle and nerve cells. Therapeutic targets also include all other neuromuscular defects such as those caused by various insults, including injury or toxins.

Nucleic Acids

The present invention provides nucleic acid molecules comprising a polynucleotide sequence corresponding to one of the Semaphorin 3F sequences set forth in the Tables and Sequence Listing, for example, SEQ. ID. NOs.:1-4, or related polynucleotide sequences identified by methods described herein. The invention also provides uses for these nucleic acid molecules.

The invention provides recombinant DNA molecules that contain a promoter of a liver-expressed gene operably linked to a gene encoding Semaphorin 3F, or a related polypeptide, and that can be expressed in vivo to produce a protein that is functionally active. DNA molecules as described have a variety of uses, for example, as tools in basic research to study the in vivo function of an artificially introduced Semaphorin 3F, the interaction of more than one artificially introduced Semaphorin 3F, or the in vivo dynamics of artificially introduced Semaphorin 3F fusion proteins; as a tool to identify the in vivo targets of an artificially introduced Semaphorin 3F protein; or as therapeutic treatments, as further described below.

The present invention also provides nucleic acids that are related to the above DNA molecules and derived by processes such as transcription, splicing, processing, mutation, synthesis, chemical modification, or recombinant modification. Non-limiting embodiments or fragments of such nucleic acid molecules include genes or gene fragments, exons, introns, mRNA, tRNA, rRNA, siRNA, ribozymes, antisense nucleotide sequences, recombinant polynucleotides, branched polynucleotides, plasmids, vectors, isolated DNA of any sequence, isolated RNA of any sequence, nucleic acid probe sequences, and primer sequences. Such nucleic acid molecules or fragments thereof include splice variants of an mRNA; naturally occurring nucleotide sequences, for example, DNA or RNA; or synthetic analogs of purines and pyrimidines, as known in the art. Synthetic analogs may demonstrate increased stability under assay conditions. A nucleic acid molecule can also comprise modified nucleotides, such as methylated nucleotides or nucleotide analogs.

The present invention further relates to variants of the herein described nucleic acid molecules, which may occur naturally, such as a natural allelic variant, such as one of several alternate forms of a gene occupying a given locus on a chromosome of an organism, as described in, for example, Genes VIII, Lewin, B., ed., Prentice Hall (2003). Non-naturally occurring variants may be produced using mutagenesis techniques known in the art.

Such variants include those produced by nucleotide substitutions, deletions, or additions. The substitutions, deletions, or additions may involve one or more nucleotides. The variants may be altered in coding regions, non-coding regions, or both. Alterations in the non-coding regions may be such that the properties or activities of the gene regulatory elements, or portions thereof, are substantially the same. Alterations in the coding regions may produce conservative or non-conservative amino acid substitutions, deletions or additions. These may take the form of silent substitutions, additions, or deletions which do not alter the properties or activities of the encoded proteins, or portions thereof.

The present invention further relates to polynucleotides which hybridize to the hereinabove-described sequences if there is at least 91%, at least 92%, or at least 95% identity between the sequences. The present invention relates to polynucleotides which hybridize under stringent conditions to the hereinabove-described polynucleotides. Stringent conditions generally include condition under which hybridization will occur only if there is at least 95%, or at least 97% identity between the sequences. For example, overnight incubation at 42° C. in a solution containing 50% formamide, 5×SSC (150 mM NaCl, 15 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C., constitute stringent conditions.

Nucleic acids of the invention are useful as hybridization probes for differential identification of the tissue(s) or cell type(s) present in a biological sample. Fragments of the full length Semaphorin 3F sequence may be used as hybridization probes for cDNA libraries to isolate other genes which have a high sequence similarity or a substantially similar biological activity or function. Probes of this type can have at least 30 bases and may comprise, for example, 50 or more bases. The probe may also be used in a screening procedure to identify cDNA clones corresponding to full length transcripts and to genomic clones that contain complete Semaphorin 3F or related genes, including regulatory and promoter regions, exons, and introns. An example of such a screen would include isolating the coding regions of Semaphorin 3F or related genes by using a known nucleic acid sequence to synthesize an oligonucleotide probe. Labeled oligonucleotides having a sequence complementary to a gene of the present invention can be used to screen a human cDNA, a genomic DNA, or a mRNA library to identify complementary library components.

The polynucleotides which hybridize to the polynucleotides shown in the Tables and Sequence Listing can encode polypeptides which retain substantially similar biological function or activity as the provided Semaphorin 3F polypeptide. Alternatively, a polynucleotide may have at least 20 bases, at least 30 bases, or at least 50 bases which hybridize to a polynucleotide of the present invention and which has an identity thereto, and which may or may not retain substantially similar biological function or activity as the provided Semaphorin 3F polypeptide. Thus, the present invention is directed to polynucleotides having at least a 70% identity, at least an 80% identity, at least a 90% identity, or at least a 95% identity to a polynucleotide which encodes the Semaphorin 3F polypeptide set forth in the Appendix, as well as fragments thereof, which fragments have at least 30 bases or at least 50 bases, and to polypeptides encoded by such polynucleotides.

A polynucleotide having a nucleotide sequence at least, for example, 95% identical to a reference nucleotide sequence encoding a Semaphorin 3F polypeptide is one in which the nucleotide sequence is identical to the reference sequence except that it may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 70%, 80%, 90%, or 95% identical to, for instance, the nucleotide sequences set forth in the Appendix can be determined conventionally using known computer programs such as the Bestfit program (Wisconsin Sequence Analysis Package, Version 8 for Unix, Genetics Computer Group, Madison, Wis.). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

The present application is directed to nucleic acid molecules at least 70%, 80%, 90%, or 95% identical to the nucleic acid sequences set forth in the Appendix irrespective of whether they encode a polypeptide having Semaphorin 3F activity. Even where a particular nucleic acid molecule does not encode a polypeptide having Semaphorin 3F activity, one of skill in the art would know how to use the nucleic acid molecule, for instance, as a hybridization probe or a polymerase chain reaction (PCR) primer. Uses of the nucleic acid molecules of the present invention that do not encode a polypeptide having Semaphorin 3F activity include, inter alia, isolating the Semaphorin 3F gene or allelic variants thereof in a cDNA library; and in situ hybridization (for example, fluorescent in situ hybridization (FISH)) to metaphase chromosomal spreads to provide the precise chromosomal location of the Semaphorin 3F genes, as described in Verna et al., Human Chromosomes: A Manual of Basic Techniques, Pergamon Press, New York (1988); and Northern blot analysis for detecting Semaphorin 3F mRNA expression in specific tissues.

The present application is also directed to nucleic acid molecules having sequences at least 70%, 80%, 90%, or 95% identical to a nucleic acid sequence of the Appendix, which encode a polypeptide having Semaphorin 3F polypeptide activity, that is, a polypeptide exhibiting activity either identical to or similar to an activity of the Semaphorin 3F polypeptides of the invention, as measured in a particular biological assay. For example, the Semaphorin 3F polypeptides of the present invention may inhibit muscle cells in an impedance assay using real-time cell electronic sensing (RT-CES) technology (see FIG. 2).

Due to the degeneracy of the genetic code, one of ordinary skill in the art will immediately recognize that a large number of the nucleic acid molecules having a sequence at least 70%, 80%, 90%, or 95% identical to the nucleic acid sequence of the nucleic acid sequences set forth in the Appendix will encode a polypeptide having Semaphorin 3F polypeptide activity. In fact, since multiple degenerate variants of these nucleotide sequences encode the same polypeptide, this will be clear to the skilled artisan even without performing the above described comparison assay. It will be further recognized in the art that a reasonable number of nucleic acid molecules that are not degenerate variants will also encode a polypeptide having Semaphorin 3F polypeptide activity, the skilled artisan is fully aware of amino acid substitutions that are either less likely or not likely to significantly affect protein function (for example, replacing one aliphatic amino acid with a second aliphatic amino acid), as further described below.

Using the information provided herein, such as the nucleotide sequences set forth in the Tables and Appendix, nucleic acid molecules of the present invention encoding Semaphorin 3F or a related polypeptide may be obtained using standard cloning and screening procedures, such as those for cloning cDNAs using mRNA as starting material.

Vectors and Host Cells

The present invention also relates to vectors which include the nucleic acid sequences of the present invention, host cells which are genetically engineered with the recombinant vectors, and the production of Semaphorin 3F polypeptides or fragments thereof by recombinant techniques. It provides recombinant vectors that contain, for example, nucleic acid constructs that encode secretory leader sequences (for example, the secretory leader may be a collagen secretory leader), and a selected heterologous Semaphorin 3F related polypeptide of interest, and host cells that are genetically engineered with the recombinant vectors. The vector may be, for example, a phage, plasmid, or viral vector. Retroviral vectors may be replication competent or replication defective. In the latter case, viral propagation generally will occur only in complementing host cells. Vectors of the invention may contain Kozak sequences (Lodish et al., Molecular Cell Biology, 4^{th} ed., 1999). Vectors of the invention may also contain the ATG start codon of the sequence of interest.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The DNA insert can be operatively linked to an appropriate promoter, such as the phage lambda PL promoter; the E. coli lac, trp, phoA, and tac promoters; the SV40 early and late promoters; and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs can include a translation initiating codon at the beginning and a termination codon (UAA, UGA, or UAG) appropriately positioned at the end of the polypeptide to be translated.

The invention provides the expression of genes of interest in animals, including humans, under the control of a promoter that functions, inter alia, in the liver. The hydrodynamics-based procedure of tail vein injection (Zhang et al., Hum. Gene Ther. 10:1735 (1999)) has been demonstrated to transfect cells with a gene of interest. The invention also provides for the manipulation of the level of gene expression by controlling the amount and frequency of intravascular DNA administration. The invention further provides promoters that function to express genes in the liver.

One large family of proteins expressed in the liver is the cytochrome P450 protein family. These proteins are a group of heme-thiolate monooxygenases that perform a variety of oxidation reactions, often as part of the body's mechanism to dispose of harmful substances by making them more water-soluble. Much of the body's total mass of cytochrome P450 proteins is found in the liver, specifically, in the microsomes of hepatocytes. There are over a thousand different cytochrome P450 proteins. However, only 49 genes and 15 pseudogenes have been sequenced in humans. In humans, cytochrome P450 3A4 has been identified as the most important cytochrome P450 protein in oxidative metabolism. It is the most prevalent cytochrome P450 protein in the body, and is an inducible protein.

Operably linking the promoter sequence of genes expressed in the liver, for example, the promoter sequence of any of the cytochrome P450 proteins, to a gene of interest can lead to expression of that gene in the liver and any other site where the promoter is active. The invention encompasses promoters that function to express genes, including, but not limited to, cytochrome P450 gene, such as cytochrome P450 3A4; c-jun; jun-b; c-fos; c-myc; serum amyloid A; apolipoprotein B editing catalytic subunit; liver regeneration factors; such as LRF-1 signal transducers, and activators of transcription such as STAT-3; serum alkaline phosphatase (SAP); insulin-like growth factor-binding proteins such as IGFBP-1; cyclin D1; active protein-1; CCAAT enhancer core binding protein; beta ornithine decarbonylase; phosphatase of regenerating liver-1; early growth response gene-1; hepatocyte growth factors; hemopexin; insulin-like growth factors (IGF) such as IGF2; hepatocyte nuclear family 1; hepatocyte nuclear family 4; hepatocyte Arg-Ser-rich domain-containing proteins; glucose 6-phosphatase; and acute phase proteins, such as serum amyloid A and serum amyloid P (SAA/SAP).

Operably linking the promoter sequence of cytochrome P450 3A4 to Semaphorin 3F and injecting the construct into the tail vein of a mouse can be used to induce the expression of Semaphorin 3F. Thus, the invention provides therapeutic molecules of the invention, delivered in vivo. This method can be used to deliver naked DNA, in the presence or absence of a pharmaceutically acceptable carrier, or vector DNA with a sequence of interest. Methods of evaluating the function of the molecules of the invention delivered in vivo are known in the art, and some are described herein.

As indicated, the expression vectors may include at least one selectable marker. Such markers include dihydrofolate reductase, G418 or neomycin resistance for eukaryotic cell culture and tetracycline, kanamycin, or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

The selectable markers are genes that confer a phenotype on a cell expressing the marker, so that the cell can be identified under appropriate conditions. Generally, a selectable marker allows the selection of transformed cells based on their ability to thrive in the presence or absence of a chemical or other agent that inhibits an essential cell function. Suitable markers, therefore, include genes coding for proteins which confer drug resistance or sensitivity thereto, impart color to, or change the antigenic characteristics of those cells transfected with a molecule encoding the selectable marker, when the cells are grown in an appropriate selective medium. For example, selectable markers include cytotoxic markers and drug resistance markers, whereby cells are selected by their ability to grow on media containing one or more of the cytotoxins or drugs; auxotrophic markers by which cells are selected for their ability to grow on defined media with or without particular nutrients or supplements, such as thymidine and hypoxanthine; metabolic markers for which cells are selected, for example, their ability to grow on defined media containing the appropriate sugar as the sole carbon source, and markers which confer the ability of cells to form colored colonies on chromogenic substrates or cause cells to fluoresce.

Among vectors suitable for use in bacteria include pQE70, pQE60, and pQE-9, available from Qiagen, Mississauga, Ontario, Canada; pBS vectors, Phagescript vectors, Bluescript vectors, pNH8A, pNH6a, pNH18A, pNH46A, available from Stratagene (La Jolla, Calif.); and ptrc99a, pKK223-3, pKK233-3, pDR540, pRIT5 available from Pharmacia (Peapack, N.J.). Among suitable eukaryotic vectors are pWLNEO, pSV2CAT, pOG44, pXT1, and pSG available from Stratagene; and pSVK3, pBPV, pMSG and pSVL, available from Pharmacia. Other suitable vectors will be apparent to the skilled artisan.

Other suitable vectors include those employing a pTT vector backbone (Durocher et al. Nucl. Acids Res. 30 (2002)). Briefly, the pTT vector backbone may be prepared by obtaining pIRESpuro/EGFP (pEGFP) and pSEAP basic vector(s), for example from Clontech (Palo Alto, Calif.), and pcDNA3.1, pCDNA3.1/Myc-(His)₆ and pCEP4 vectors can be obtained from, for example, Invitrogen. As used herein, the pTT5 backbone vector can generate pTT5-Gateway and be used to transiently express proteins in mammalian cells. The pTT5 vector can be derivatized to pTT5-A, pTT5-B, pTT5-D, pTT5-E, pTT5-H, and pTT5-I, for example. As used herein, the pTT2 vector can generate constructs for stable expression in mammalian cell lines.

The expression vector pTT5 allows for extrachromosomal replication of the cDNA driven by a cytomegalovirus (CMV) promoter. The plasmid vector pCDNA-pDEST40 is a Gateway-adapted vector which can utilize a CMV promoter for high-level expression. SuperGlo GFP variant (sgGFP) can be obtained from Q-Biogene (Carlsbad, Calif.). Preparing a pCEP5 vector can be accomplished by removing the CMV promoter and polyadenylation signal of pCEP4 by sequential digestion and self-ligation using SalI and XbaI enzymes resulting in plasmid pCEP4Δ. A GblII fragment from pAdCMV5 (Massie et al., J. Virol., 72: 2289-2296 (1998)), encoding the CMV5-poly(A) expression cassette ligated in BglII-linearized pCEP4Δ, resulting in pCEP5 vector.

The pTT vector can be prepared by deleting the hygromycin (BsmI and SalI excision followed by fill-in and ligation) and EBNA1 (ClaI and NsiI excision followed by fill-in and ligation) expression cassettes. The ColEI origin (FspI-SalI fragment, including the 3′ end of β-lactamase ORF) can be replaced with a FspI-SalI fragment from pcDNA3.1 containing the pMBI origin (and the same 3′ end of β-lactamase ORF). A Myc-(His)₆ C-terminal fusion tag can be added to SEAP (HindIII-HpaI fragment from pSEAP-basic) following in-frame ligation in pcDNA3.1/Myc-His digested with HindIII and EcoRV. Plasmids can subsequently be amplified in E. coli (DH5α) grown in LB medium and purified using MAXI prep columns (Qiagen, Mississauga, Ontario, Canada). To quantify, plasmids can be subsequently diluted in 50 mM Tris-HCl pH 7.4 and absorbencies can be measured at 260 nm and 280 nm. Plasmid preparations with A₂₆₀/A₂₈₀ ratios between about 1.75 and about 2.00 are suitable.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Sambrook, J., et al. (2001) Molecular Cloning, A Laboratory Manual. 3^{nd} ed. Cold Spring Harbor Laboratory Press.

The polypeptides may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide.

Polypeptides

The invention further provides isolated Semaphorin 3F polypeptides containing the amino acid sequences encoded by the nucleotide sequences set forth in the Tables and Sequence Listing, for example, SEQ. ID. NOs.:5-8, which correspond to full-length polypeptides. The invention provides novel uses for these polypeptides and for related polypeptides described herein.

The invention provides secreted proteins, which are capable of being directed to the ER, secretory vesicles, or the extracellular space as a result of a secretory leader, signal peptide, or leader sequence, as well as proteins released into the extracellular space without necessarily containing a signal sequence. If a secreted protein is released into the extracellular space, it may undergo extracellular processing to a mature polypeptide. Release into the extracellular space can occur by many mechanisms, including exocytosis and proteolytic cleavage.

The Semaphorin 3F polypeptides can be recovered and isolated from recombinant cell cultures by well-known methods. Such methods include ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. High performance liquid chromatography (HPLC) can be employed for purification. Polypeptides of the present invention include products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in eukaryotic cells. While the N-terminal methionine on most proteins also is efficiently removed in most prokaryotes, for some proteins this prokaryotic removal process is inefficient, depending on the nature of the amino acid to which the N-terminal methionine is covalently linked.

Typically, a heterologous polypeptide, whether modified or unmodified, may be expressed as described above, or as a fusion protein, and may include a secretory leader sequence or other secretion signals. A secretory leader sequence of the invention directs certain proteins to the endoplasmic reticulum (ER). The ER separates the membrane-bound proteins from other proteins. Once localized to the ER, proteins can be further directed to the Golgi apparatus for distribution to vesicles, including secretory vesicles; the plasma membrane; lysosomes; and other organelles.

Proteins targeted to the ER by a secretory leader sequence can be released into the extracellular space as a secreted protein. For example, vesicles containing secreted proteins can fuse with the cell membrane and release their contents into the extracellular space via exocytosis. Exocytosis can occur constitutively or upon receipt of a triggering signal. In the latter case, the proteins may be stored in secretory vesicles (or secretory granules) until exocytosis is triggered. Similarly, proteins residing on the cell membrane can also be secreted into the extracellular space by proteolytic cleavage of a linker holding the protein to the membrane.

Additionally, peptide moieties and/or purification tags may be added to the polypeptide to facilitate purification. Such regions may be removed prior to final preparation of the polypeptide. The addition of peptide moieties to polypeptides to engender secretion or excretion, to improve stability, and to facilitate purification, among other reasons, are familiar and routine techniques in the art. Suitable purification tags include, for example, V5, HISX6, HISX8, avidin, and biotin.

The polypeptides of the present invention can be provided in an isolated form, and can be substantially purified, as described above. A recombinantly produced version of the herein described Semaphorin 3F polypeptides can also be substantially isolated, for example, according to the one-step method described in Smith and Johnson, Gene, 67:31-40 (1988). Polypeptides of the invention can further be isolated from natural or recombinant sources using anti-Semaphorin 3F antibodies of the invention produced using methods well known in the art.

The polypeptides herein may be purified or isolated in the presence of ions or agents that aid in the refolding of the molecules or aid in dimerizing or trimerizing the molecules as conventional in the art. For example, cofactors may be added to promote physiologic folding or multimerization.

Further polypeptides of the present invention include polypeptides which have at least 70%, 80%, 90%, or 95% identity to those described above. The polypeptides of the invention also contain those which are at least 70%, 80%, 90%, or 95% identical to a polypeptide encoded by a nucleic acid sequence of the Appendix.

The % identity of two polypeptides can be measured by a similarity score determined by comparing the amino acid sequences of the two polypeptides using the Bestfit program with the default settings for determining similarity. Bestfit uses the local homology algorithm of Smith and Waterman, Advances in Applied Mathematics 2:482-489 (1981) to find the best segment of similarity between two sequences.

A polypeptide having an amino acid sequence at least, for example, 95% identical to a reference amino acid sequence of a Semaphorin 3F polypeptide is one in which the amino acid sequence of the polypeptide is identical to the reference sequence except that the polypeptide sequence may include up to five amino acid alterations per each 100 amino acids of the reference polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids, up to 5% of the total amino acid residues in the reference sequence, may be inserted into the reference sequence. These alterations of the reference sequence may occur at the amino or carboxy terminal positions of the reference amino acid sequence or anywhere between those terminal positions, interspersed either individually among residues in the reference sequence, or in one or more contiguous groups within the reference sequence.

As a practical matter, whether any particular polypeptide is at least 70%, 80%, 90%, or 95% identical to, for instance, an amino acid sequence or to a polypeptide sequence encoded by a nucleic acid sequence set forth in the Appendix can be determined conventionally using known computer programs, such the Bestfit program. When using Bestfit or other sequence alignment program to determine whether a particular sequence is, for instance, 95% identical to a reference sequence according to the present invention, the parameters are set, of course, that the percentage of identity is calculated over the full length of the reference amino acid sequence and that gaps in homology of up to 5% of the total number of amino acid residues in the reference sequence are allowed.

Variant and Mutant Polypeptides

Protein engineering may be employed to improve or alter the characteristics of Semaphorin 3F polypeptides of the invention. Recombinant DNA technology known to those skilled in the art can be used to create novel mutant proteins or “muteins” including single or multiple amino acid substitutions, deletions, additions, or fusion proteins. Such modified polypeptides can show desirable properties, such as enhanced activity or increased stability. In addition, they may be purified in higher yields and show better solubility than the corresponding natural polypeptide, at least under certain purification and storage conditions.

N-Terminal and C-Terminal Deletion Mutants

For instance, for many proteins, including the extracellular domain of a membrane associated protein or the mature form(s) of a secreted protein, it is known in the art that one or more amino acids may be deleted from the N-terminus or C-terminus without substantial loss of biological function. For instance, Ron et al., J. Biol. Chem., 268:2984-2988 (1993), reported modified KGF proteins that had heparin binding activity even if 3, 8, or 27 amino-terminal amino acid residues were missing.

However, even if deletion of one or more amino acids from the N-terminus of a protein results in modification or loss of one or more biological functions of the protein, other biological activities may still be retained. Thus, the ability of the shortened protein to induce and/or bind to antibodies which recognize the complete or mature from of the protein generally will be retained when less than the majority of the residues of the complete or mature protein are removed from the N-terminus. Whether a particular polypeptide lacking N-terminal residues of a complete protein retains such immunologic activities can be determined by routine methods described herein and otherwise known in the art. Accordingly, the present invention may provide for polypeptides having one or more residues deleted from the amino terminus of the amino acid sequences of the Semaphorin 3F molecules as shown in the Appendix.

Similarly, many examples of biologically functional C-terminal deletion muteins are known. For instance, interferon gamma increases in activity as much as ten fold when 8-10 amino acid residues are deleted from the carboxy terminus of the protein, see, for example, Dobeli et al., J. Biotechnology, 7:199-216 (1988).

However, even if deletion of one or more amino acids from the C-terminus of a protein results in modification of loss of one or more biological functions of the protein, other biological activities may still be retained. Thus, the ability of the shortened protein to induce and/or bind to antibodies which recognize the complete or mature form of the protein generally will be retained when less than the majority of the residues of the complete or mature protein are removed from the C-terminus. Whether a particular polypeptide lacking C-terminal residues of a complete protein retains such immunologic activities can be determined by routine methods described herein and otherwise known in the art.

Cysteine to Serine Muteins

The Semaphorin 3F sequence includes several cysteine residues, located, for example, at amino acid positions 133, 142, 201, 300, 308, 309, 324, 372, 412, 548, 555, 558, 559, 566, 573, 598, 626, 678, and 746 of SEQ. ID. NO.:7. In an embodiment, the invention provides for mutant molecules with one or more than one of these cysteine residues mutated to serine. These mutant sequences may be cloned into any suitable vector, as known in the art, for example, the pTT5 vector.

Analyzing these muteins provides a better understanding of the possible intra- and inter-molecular disulfide bond pattern of Semaphorin 3F and may identify a protein with improved properties, for example, improved expression and secretion from mammalian cells, decreased aggregation of the purified protein, and the potential to produce active recombinant Semaphorin 3F, when expressed in E. coli.

Other Mutants

In addition to terminal deletion forms of the protein discussed above, it also will be recognized by one of ordinary skill in the art that some amino acid sequences of the Semaphorin 3F polypeptides can be varied without significant effect of the structure or function of the protein. If such differences in sequence are contemplated, it should be remembered that there will be critical areas on the protein which determine activity.

Thus, the invention further includes variations of the Semaphorin 3F polypeptides which show substantial Semaphorin 3F polypeptide activity or which include regions of the Semaphorin 3F proteins such as the protein portions discussed below. Such mutants include deletions, insertions, inversions, repeats, and type substitutions, selected according to general rules known in the art, so as have little effect on activity. For example, guidance concerning how to make phenotypically silent amino acid substitutions is provided in Bowie et al., Science, 247:1306-1310 (1990), wherein the authors indicate that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selections, or screens, to identify sequences that maintain functionality.

These studies report that proteins are surprisingly tolerant of amino acid substitutions. The authors further indicate which amino acid changes are likely to be permissive at a certain position of the protein. For example, most buried amino acid residues require nonpolar side chains, whereas few features of surface side chains are generally conserved. Other such phenotypically silent substitutions are described in Bowie, et al., supra, and the references cited therein. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg, and replacements between the aromatic residues Phe and Tyr.

Thus, a fragment, derivative, or analog of a polypeptide of the Appendix or polypeptide encoded by a nucleic acid sequence of the Appendix may be (i) one in which one or more of the amino acid residues are substituted with a conserved or non-conserved amino acid residue; such a substituted amino acid residue may or may not be one encoded by the genetic code; (ii) one in which one or more of the amino acid residues includes a substituent group; (iii) one in which the mature polypeptide is fused with another compound, such as a compound to increase the half-life of the polypeptide (for example, polyethylene glycol); or (iv) one in which the additional amino acids are fused to the above form of the polypeptide, such as an IgG Fc fusion region peptide, a leader or secretory sequence, a sequence employed to purify the above form of the polypeptide, or a proprotein sequence. Such fragments, derivatives, and analogs are deemed to be within the scope of those skilled in the art from the teachings herein.

Thus, the Semaphorin 3F polypeptides of the present invention may include one or more amino acid substitutions, deletions, or additions, either from natural mutations or human manipulation. As indicated, these changes may be of a minor nature, such as conservative amino acid substitutions, that do not significantly affect the folding or activity of the protein. Conservative amino acid substitutions include the aromatic substitutions Phe, Trp, and Tyr; the hydrophobic substitutions Leu, Iso, and Val; the polar substitutions Glu and Asp; the basic substitutions Arg, Lys, and His; the acidic substitutions Asp and Glu; and the small amino acid substations Ala, Ser, Thr, Met, and Gly.

Amino acids essential for the functions of Semaphorin 3F polypeptides can be identified by methods known in the art, such as site-directed mutagenesis or alanine-scanning mutagenesis, see, for example, Cunningham and Wells, Science, 244:1081-1085 (1989). The latter procedure introduces single alanine mutations. The resulting mutant molecules are then tested for biological activity such as modulation of muscle cell activity, for example, muscle cell impedance.

Of special interest are substitutions of charged amino acids with other charged or neutral amino acids which may produce proteins with highly desirable improved characteristics, such as less aggregation. Aggregation may not only reduce activity but also be problematic when preparing pharmaceutical formulations, because, for example, aggregates can be immunogenic, Pinckard et al., Clin. Exp. Immunol., 2:331-340 (1967); Robbins et al., Diabetes, 36:838-845 (1987); Cleland et al., Crit. Rev. Therapeutic Drug Carrier Systems, 10:307-377 (1993).

Replacing amino acids can also change the selectivity of the binding of a ligand to cell surface receptors. For example, Ostade et al., Nature, 361:266-268 (1993) describes mutations resulting in selective binding of TNF-α to only one of the two known types of TNF receptors. Sites that are critical for ligand-receptor binding can also be determined by structural analysis such as crystallization, nuclear magnetic resonance, or photoaffinity labeling, for example, Smith et al., J. Mol. Biol., 224:899-904 (1992) and de Vos et al., Science, 255:306-312 (1992).

Epitope-Bearing Portions

As described in detail below, the polypeptides of the present invention can be used to raise polyclonal and monoclonal antibodies, which are useful in assays for detecting Semaphorin 3F protein expression, also as described below, or as agonists and/or antagonists capable of enhancing or inhibiting Semaphorin 3F protein function. These polypeptides can also be used in a yeast two-hybrid system to capture Semaphorin 3F protein binding proteins, which are also candidate agonists and antagonists, according to the present invention. The yeast two hybrid system is described in Fields and Song, Nature, 340:245-246 (1989).

In another aspect, the invention provides a polypeptide comprising one or more epitope-bearing portions of a polypeptide of the invention. The invention provides polyclonal antibodies specific to Semaphorin 3F and provides that Semaphorin 3F has, at minimum, two antigenic epitopes. The epitope of this polypeptide portion is an immunogenic or antigenic epitope of a polypeptide of the invention. Immunogenic epitopes are those parts of a protein that elicit an antibody response when the whole protein is provided as the immunogen. On the other hand, a region of a protein molecule to which an antibody can bind is an antigenic epitope. The number of immunogenic epitopes of a protein generally is less than the number of antigenic epitopes. See, for instance, Geysen et al., Proc. Natl. Acad Sci., 81:3998-4002 (1983).

As to the selection of polypeptides bearing an antigenic epitope (that is, those which contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe et al., Science, 219:660-666 (1983). Peptides capable of eliciting protein-reactive sera are frequently represented in the primary sequence of a protein, can be characterized by a set of simple chemical rules, and are confined neither to immunodominant regions of intact proteins (that is, to immunogenic epitopes) nor to the amino or carboxyl terminals. Antigenic epitope-bearing peptides and polypeptides of the invention are therefore useful for raising antibodies, including monoclonal antibodies, that bind specifically to a polypeptide of the invention. See, for instance, Wilson et al., Cell, 37:767-778 (1984). The epitope-bearing peptides and polypeptides of the invention may be produced by any conventional means. See, for example, Houghten, Proc. Natl. Acad. Sci. 82:5131-5135 (1985), and U.S. Pat. No. 4,631,211 (1986).

Epitope-bearing peptides and polypeptides of the invention can be used to induce antibodies according to methods well known in the art. See, for instance, Bittle, et al, J. Gen. Virol., 66:2347-2354 (1985). Immunogenic epitope-bearing peptides of the invention, those parts of a protein that elicit an antibody response when the whole protein is the immunogen, are identified according to methods known in the art. See, for instance, U.S. Pat. No. 5,194,392 (1990), which describes a general method of detecting or determining the sequence of monomers (amino acids or other compounds) which is a topological equivalent of the epitope (mimotope) which is complementary to a particular antigen binding site (paratope) of an antibody of interest. More generally, U.S. Pat. No. 4,433,092 (1989) describes a method of detecting or determining a sequence of monomers which is a topographical equivalent of a ligand which is complementary to the ligand binding site of a particular receptor of interest. Similarly, U.S. Pat. No. 5,480,971 (1996) discloses linear C1-C7-alkyl peralkylated oligopeptides, and sets and libraries of such peptides, as well as methods for using such oligopeptide sets and libraries for determining the sequence of a peralkylated oligopeptide that, for example, binds to an acceptor molecule of interest. Thus, non-peptide analogs of the epitope-bearing peptides of the invention also can be made routinely by these methods.

Fusion Molecules

Gene manipulation techniques have enabled the development and use of recombinant therapeutic proteins with fusion partners that impart desirable pharmacokinetic properties. Several different fusion partners have been used to produce fusion molecules. The invention provides a fusion protein comprising a heterologous region that is useful to stabilize and/or purify proteins. Fusion molecules of the invention may comprise fusion partners that confer a longer in vivo half-life in a subject, compared to semaphorin 3A in the absence of a fusion partner. Suitable fusion partners include, but are not limited to, polymers, polypeptides, and succinyl groups. Polypeptide fusion partners of the invention include, but are not limited to, albumin, fragments of immunoglobulin molecules, and oligomerization domains. For example, recombinant human serum albumin fused with synthetic heme protein has been reported to reversibly carry oxygen (Chuang, V. T. et al., Pharm Res., 19:569-577 (2002)). The long half-life and stability of human serum albumin (HSA) makes it an attractive candidate for fusion to short-lived therapeutic proteins (U.S. Pat. No. 6,686,179).

The fusion of proteins with portions of an immunoglobulin molecule to improve stability and to facilitate purification, among others, are familiar and routine techniques in the art. For example, EP 0 464 533 (Canadian counterpart 2045869) discloses fusion proteins containing various portions of constant region of immunoglobulin molecules together with another human protein or part thereof. In many cases, the Fc part of a fusion protein is advantageous for use in therapy and diagnosis and thus results, for example, in improved pharmacokinetic properties (EP 0 232 262). On the other hand, for some uses it would be desirable to be able to delete the Fc part after the fusion protein has been expressed, detected, and purified in the advantageous manner described. This is the case when the Fc portion proves to be a hindrance to use in therapy and/or diagnosis, for example, when the fusion protein is to be used as an antigen for immunizations. In drug discovery, for example, human proteins, such as hIL-5, have been fused with Fc portions for the purpose of high-throughput screening assays to identify antagonists of hIL-5. See, Bennett et al., J. Molec. Recog., 8:52-58 (1995) and Johanson et al, J. Biol. Chem., 270:9459-9471 (1995).

As one of skill in the art will appreciate, Semaphorin 3F polypeptides of the present invention, and the epitope-bearing fragments thereof described above, can be combined with heterologous polypeptides, resulting in chimeric polypeptides. These fusion proteins facilitate purification and show an increased half-life in vivo. This has been reported, for example, in chimeric proteins consisting of the first two domains of the human CD4-polypeptide and various domains of the constant regions of the heavy or light chains of mammalian immunoglobulins (for example, EP 0 394 827; Traunecker et al., Nature, 331:84-86 (1988)). Fusion proteins that have a disulfide-linked dimeric structure due to the IgG portion can also be more efficient in binding and neutralizing other molecules than the monomeric protein or protein fragment alone, for example, as described by Fountoulakis et al., J. Biochem., 270:3958-3964 (1995). Suitable chemical moieties for derivatization of a heterologous polypeptide include, for example, polymers, such as water soluble polymers; the constant domain of immunoglobulins; all or part of human serum albumin; fetuin A; fetuin B; a leucine zipper domain; a tetranectin trimerization domain; mannose binding protein (also known as mannose binding lectin), for example, mannose binding protein 1; and an Fc region, as described herein and further described in U.S. Pat. No. 6,686,179, and U.S. Application Nos. 60/589,788 and 60/654,229. Methods of making fusion proteins are well-known to the skilled artisan.

For example, the short plasma half-life of unmodified interferon alpha makes frequent dosing necessary over an extended period of time, in order to treat viral and proliferative disorders. Interferon alpha fused with HSA has a longer half life and requires less frequent dosing than unmodified interferon alpha; the half-life was 18-fold longer and the clearance rate was approximately 140 times slower (Osborn et al., J. Pharmacol. Exp. Ther. 303:540-548, 2002). Interferon beta fused with HSA also has favorable pharmacokinetic properties; its half life was reported to be 36-40 hours, compared to eight hours for unmodified interferon beta (Sung et al., J. Interferon Cytokine Res. 23:25-36, 2003). An HSA-interleukin-2 fusion protein has been reported to have both a longer half-life and favorable biodistribution compared to unmodified interleukin-2. This fusion protein was observed to target tissues where lymphocytes reside to a greater extent than unmodified interleukin 2, suggesting that it exerts greater efficacy (Yao et al., Cancer Immunol. Immunother. 53:404-410, 2004).

The Fc receptor of human immunoglobulin G subclass 1 has also been used as a fusion partner for a therapeutic molecule. It has been recombinantly linked to two soluble p75 tumor necrosis factor (TNF) receptor molecules. This fusion protein has been reported to have a longer circulating half-life than monomeric soluble receptors, and to inhibit TNFα-induced proinflammatory activity in the joints of patients with rheumatoid arthritis (Goldenberg, Clin. Ther. 21:75-87, 1999). This fusion protein has been used clinically to treat rheumatoid arthritis, juvenile rheumatoid arthritis, psoriatic arthritis, and ankylosing spondylitis (Nanda and Bathon, Expert Opin. Pharmacother. 5:1175-1186, 2004).

Polymers, for example, water soluble polymers, are useful in the present invention as the polypeptide to which each polymer is attached will not precipitate in an aqueous environment, such as typically found in a physiological environment. Polymers employed in the invention will be pharmaceutically acceptable for the preparation of a therapeutic product or composition. One skilled in the art will be able to select the desired polymer based on such considerations as whether the polymer/protein conjugate will be used therapeutically and, if so, the desired dosage, circulation time, and resistance to proteolysis.

Suitable, clinically acceptable, water soluble polymers include, but are not limited to, polyethylene glycol (PEG), polyethylene glycol propionaldehyde, copolymers of ethylene glycol/propylene glycol, monomethoxy-polyethylene glycol, carboxymethylcellulose, dextran, polyvinyl alcohol (PVA), polyvinyl pyrrolidone, poly-1,3-dioxolane, poly-1,3,6-trioxane, ethylene/maleic anhydride copolymer, poly (β-amino acids) (either homopolymers or random copolymers), poly(n-vinyl pyrrolidone) polyethylene glycol, polypropylene glycol homopolymers (PPG) and other polyakylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll, or dextran and mixtures thereof.

As used herein, polyethylene glycol (PEG) is meant to encompass any of the forms that have been used to derivatize other proteins, such as mono-(C1-C10) alkoxy- or aryloxy-polyethylene glycol. Polyethylene glycol propionaldehyde may have advantages in manufacturing due to its stability in water.

Specifically, a modified heterologous polypeptide of the invention may be prepared by attaching polyaminoacids or branch point amino acids to the polypeptide. For example, the polyaminoacid may be a carrier protein that serves to increase the circulation half life of the polypeptide (in addition to the advantages achieved via a fusion molecule). For the therapeutic purpose of the present invention, such polyaminoacids should ideally be those that have or do not create neutralizing antigenic response, or other adverse responses. Such polyaminoacids may be chosen from serum album (such as human serum albumin); an additional antibody or portion thereof, for example the Fc region; fetuin A; fetuin B; leucine zipper nuclear factor erythroid derivative-2 (NFE2); neuroretinal leucine zipper; tetranectin; or other polyaminoacids, for example, lysines. As described herein, the location of attachment of the polyaminoacid may be at the N-terminus, or C-terminus, or other places in between, and also may be connected by a chemical linker moiety to the selected molecule.

Polymers used herein, for example water soluble polymers, may be of any molecular weight and may be branched or unbranched. The polymers each typically have an average molecular weight of between about 2 kDa to about 100 kDa (the term “about” indicating that in preparations of a polymer, some molecules will weigh more, some less, than the stated molecular weight). The average molecular weight of each polymer may be between about 5 kDa and about 50 kDa, or between about 12 kDa and about 25 kDa. Generally, the higher the molecular weight or the more branches, the higher the polymer:protein ratio. Other sizes may also be used, depending on the desired therapeutic profile; for example, the duration of sustained release; the effects, if any, on biological activity; the ease in handling; the degree or lack of antigenicity; and other known effects of a polymer on a modified molecule of the invention.

Polymers employed in the present invention are typically attached to a heterologous polypeptide with consideration of effects on functional or antigenic domains of the polypeptide. In general, chemical derivatization may be performed under any suitable condition used to react a protein with an activated polymer molecule. Activating groups which can be used to link the polymer to the active moieties include sulfone, maleimide, sulfhydryl, thiol, triflate, tresylate, azidirine, oxirane, and 5-pyridyl.

Polymers of the invention are typically attached to a heterologous polypeptide at the alpha (α) or epsilon (ε) amino groups of amino acids or a reactive thiol group, but it is also contemplated that a polymer group could be attached to any reactive group of the protein that is sufficiently reactive to become attached to a polymer group under suitable reaction conditions. Thus, a polymer may be covalently bound to a heterologous polypeptide via a reactive group, such as a free amino or carboxyl group. The amino acid residues having a free amino group may include lysine residues and the N-terminal amino acid residue. Those having a free carboxyl group may include aspartic acid residues, glutamic acid residues, and the C-terminal amino acid residue. Those having a reactive thiol group include cysteine residues.

Methods for preparing fusion molecules conjugated with polymers, such as water soluble polymers, will each generally involve (a) reacting a heterologous polypeptide with a polymer under conditions whereby the polypeptide becomes attached to one or more polymers and (b) obtaining the reaction product. Reaction conditions for each conjugation may be selected from any of those known in the art or those subsequently developed, but should be selected to avoid or limit exposure to reaction conditions such as temperatures, solvents, and pH levels that would inactivate the protein to be modified. In general, the optimal reaction conditions for the reactions will be determined case-by-case based on known parameters and the desired result. For example, the larger the ratio of polymer:polypeptide conjugate, the greater the percentage of conjugated product. The optimum ratio (in terms of efficiency of reaction in that there is no excess unreacted polypeptide or polymer) may be determined by factors such as the desired degree of derivatization (e.g., mono-, di-tri- etc.), the molecular weight of the polymer selected, whether the polymer is branched or unbranched and the reaction conditions used. The ratio of polymer (for example, PEG) to a polypeptide will generally range from 1:1 to 100:1. One or more purified conjugates may be prepared from each mixture by standard purification techniques, including among others, dialysis, salting-out, ultrafiltration, ion-exchange chromatography, gel filtration chromatography, and electrophoresis.

One may specifically desire an N-terminal chemically modified protein. One may select a polymer by molecular weight, branching, etc., the proportion of polymers to protein (polypeptide or peptide) molecules in the reaction mix, the type of reaction to be performed, and the method of obtaining the selected N-terminal chemically modified protein. The method of obtaining the N-terminal chemically modified protein preparation (separating this moiety from other monoderivatized moieties if necessary) may be by purification of the N-terminal chemically modified protein material from a population of chemically modified protein molecules.

Selective N-terminal chemical modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved. For example, one may selectively attach a polymer to the N-terminus of the protein by performing the reaction at a pH which allows one to take advantage of the pKa differences between the ε-amino group of the lysine residues and that of the α-amino group of the N-terminal residue of the protein. By such selective derivatization, attachment of a polymer to a protein is controlled: the conjugation with the polymer takes place predominantly at the N-terminus of the protein and no significant modification of other reactive groups, such as the lysine side chain amino groups, occurs. Using reductive alkylation, the polymer may be of the type described above and should have a single reactive aldehyde for coupling to the protein. Polyethylene glycol propionaldehyde, containing a single reactive aldehyde, may also be used.

In one embodiment, the present invention contemplates the chemically derivatized polypeptide to include mono- or poly- (e.g., 2-4) PEG moieties. Pegylation may be carried out by any of the pegylation reactions known in the art. Methods for preparing a pegylated protein product will generally include (a) reacting a polypeptide with polyethylene glycol (such as a reactive ester or aldehyde derivative of PEG) under conditions whereby the protein becomes attached to one or more PEG groups; and (b) obtaining the reaction product(s). In general, the optimal reaction conditions for the reactions will be determined case by case based on known parameters and the desired result.

There are a number of PEG attachment methods available to those skilled in the art. See, for example, EP 0 401 384; Malik et al., Exp. Hematol., 20:1028-1035 (1992); Francis, Focus on Growth Factors, 3(2):4-10 (1992); EP 0 154 316; EP 0 401 384; WO 92/16221; WO 95/34326; and the other publications cited herein that relate to pegylation.

The step of pegylation as described herein may be carried out via an acylation reaction or an alkylation reaction with a reactive polyethylene glycol molecule. Thus, protein products according to the present invention include pegylated proteins wherein the PEG group(s) is (are) attached via acyl or alkyl groups. Such products may be mono-pegylated or poly-pegylated (for example, those containing 2-6 or 2-5 PEG groups). The PEG groups are generally attached to the protein at the α- or ε-amino groups of amino acids, but it is also contemplated that the PEG groups could be attached to any amino group attached to the protein that is sufficiently reactive to become attached to a PEG group under suitable reaction conditions.

Pegylation by acylation generally involves reacting an active ester derivative of polyethylene glycol (PEG) with a polypeptide of the invention. For acylation reactions, the polymer(s) selected typically have a single reactive ester group. Any known or subsequently discovered reactive PEG molecule may be used to carry out the pegylation reaction. An example of a suitable activated PEG ester is PEG esterified to N-hydroxysuccinimide (NHS). As used herein, acylation is contemplated to include, without limitation, the following types of linkages between the therapeutic protein and a polymer such as PEG: amide, carbamate, urethane, and the like, see for example, Chamow, Bioconjugate Chem., 5:133-140 (1994). Reaction conditions may be selected from any of those known in the pegylation art or those subsequently developed, but should avoid conditions such as temperature, solvent, and pH that would inactivate the polypeptide to be modified.

Pegylation by acylation will generally result in a poly-pegylated protein. The connecting linkage may be an amide. The resulting product may be substantially only (e.g., >95%) mono, di- or tri-pegylated. However, some species with higher degrees of pegylation may be formed in amounts depending on the specific reaction conditions used. If desired, more purified pegylated species may be separated from the mixture (particularly unreacted species) by standard purification techniques, including among others, dialysis, salting-out, ultrafiltration, ion-exchange chromatography, gel filtration chromatography and electrophoresis.

Pegylation by alkylation generally involves reacting a terminal aldehyde derivative of PEG with a polypeptide in the presence of a reducing agent. For the reductive alkylation reaction, the polymer(s) selected should have a single reactive aldehyde group. An exemplary reactive PEG aldehyde is polyethylene glycol propionaldehyde, which is water stable, or mono C1-C10 alkoxy or aryloxy derivatives thereof, see for example, U.S. Pat. No. 5,252,714.

Additionally, heterologous polypeptides of the present invention and the epitope-bearing fragments thereof described herein can be combined with parts of the constant domain of immunoglobulins (IgG), resulting in chimeric polypeptides. These particular fusion molecules facilitate purification and show an increased half-life in vivo. This has been shown, for example, in chimeric proteins consisting of the first two domains of the human CD4-polypeptide and various domains of the constant regions of the heavy or light chains of mammalian immunoglobulins (EP 0 394 827; Traunecker et al., Nature, 331:84-86 (1988)). Fusion molecules that have a disulfide-linked dimeric structure due to the IgG part can also be more efficient in binding and neutralizing other molecules than, for example, a monomeric polypeptide or polypeptide fragment alone; see, for example, Fountoulakis et al., J. Biochem., 270:3958-3964 (1995).

Moreover, the polypeptides of the present invention can be fused to marker sequences, such as a peptide that facilitates purification of the fused polypeptide. The marker amino acid sequence may be a hexa-histidine peptide such as the tag provided in a pQE vector (Qiagen, Mississauga, Ontario, Canada), among others, many of which are commercially available. As described in Gentz et al., Proc. Natl. Acad. Sci. 86:821-824 (1989), for instance, hexa-histidine provides for convenient purification of the fusion protein. Another peptide tag useful for purification, the hemagglutinin HA tag, corresponds to an epitope derived from the influenza hemagglutinin protein. (Wilson et al., Cell 37:767 (1984)). Any of these above fusions can be engineered using the polynucleotides or the polypeptides of the present invention.

Secretory Leader Sequences

The invention provides Semaphorin 3F and related polypeptides, that are made to contain one, or more than one, heterologous secretory leader sequence, which may be derived from any secreted protein, including the ones listed herein. The provided heterologous secretory leader sequences facilitate the expression and secretion of Semaphorin 3F or other polypeptides of the invention.

As demonstrated, for example, in U.S. 60/647,013, in order for some secreted proteins to express and secrete in larger quantities, a secretory leader sequence from another, different, secreted protein is desirable. Employing heterologous secretory leader sequences is advantageous in that a resulting mature amino acid sequence of the secreted polypeptide is not altered as the secretory leader sequence is removed in the ER during the secretion process. Moreover, the addition of a heterologous secretory leader is required to express and secrete some proteins.

Identified secretory leader sequences are derived, for example, from collagen type IX alpha I chain, interleukin-9 precursor, T cell growth factor P40, P40 cytokine, triacylglycerol lipase, pancreatic precursor, somatoliberin precursor, vasopressin-neurophysin 2-copeptin precursor, beta-enoendorphin-dynorphin precursor, complement C2 precursor, small inducible cytokine A14 precursor, elastase 2A precursor, plasma serine protease inhibitor precursor, granulocyte-macrophage colony-stimulating factor precursor, interleukin-2 precursor, interleukin-3 precursor, alpha-fetoprotein precursor, alpha-2-HS-glycoprotein precursor, serum albumin precursor, inter-alpha-trypsin inhibitor light chain, serum amyloid P-component precursor, apolipoprotein A-II precursor, apolipoprotein D precursor, colipase precursor, carboxypeptidase A1 precursor, alpha-s1 casein precursor, beta casein precursor, cystatin SA precursor, follitropin beta chain precursor, glucagon precursor, complement factor H precursor, histidine-rich glycoprotein precursor, interleukin-5 precursor, alpha-lactalbumin precursor, Von Ebner's gland protein precursor, matrix Gla-protein precursor, alpha-1-acid glycoprotein 2 precursor, phospholipase A2 precursor, dendritic cell chemokine 1, statherin precursor, transthyretin precursor, apolipoprotein A-1 precursor, apolipoprotein C-III precursor, apolipoprotein E precursor, complement component C8 gamma chain precursor, serotransferrin precursor, beta-2-microglobulin precursor, neutrophils defensins 1 precursor, triacylglycerol lipase gastric precursor, haptoglobin precursor, neutrophils defensins 3 precursor, neuroblastoma suppressor of tumorigenicity 1 precursor, small inducible cytokine A13 precursor, CD5 antigen-like precursor, phospholipids transfer protein precursor, dickkopf related protein-4 precursor, elastase 2B precursor, alpha-1-acid glycoprotein 1 precursor, beta-2-glycoprotein 1 precursor, neutrophil gelatinase-associated lipocalin precursor, C-reactive protein precursor, interferon gamma precursor, kappa casein precursor, plasma retinol-binding protein precursor, and interleukin-13 precursor.

The secretory leader sequences listed herein are useful in the expression of a wide variety of polypeptides, including, for example, secreted polypeptides, extracellular proteins, transmembrane proteins, and receptors, such as soluble receptors. Descriptions of some proteins that can be expressed using such secretory leader sequences may be found in, for example, Human Cytokines: Handbook for Basic and Clinical Research, Vol. II (Aggarwal and Gutterman, eds. Blackwell Sciences, Cambridge Mass., 1998); Growth Factors: A Practical Approach (McKay and Leigh, eds., Oxford University Press Inc., New York, 1993) and The Cytokine Handbook (A. W. Thompson, ed.; Academic Press, San Diego Calif.; 1991).

Co-Translational and Post-Translational Modifications

The invention encompasses Semaphorin 3F polypeptides, or related polypeptides, which are differentially modified during or after translation, for example by glycosylation, acetylation, phosphorylation, amidation, derivatization by known protecting/blocking groups, proteolytic cleavage, or linkage to an antibody molecule or other cellular ligand. Any of numerous chemical modifications may be carried out by known techniques, including, but not limited to, specific chemical cleavage by cyanogen bromide, trypsin, chymotrypsin, papain, V8 protease; NABH₄; acetylation; formylation; oxidation; reduction; and/or metabolic synthesis in the presence of tunicamycin.

Additional post-translational modifications encompassed by the invention include, for example, for example, N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The polypeptides may also be modified with a detectable label, such as an enzymatic, fluorescent, isotopic, or affinity label to allow for detection and isolation of the protein.

Also provided by the invention are chemically modified derivatives of the polypeptides of the invention which may provide additional advantages such as increased solubility, stability, and circulating time of the polypeptide, or decreased immunogenicity (see U.S. Pat. No. 4,179,337). The chemical moieties for derivitization may be chosen from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three, or more attached chemical moieties.

Identification of Agonists and Antagonists

The invention provides modulators, including polypeptides, polynucleotides, and other agents that increase or decrease the activity of their target molecule. The target molecules of these modulators are Semaphorin 3F and/or any related molecule, including those polypetides and nucleic acids provided in the Tables and Sequence Listing. The modulators allow the manipulation of the biological activity of their targets in vitro or in vivo, for example, the regulation of muscle cells by Semaphorin 3F.

Modulators of the invention may act as an agonist or antagonist, and may interfere with the binding or activity of semaphorin 3A polypeptides or polynucleotides. Such modulators, or agents, include, for example, polypeptide variants, whether agonist or antagonist; antibodies, whether agonist or antagonist; soluble receptors, usually antagonists; small molecule drugs, whether agonist or antagonist; RNAi, usually an antagonist; antisense molecules, usually an antagonist; and ribozymes, usually an antagonist. In some embodiments, an agent is a polypeptide, which is administered to an individual. In some embodiments, an agent is an antibody specific for a target polypeptide. In some embodiments, an agent is a soluble receptor that specifically binds to a target polypeptide. In some embodiments, an agent is a chemical compound, such as a small molecule, that may be useful as an orally available drug. Such modulation includes the recruitment of other molecules that directly effect the modulation. An agent which modulates a biological activity of a target polypeptide or polynucleotide increases or decreases the activity or binding at least about 10%, at least about 15%, at least about 20%, at least about 25%, at least about 50%, at least about 80%, or at least about 2-fold, at least about 5-fold, or at least about 10-fold or more when compared to a suitable control.

The invention also provides a method of screening compounds to identify those which modulate the biological activity of a polypeptide or nucleic acid of the present invention. Examples of the biological activities of the polypeptides and nucleic acids of the invention are described in greater detail herein, for example in the Examples and the Figures.

Examples of antagonistic compounds include antibodies, or in some cases, oligonucleotides, which bind to the Semaphorin 3F polypeptide itself. Alternatively, a potential antagonist may be a mutant form of the Semaphorin 3F polypeptide which binds to an identical interacting molecule but without eliciting a biological response, thus effectively blocking the action of the native Semaphorin 3F.

Other potential antagonists include soluble receptors which bind to Semaphorin 3F. In some embodiments, the antagonist is an extracellular domain of a Semaphorin 3F receptor or co-receptor, selected from the group including plexins and neuropilins. In some embodiments, the antagonist is an extracellular domain of a Semaphorin 3F receptor or co-receptor which is fused to a second polypeptide to increase stability and in vivo half-life. The fusion moiety may be a Fc fragment, an albumin polypeptide, or any other useful fusion molecule selected from the fusion molecules described above.

Other potential antagonists include antisense molecules. Antisense technology can be used to control gene expression through, for example, antisense DNA or RNA, or through triple-helix formation. Antisense techniques are discussed, for example, in Okano, J. Neurochem., 56:560 (1991); Oligodeoxynucleotides as Antisense Inhibitors of Gene Expression, CRC Press, Boca Raton, Fla. (1988). Triple helix formation is discussed in, for instance, Lee et al., Nucleic Acids Research, 6:3073 (1979); Cooney et al., Science, 241:456 (1988); and Dervan et al., Science, 251:1360 (1991). The methods are based on the binding of a polynucleotide to a complementary DNA or RNA. For example, the 5′ coding portion of a polynucleotide that encodes the mature polypeptide of the present invention may be used to design an antisense RNA oligonucleotide of from about 10 to about 40 base pairs in length. A DNA oligonucleotide is designed to be complementary to a region of the gene involved in transcription, thereby preventing transcription and the subsequent production of Semaphorin 3F molecules. The antisense RNA oligonucleotide hybridizes to the mRNA in vivo and blocks translation of the mRNA molecule into a Semaphorin 3F polypeptide. The oligonucleotides described above can also be delivered to cells such that the antisense RNA or DNA may be expressed in vivo to inhibit production of Semaphorin 3F molecules.

Other potential antagonists include interfering RNAs (RNAi) against Semaphorin 3F or related molecules. RNA interference provides a method of silencing eukaryotic genes (Dykxhoorn et al., Nat. Rev. Mol. Cell Biol. 4:457 (2003)). Use of RNAi to reduce a level of a particular mRNA and/or protein is based on the interfering properties of RNA, for example, double-stranded RNA (dsRNA), derived from the coding regions of a gene. The technique is an efficient method for disrupting gene function. Accordingly, the invention provides as antagonistic modulators RNAi molecules that inhibit the transcription and/or translation of Semaphorin 3F or related molecules.

Potential antagonist compounds also include small molecules which bind to Semaphorin 3F in such a fashion that molecular interactions that are essential for its normal biological activity are blocked. Examples of small molecules include, but are not limited to, small peptides or peptide-like molecules and non-peptide chemical moieties. Antagonist compounds may be employed to inhibit the effects of the polypeptides of the invention, described in further detail in the Examples and Figures. The antagonists may be employed to diagnose, determine a prognosis for, prevent, and treat muscle-related diseases, as described in further detail below.

The present invention also provides methods for identifying agents, such as antibodies, which enhance or block the actions of Semaphorin 3F molecules on cells. For example, these agents may enhance or block interaction of Semaphorin 3F-binding molecules, such as receptors. Agents of interest include both agonists and antagonists. The invention provides agonists which increase the natural biological functions of Semaphorin 3F or which function in a manner similar to Semaphorin 3F. The invention also provides antagonists, which decrease or eliminate the functions of Semaphorin 3F.

One method of identifying Semaphorin 3F agonists and antagonists involves biochemical assays following subcellular fractionation. For example, a cellular compartment, such as a membrane or cytosolic preparation may be prepared from a cell that expresses a molecule that binds Semaphorin 3F molecules, such as a molecule of a regulatory pathway modulated by Semaphorin 3F molecules. Subcellular fractionation methods are known in the art of cell biology, and can be tailored to produce crude fractions with discrete and defined components, for example, organelles or organellar membranes. The preparation is incubated with labeled Semaphorin 3F molecules in the absence or the presence of a candidate molecule which may be an Semaphorin 3F agonist or antagonist. The ability of the candidate molecule to interact with the binding molecule or an Semaphorin 3F molecules is reflected in decreased binding of the labeled ligand. Molecules which bind gratuitously, that is, without inducing the effects of Semaphorin 3F molecules, are most likely antagonists. Molecules that bind well and elicit effects that are the same as or closely related to Semaphorin 3F molecules may potentially prove to be agonists.

The effects of potential agonists and antagonists may by measured, for instance, by comparing an activity of the target molecule in absence or presence of the modulator. This may include testing the effects of the potential modulator on the inhibition of muscle cell index by Semaphorin 3F in an impedance assay, as described in more detail in the Figures and Examples.

Therapeutic Compositions and Formulations

The nucleic acids, vectors, polypeptides, agonists, antagonists, and host cells of the present invention may be employed in combination with a suitable pharmaceutical carrier, or excipient, to comprise a pharmaceutical composition for parenteral administration. Such compositions comprise a therapeutically effective amount of the nucleic acid, vector, polypeptide, agonist, antagonist, or host cell and a pharmaceutically acceptable carrier or excipient. Such a carrier includes, but is not limited to, saline, buffered saline, dextrose, water, glycerol, ethanol, and combinations thereof. In addition, if desired, the vehicle can contain minor amounts of auxiliary substances such as wetting or emulsifying agents or pH buffering agents. Actual methods of preparing such dosage forms are known, or will be apparent, to those skilled in the art. The composition or formulation to be administered will contain a quantity of the agent adequate to achieve the desired state in the subject being treated. The formulation should suit the mode of administration.

In some embodiments, Semaphorin 3F compositions are provided in formulation with pharmaceutically acceptable excipients, a wide variety of which are known in the art (Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Pharmaceutically acceptable excipients, such as vehicles, adjuvants, carriers or diluents, are available to the public. Moreover, pharmaceutically acceptable auxiliary substances, such as pH adjusting and buffering agents, tonicity adjusting agents, stabilizers, wetting agents and the like, are available to the public.

The Semaphorin 3F polypeptide compositions will be formulated and dosed in a fashion consistent with good medical practice, taking into account the clinical condition of the individual subject, the site of delivery of the Semaphorin 3F polypeptide composition, the method of administration, the scheduling of administration, and other factors known to practitioners. The effective amount of Semaphorin 3F polypeptide for purposes herein is thus determined by such considerations.

In pharmaceutical dosage forms, the compositions of the invention can be administered in the form of their pharmaceutically acceptable salts, or they can also be used alone or in appropriate association, as well as in combination, with other pharmaceutically active compounds. The subject compositions are formulated in accordance to the mode of potential administration. Administration of the agents can be achieved in various ways, including oral, buccal, nasal, rectal, parenteral, intraperitoneal, intradermal, transdermal, subcutaneous, intravenous, intra-arterial, intracardiac, intraventricular, intracranial, intratracheal, and intrathecal administration, etc., or otherwise by implantation or inhalation. Thus, the subject compositions can be formulated into preparations in solid, semi-solid, liquid or gaseous forms, such as tablets, capsules, powders, granules, ointments, solutions, suppositories, enemas, injections, inhalants and aerosols. The following methods and excipients are merely exemplary and are in no way limiting.

The pharmaceutical compositions may be administered in a convenient manner such as by the oral, topical, intravenous, intraperitoneal, intramuscular, subcutaneous, intranasal, or intradermal routes. The pharmaceutical compositions are administered in an amount which is effective for treating and/or prophylaxis of the specific indication. In general, they are administered in an amount of at least about 10 micrograms/kg body weight and in most cases they will be administered in an amount not in excess of about 8 milligrams/kg body weight per day.

Compositions for oral administration can form solutions, suspensions, tablets, pills, granules, capsules, sustained release formulations, oral rinses, or powders. For oral preparations, the agents, polynucleotides, and polypeptides can be used alone or in combination with appropriate additives, for example, with conventional additives, such as lactose, mannitol, corn starch, or potato starch; with binders, such as crystalline cellulose, cellulose derivatives, acacia, corn starch, or gelatins; with disintegrators, such as corn starch, potato starch, or sodium carboxymethylcellulose; with lubricants, such as talc or magnesium stearate; and if desired, with diluents, buffering agents, moistening agents, preservatives, and flavoring agents.

The agents, polynucleotides, and polypeptides can be formulated into preparations for injection by dissolving, suspending, or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Other formulations for oral or parenteral delivery can also be used, as conventional in the art.

The antibodies, agents, polynucleotides, and polypeptides can be utilized in aerosol formulation to be administered via inhalation. The compounds of the present invention can be formulated into pressurized acceptable propellants such as dichlorodifluoromethane, propane, nitrogen, and the like. Further, the agent, polynucleotides, or polypeptide composition may be converted to powder form for administration intranasally or by inhalation, as conventional in the art.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions can be provided wherein each dosage unit, for example, teaspoonful, tablespoonful, tablet, or suppository, contains a predetermined amount of the composition containing one or more agents. Similarly, unit dosage forms for injection or intravenous administration can comprise the agent(s) in a composition as a solution in sterile water, normal saline, or another pharmaceutically acceptable carrier.

The polypeptides of the invention, and agonist and antagonist compounds which are polypeptides, may also be employed in accordance with the present invention by expression of such polypeptides in vivo, i.e., in a gene therapy approach. Thus, for example, cells may be engineered with a polynucleotide (DNA or RNA) encoding for the polypeptide ex vivo; the engineered cells are then provided to a patient. Such methods are well-known in the art. For example, cells may be engineered by procedures known in the art by use of a retroviral particle containing RNA encoding for the polypeptide of the present invention.

A polynucleotide, polypeptide, or other modulator, can also be introduced into tissues or host cells by other routes, such as viral infection, microinjection, or vesicle fusion. For example, expression vectors can be used to introduce nucleic acid compositions into a cell as described above. Further, jet injection can be used for intramuscular administration (Furth et al., Anal. Biochem. 205:365-368 (1992)). The DNA can be coated onto gold microparticles, and delivered intradermally by a particle bombardment device, or “gene gun” as described in the literature (Tang et al., Nature 356:152-154 (1992)), where gold microprojectiles are coated with the DNA, then bombarded into skin cells.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the polypeptides, agonists and antagonists of the present invention may be employed in conjunction with other therapeutic compounds.

Diagnosis

This invention is also related to the use of the genes and gene products of the present invention as part of a diagnostic assay for detecting diseases or susceptibility to diseases related to the presence of mutations in the nucleic acid sequences encoding Semaphorin 3F or related polypeptides of the present invention. Individuals carrying mutations in a gene of the present invention may be detected at the DNA level by a variety of techniques. Nucleic acids for diagnosis may be obtained from a patient's cells, such as, for example, from blood, urine, saliva, tissue biopsy, and autopsy material. The genomic DNA may be used directly for detection or may be amplified enzymatically by using PCR, for example, as described by Saiki et al., Nature, 324: 163-166 (1986), prior to analysis. RNA or cDNA may also be used for the same purpose. As an example, PCR primers complementary to the nucleic acid encoding a polypeptide of the present invention can be used to identify and analyze mutations. For example, deletions and insertions can be detected by a change in size of the amplified product in comparison to the normal genotype. Point mutations can be identified by hybridizing amplified DNA to radiolabeled RNA or alternatively, radiolabeled antisense DNA sequences. Perfectly matched sequences can be distinguished from mismatched duplexes by RNase A digestion or by differences in melting temperatures.

Cells may be engineered in vivo for expressing the polypeptide in vivo, for example, by procedures known in the art. As known in the art, a cell producing a retroviral particle containing RNA encoding the polypeptide of the present invention may be administered to a patient for the purpose of engineering cells in vivo and expressing the polypeptide in vivo. These and other methods for administering a polypeptide of the present invention by similar methods should be apparent to those skilled in the art from the teachings of the present invention. For example, the expression vehicle for engineering cells may be other than a retroviral particle, for example, an adenovirus, which may be used to engineer cells in vivo after combination with a suitable delivery vehicle.

Retroviruses from which the retroviral plasmid vectors hereinabove mentioned may be derived include, but are not limited to, Moloney murine leukemia virus, spleen necrosis virus, Rous sarcoma virus, Harvey sarcoma virus, avian leukosis virus, gibbon ape leukemia virus, human immunodeficiency virus, adenovirus (HIV), myeloproliferative sarcoma virus, and mammary tumor virus.

The nucleic acid sequence encoding the polypeptide of the present invention is under the control of a suitable promoter. Vectors of the invention include one or more promoters. Suitable promoters which may be employed include, but are not limited to, the retroviral long terminal repeat (LTR); the SV40 promoter; and the human cytomegalovirus (CMV) promoter described in Miller, et al., Biotechniques, Vol. 7, No. 9, 980-990 (1989), or any other homologous or heterologous promoter, for example, cellular promoters such as eukaryotic cellular promoters including, but not limited to, the histone, pol III, and β-actin promoters. Other viral promoters which may be employed include, but are not limited to, adenovirus promoters, for example, the adenoviral major late promoter; thymidine kinase (TK) promoters; and B19 parvovirus promoters.

Suitable promoters include, but are not limited to, the respiratory syncytial virus (RSV) promoter; inducible promoters, such as the MMT promoter, the metallothionein promoter; heat shock promoters; the albumin promoter; the ApoA1 promoter; human globin promoters; viral thymidine kinase promoters, such as the herpes simplex thymidine kinase promoter; retroviral LTRs (including the modified retroviral LTRs hereinabove described); the beta-actin promoter; and human growth hormone promoters. The promoter also may be the native promoter which controls the gene encoding the polypeptide. The selection of a suitable promoter will be apparent to those skilled in the art from the teachings contained herein.

A retroviral plasmid vector can be employed to transduce packaging cell lines to form producer cell lines. Examples of packaging cells which may be transfected include, but are not limited to, the PE501, PA317, PA12, T19-14X, VT-19-17-H2, CRE, CRIP, GP+E-86, GP+envAm12, and DAN cell lines as described in Miller, Human Gene Therapy, 1:5-14 (1990). The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include the nucleic acid sequence(s) encoding the polypeptides. Such retroviral vector particles then may be employed to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

Genetic testing based on DNA sequence differences may be achieved by detecting alterations in electrophoretic mobility of DNA fragments in gels run with or without denaturing agents. Small sequence deletions and insertions can be visualized by high resolution gel electrophoresis. DNA fragments of different sequences may be distinguished on denaturing formamide gradient gels in which the mobilities of different DNA fragments are retarded in the gel at different positions according to their specific melting or partial melting temperatures, for example, as described by Myers et al., Science, 230:1242 (1985).

Sequence changes at specific locations may also be revealed by nuclease protection assays, such as RNase and S1 protection or the chemical cleavage method as shown in Cotton et al., Proc. Natl. Acad. Sci., 85:4397-4401 (1985). Thus, the detection of a specific DNA sequence may be achieved by methods such as hybridization, RNase protection, chemical cleavage, direct DNA sequencing or the use of restriction enzymes, for example, Restriction Fragment Length Polymorphisms (RFLP) and Southern blotting of genomic DNA. In addition to more conventional gel-electrophoresis and DNA sequencing, mutations can also be detected by in situ analysis.

The present invention also relates to a diagnostic assay for detecting altered levels of Semaphorin 3F proteins in various tissues. An abnormal level of these proteins in muscle tissue may indicate abnormalities in neuromuscular connections which are found, for example, in the neuromuscular disorders discussed below. Assays used to detect protein levels in a host-derived sample are well-known to those of skill in the art and include radioimmunoassays, competitive-binding assays, Western Blot analysis, ELISA assays, “sandwich” assays, and other assays for the expression levels of the genes encoding the Semaphorin 3F proteins known in the art. Expression can be assayed by qualitatively or quantitatively measuring or estimating the level of Semaphorin 3F protein, or the level of mRNA encoding Semaphorin 3F protein, in a biological sample. Assays may be performed directly, for example, by determining or estimating absolute protein level or mRNA level, or relatively, by comparing the Semaphorin 3F protein or mRNA to a second biological sample. In performing these assays, the Semaphorin 3F protein or mRNA level in the first biological sample is measured or estimated and compared to a standard Semaphorin 3F protein level or mRNA level; suitable standards include second biological samples obtained from an individual not having the disorder of interest. Standards may be obtained by averaging levels of Semaphorin 3F in a population of individuals not having a disorder related to Semaphorin 3F expression. As will be appreciated in the art, once a standard Semaphorin 3F protein level or mRNA level is known, it can be used repeatedly as a standard for comparison.

An ELISA assay, for example, as described by Coligan, et al., Current Protocols in Immunology, 1(2), Chap. 6, (1991), utilizes an antibody prepared with specificity to a polypeptide antigen of the present invention. In addition, a reporter antibody is prepared against the monoclonal antibody. To the reporter antibody is attached a detectable reagent such as a radioactive tag, a fluorescent tag, or an enzymatic tag, e.g., a horseradish peroxidase. A sample is removed from a host and incubated on a solid support, e.g. a polystyrene dish, that binds the proteins in the sample. Any free protein binding sites on the dish are then covered by incubating with a non-specific protein, e.g., bovine serum albumin. Next, the specific antibody, e.g., a monoclonal antibody, is incubated in the dish, during which time the antibody attaches to any polypeptides of the present invention attached to the polystyrene dish. All unbound monoclonal antibody is washed out with buffer. The reporter antibody, for example, one linked to horseradish peroxidase is placed in the dish, resulting in the binding of the reporter antibody to any antibody bound to the protein of interest; unattached reporter antibody is then removed. Substrate, e.g., peroxidase, is then added to the dish, and the amount of signal produced color, e.g., developed in a given time period provides a measurement of the amount of a polypeptide of the present invention present in a given volume of patient sample when compared against a standard.

A competition assay may be employed wherein antibodies specific to a polypeptide of the present invention are attached to a solid support, and labeled Semaphorin 3F, along with a sample derived from the host, are passed over the solid support. The label can be detected and quantified, for example, by liquid scintillation chromatography, and the measurement can be correlated to the quantity of the polypeptide of interest present in the sample. A “sandwich” assay, similar to an ELISA assay, may be employed, wherein a polypeptide of the present invention is passed over a solid support and binds to antibody modules attached to the solid support. A second antibody is then bound to the polypeptide of interest. A third antibody, which is labeled and specific to the second antibody is then passed over the solid support and binds to the second antibody. The amount of antibody binding can be quantified; it correlates with the amount of the polypeptide of interest. See, for example, U.S. Pat. No. 4,376,110.

Biological samples of the invention can include any biological sample obtained from a subject, body fluid, cell line, tissue culture, or other source which contains Semaphorin 3F protein or mRNA. As indicated, biological samples include body fluids (such as sera, plasma, urine, synovial fluid, and spinal fluid) which may contain free Semaphorin 3F protein, cells such as endothelial, epithelial, or mesenchymal cells, tissues such as lung, vascular or muscle tissue, and any other cellular or tissue source found to express complete or mature Semaphorin 3F or related polypeptide. Methods for obtaining tissue biopsies and body fluids from mammals are well known in the art. Where the biological sample is to include mRNA, a tissue biopsy may provide the source.

Total cellular RNA can be isolated from a biological sample using any suitable technique such as the single-step guanidinium-thiocyanate-phenol-chloroform method described in Chomczynski and Sacchi, Anal. Biochem., 162:156-159 (1987). Levels of mRNA encoding the Semaphorin 3F protein are then assayed using any appropriate method. These include Northern blot analysis, S1 nuclease mapping, the polymerase chain reaction (PCR), reverse transcription in combination with the polymerase chain reaction (RT-PCR), and reverse transcription in combination with the ligase chain reaction (RT-LCR).

Assaying Semaphorin 3F protein levels in a biological sample can be performed using antibody-based techniques. For example, Semaphorin 3F protein expression in tissues can be studied with classical immunohistological methods, for example, Jalkanen, M., et al., J. Cell. Biol., 101:976-985 (1985); Jalkanen, M., et al., J. Cell. Biol., 105:3087-3096 (1987). Other antibody-based methods useful for detecting Semaphorin 3F protein gene expression include immunoassays, such as the enzyme linked immunosorbent assay (ELISA) and the radioimmunoassay (RIA). Suitable antibody assay labels are known in the art and include enzyme labels, such as glucose oxidase, radioisotopes, and fluorescent labels, such as fluorescein and rhodamine, and biotin.

In addition to assaying Semaphorin 3F protein levels in a biological sample obtained from an individual, Semaphorin 3F protein can also be detected in vivo by imaging. Antibody labels or markers for in vivo imaging of Semaphorin 3F protein include those detectable by X-radiography, NMR, or ESR. For X-radiography, suitable labels include radioisotopes such as barium or cesium, which emit detectable radiation but are not overtly harmful to a subject. Suitable markers for NMR and ESR include those with a detectable characteristic spin, such as deuterium, which may be incorporated into the antibody by labeling of nutrients for the relevant hybridoma.

A Semaphorin 3F protein-specific antibody or antibody fragment which has been labeled with an appropriate detectable imaging moiety, such as a radioisotope, a radio-opaque substance, or a material detectable by nuclear magnetic resonance, is introduced, for example, parenterally, subcutaneously, or intraperitoneally, into a subject to be examined or being treated for a muscle disorder or into a subject to be examined or being treated for a neuromuscular defect caused by injury. It will be understood in the art that the size of the subject and the imaging system used will determine the quantity of imaging moiety needed to produce diagnostic images. The labeled antibody or antibody fragment will then accumulate at the location of cells which contain Semaphorin 3F protein. As an illustration, in vivo tumor imaging is described in Burchiel et al., ed., Chapter 13, Tumor Imaging: The Radiochemical Detection of Cancer, Masson Publishing, Inc. (1982).

Therapeutic Uses of Semaphorin 3F Related Molecules, Agonists, Antagonists, and Host Cells

Molecules of the invention and fragments and variants thereof may be used in diagnosing, determining the prognosis for, preventing, treating, and developing treatments for any disorder (or defect) characterized by abnormal levels of neuromuscular connectivity. Semaphorin 3F related nucleic acids, vectors, polypeptides, agonists, antagonists, or host cells may be administered to a patient afflicted with such a disorder. A gene therapy approach may be applied to treat disorders that are caused by defective Semaphorin 3F. Disclosure herein of sequences of the invention permits the detection of defective Semaphorin 3F related genes, and the replacement thereof with normal or corrective genes. Defective genes may be detected in in vitro diagnostic assays, and by comparison of the sequences of the invention with that of a gene derived from a patient suspected of harboring a defect.

Molecules of the invention, such as, for example, recombinant Semaphorin 3F or antagonists thereof, may have distinct effects on different muscle cell types and may affect the same cell type differently under different conditions. For example, under conditions wherein Semaphorin 3F promotes the formation of certain neuromuscular connections, recombinant Semaphorin 3F or related molecules may be used to stimulate neuromuscular regeneration. Under conditions wherein Semaphorin 3F inhibits the formation of certain neuromuscular connections, antagonists of Semaphorin 3F may be used to stimulate neuromuscular regeneration. Suitable antagonists of Semaphorin 3F are described herein, and may include inhibitory antibodies, small molecule inhibitors, antisense oligonucleotides, RNAi molecules, and soluble receptors or receptor fragments, such as the extracellular domain of a receptor.

Disease Applications

The molecules of the invention, including Semaphorin 3F and related molecules, as well as antagonists thereof, are useful for treating, preventing, diagnosing, and/or determining a prognosis for muscle disorders or diseases. As described above, muscular disorders or muscular diseases encompass muscular and neuromuscular disorders, some of which are characterized by a destabilization or improper organization of the plasma membrane of specific cell types and include muscular dystrophies (MDs). MDs are a group of genetic degenerative myopathies characterized by weakness and muscle atrophy without nervous system involvement. The three main types of MD are pseudohypertrophic (Duchenne, Becker), limb-girdle (LGMD), and facioscapulohumeral. Several muscular dystrophies and muscular atrophies are characterized by a breakdown of the muscle cell membrane, i.e., they are characterized by leaky membranes resulting from a mutation in dystrophin, some of which can be treated by compensatory overexpression of utrophin. Muscular disorders which can be treated by semaphorin 3A further encompasses Welander distal myopathy (WDM), Hereditary Distal Myopathy, Benign Congenital Hypotonia, Central Core disease, Nemaline Myopathy, and Myotubular (centronuclear) myopathy, as well as muscle wasting, sarcopenia, and muscular atrophies. Non-limiting examples of muscular atrophies are those resulting from AIDS-related wasting, from denervation (loss of contact by the muscle with its nerve) due to nerve trauma; degenerative, metabolic or inflammatory neuropathy (e.g., Guillian Barre syndrome), peripheral neuropathy, and damage to nerves caused by environmental toxins or drugs; muscle atrophies that result from denervation due to a motor neuronopathy, including adult motor neuron disease, Amyotrophic Lateral Sclerosis (ALS or Lou Gehrig's disease); infantile and juvenile spinal muscular atrophies, and autoimmune motor neuropathy with multifocal conduction block; muscle atrophies that result from chronic disuse, including disuse atrophy stemming from conditions including, but not limited to: paralysis due to stroke, spinal cord injury; skeletal immobilization due to trauma (such as fracture, sprain or dislocation) or prolonged bed rest; and muscle atrophies resulting from metabolic stress or nutritional insufficiency, including, but not limited to, the cachexia of cancer and other chronic illnesses, fasting orrhabdomyolysis, and endocrine disorders such as, but not limited to, disorders of the thyroid gland and diabetes.

The molecules of the invention may act as repellents or attractants in interactions between muscle and nerve cells that are required for the formation of functional neuromuscular junctions, for example, during the renervation of injured or diseased tissue. Hence, the molecules of the invention are useful in the treatment of all neuromuscular disorders or abnormalities, including defects due to injury, where the inhibition or promotion of neuromuscular regeneration is desired.

In an embodiment the molecules of the invention are used to treat, prevent, diagnose and/or determine a prognosis for various forms of degenerative or atrophic disorders, including, but not limited to, diabetic myopathy, Charcot Marie Tooth syndrome, chronic inflammatory demyelinating polyradiculoneuropathy, conditions caused by toxins such as lead, mercury or certain chemotherapeutic agents such as taxanes and platinums, Amyotrophic Lateral Sclerosis (ALS or Lou Gehrig's disease), poliomyelitis, post-polio syndrome, nerve entrapment syndromes such as carpal tunnel syndrome and ulnar compression, and HIV-associated neuropathy.

Additionally, the molecules of the invention may be employed not only as therapeutic molecules as described herein, but additionally as research tools in elucidating the biology of muscle disorders and any other disease.

Antibodies

Antibodies specific to Semaphorin 3F are suitable for use as therapeutic agents in the present invention and can be raised against the intact Semaphorin 3F protein or an antigenic polypeptide fragment thereof. The protein or fragment may be presented with or without a carrier protein, such as an albumin, to an animal, such as a rabbit or mouse). In general, polypeptide fragments are sufficiently immunogenic to produce a satisfactory immune response without a carrier if they are at least about 25 amino acids in length.

Antibodies of the invention include polyclonal and monoclonal antibody preparations, as well as preparations including hybrid antibodies, altered antibodies, chimeric antibodies and, humanized antibodies, as well as hybrid (chimeric) antibody molecules (see, for example, Winter et al., Nature 349:293-299 (1991)); and U.S. Pat. No. 4,816,567); F(ab′)₂ and F(ab) fragments; Fv molecules (noncovalent heterodimers, see, for example, Inbar et al., Proc. Natl. Acad. Sci. 69:2659-2662 (1972)); and Ehrlich et al. (1980) Biochem 19:4091-4096); single chain Fv molecules (sFv) (see, e.g., Huston et al., Proc. Natl. Acad. Sci. 85:5879-5883 (1980)); dimeric and trimeric antibody fragment constructs; minibodies (see, e.g., Pack et al., Biochem. 31:1579-1584 (1992); Cumber et al., J. Immunology 149B:120-126 (1992)); humanized antibody molecules (see, e.g., Riechmann et al., Nature 332:323-327 (1988); Verhoeyan et al., Science 239:1534-1536 (1988)); heteroconjugate and bispecific antibodies (see, e.g., U.S. Pat. No. 6,010,902 and U.S. Patent Appln. 2002/0155604); and any functional fragments obtained from such molecules, wherein such fragments retain specific binding.

Methods of making monoclonal and polyclonal antibodies are known in the art. Monoclonal antibodies are generally antibodies having a homogeneous antibody population. The term is not limited regarding the species or source of the antibody, nor is it intended to be limited by the manner in which it is made. The term encompasses whole immunoglobulins. Polyclonal antibodies are typically generated by immunizing a suitable animal, such as a mouse, rat, rabbit, sheep or goat, with an antigen of interest, such as a stem cell transformed with a gene encoding an antigen. In order to enhance immunogenicity, the antigen can be linked to a carrier prior to immunization. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Furthermore, the antigen may be conjugated to a bacterial toxoid, such as a toxoid from diphtheria, tetanus, cholera, etc., in order to enhance the immunogenicity thereof.

In addition, techniques developed for the production of chimeric antibodies (Morrison et al., Proc. Natl. Acad. Sci., 81:851-855 (1984); Neuberger et al., Nature, 312:604-608 (1984); Takeda et al., Nature, 314:452-454 (1985)) by splicing genes from a mouse antibody molecule of appropriate antigen specificity together with genes from a human antibody molecule of appropriate biological activity can be used. Chimeric antibodies, which are antibodies in which different portions are derived from different animal species, such as those having a variable region derived from a murine monoclonal antibody and a human immunoglobulin constant region, for example, humanized antibodies, and insertion/deletions relating to cdr and framework regions, are suitable for use in the invention.

The invention also includes humanized antibodies, i.e., those with mostly human immunoglobulin sequences. Humanized antibodies of the invention generally refer to non-human immunoglobulins that have been modified to incorporate portions of human sequences. A humanized antibody may include a human antibody that contains entirely human immunoglobulin sequences.

The antibodies of the invention may be prepared by any of a variety of methods. For example, cells expressing an Semaphorin 3F protein or an antigenic fragment thereof can be administered to an animal in order to induce the production of sera containing polyclonal antibodies. A preparation of Semaphorin 3F protein can be prepared and purified to render it substantially free of natural contaminants, and the preparation introduced into an animal in order to produce polyclonal antisera with specific binding activity.

Antibodies of the invention specifically bind to their respective antigen(s); they may display high avidity and/or high affinity to a specific polypeptide, or more accurately, to an epitope of an antigen. Antibodies of the invention may bind to one epitope, or to more than one epitope. They may display different affinities and/or avidities to different epitopes on one or more molecules. When an antibody binds more strongly to one epitope than to another, adjusting the binding conditions can, in some instances, result in antibody binding almost exclusively to the specific epitope and not to any other epitopes on the same polypeptide, and not to a polypeptide that does not comprise the epitope.

The invention also provides monoclonal antibodies and Semaphorin 3F protein binding fragments thereof. Monoclonal antibodies of the invention can be prepared using hybridoma technology, for example, Kohler et al., Nature, 256:495 (1975); Kohler et al., Eur. J. Immunol., 6:511 (1976); Kohler et. al., Eur. J. Immunol., 6:292 (1976); Hammerling et al., in: Monoclonal Antibodies and T-Cell Hybridomas, Elsevier, N.Y., (1981) pp.563-681. In general, such procedures involve immunizing an animal (for example, a mouse) with an Semaphorin 3F protein antigen or with an Semaphorin 3F protein-expressing cell. Suitable cells can be recognized by their capacity to bind anti-Semaphorin 3F protein antibody. Such cells may be cultured in any suitable tissue culture medium; for example, in Earle's modified Eagle's medium supplemented with 10% fetal bovine serum (inactivated at about 56° C.), and supplemented with about 10 grams/liter of nonessential amino acids, about 1,000 U/ml of penicillin, and about 100 μg/ml of streptomycin. The splenocytes of such mice are extracted and fused with a suitable myeloma cell line. Any suitable myeloma cell line may be employed in accordance with the present invention; e.g., the parent myeloma cell line (SP20), available from the American Type Culture Collection (ATCC), Manassas, Va. After fusion, the resulting hybridoma cells are selectively maintained in HAT medium, and then cloned by limiting dilution, for example, as described by Wands et al., Gastroenterology, 80:225-232 (1981).

Alternatively, antibodies capable of binding to the Semaphorin 3F protein antigen may be produced in a two-step procedure through the use of anti-idiotypic antibodies. Such a method makes use of the fact that antibodies are themselves antigens, and that, therefore, it is possible to obtain an antibody which binds to a second antibody. In accordance with this method, specific antibodies are used to immunize an animal such as a mouse. The splenocytes of such an animal are then used to produce hybridoma cells, and the hybridoma cells are screened to identify clones which produce an antibody whose ability to bind to the specific antibody can be blocked by the antigen. Such antibodies comprise anti-idiotypic antibodies to the Semaphorin 3F protein-specific antibody and can be used to immunize an animal to induce formation of further specific antibodies.

It will be appreciated that Fab and F(ab′)₂ and other fragments of the antibodies of the present invention may be used according to the methods disclosed herein. Such fragments are typically produced by proteolytic cleavage, using enzymes such as papain (to produce Fab fragments) or pepsin (to produce F(ab′)₂ fragments). Alternatively, Semaphorin 3F protein-binding fragments can be produced through the application of recombinant DNA technology or through synthetic chemistry. Humanized chimeric monoclonal antibodies are suitable for in vivo use of anti-Semaphorin 3F in humans. Such humanized antibodies can be produced using genetic constructs derived from hybridoma cells producing the monoclonal antibodies described above. Methods for producing chimeric antibodies are known in the art. See, for review, Morrison, Science, 229:1202 (1985); Oi et al., BioTechniques, 4:214 (1986); Cabilly et al., U.S. Pat. No. 4,816,567; Taniguchi et al., EP 0 171 496; Morrison et al., EP 0 173 494; Neuberger et al., WO 8601533; Robinson et al., WO 8702671; Boulianne et al., Nature, 312:643 (1984); Neuberger et al., Nature, 314:268 (1985).

Kits

The present invention provides kits that can be used in the above methods. In an embodiment, the invention provides a diagnostic kit comprising an isolated polypeptide of the invention, a carrier, and a reporter for detecting the polypeptide. In an embodiment, the invention provides an isolated nucleic acid molecule of the invention, a reporter for detecting the nucleic acid molecule and/or its complement, and a vehicle.

In an embodiment, a kit comprises an antibody of the invention, for example, a purified antibody, in one or more containers. In an embodiment, the kits of the invention contain a substantially isolated polypeptide comprising an epitope which is specifically immunoreactive with an antibody included in the kit. The kits of the invention may also comprise a control antibody which does not react with the polypeptide of interest. They may further comprise one or more carriers for the antibody and yet further comprise a reporter for detecting antibody binding.

In an embodiment, the kits of the present invention comprise a means for detecting the binding of an antibody to a polypeptide of interest, i.e., a reporter. For example, the antibody may be conjugated to a detectable substrate such as a fluorescent compound, an enzymatic substrate, a radioactive compound or a luminescent compound, or a second antibody which recognizes the first antibody may be conjugated to a detectable substrate).

In an embodiment, the kit is a diagnostic kit for use in screening serum containing antibodies specific against Semaphorin 3F or related molecules. Such a kit may include a control antibody that does not react with the polypeptide of interest. Such a kit may include a substantially isolated polypeptide antigen comprising an epitope which is specifically immunoreactive with at least one anti-polypeptide antigen antibody. Further, such a kit includes means for detecting the binding of the antibody to the antigen. The antibody may be conjugated to a fluorescent compound, such as fluorescein or rhodamine, which can be detected by flow cytometry. In an embodiment, the kit may include a recombinantly produced or chemically synthesized polypeptide antigen. The polypeptide antigen of the kit may also be attached to a solid support.

In a further embodiment, the detecting means of the above-described kit includes a solid support to which said polypeptide antigen is attached. Such a kit may also include a non-attached reporter-labeled anti-human antibody. In this embodiment, binding of the antibody to the polypeptide antigen can be detected by binding of the said reporter-labeled antibody.

In an additional embodiment, the invention includes a diagnostic kit for use in screening serum containing antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In an embodiment, the antibody is attached to a solid support. In an embodiment, the antibody is a monoclonal antibody. The detecting means of the kit may include a second, labeled monoclonal antibody. Alternatively, or in addition, the detecting means may include a labeled, competing antigen.

In a diagnostic configuration, test serum is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or calorimetric substrate.

The solid surface reagent may be prepared by known techniques for attaching protein material to solid support material, such as polymeric beads, dip sticks, 96-well plates, and/or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with a biotinylated antigen.

The description in this specification is put forth to provide those of ordinary skill in the art with a complete disclosure of how to make and how to use the present invention, and is not intended to limit the scope of what the inventors regard as their invention, nor is it intended to represent that the experiments set forth are all or the only experiments performed.

While the present invention is described with reference to specific embodiments thereof, it should be understood by those skilled in the art that various changes may be made and equivalents may be substituted without departing from the true spirit and scope of the invention. In addition, many modifications can be made to adapt to a particular situation, material, composition of matter, process, process step or steps, to the objective, spirit, and scope of the present invention. All such modifications are intended to be within the scope of the claims appended hereto.

Unless defined otherwise, the meanings of all technical and scientific terms used herein are those commonly understood by one of ordinary skill in the art to which this invention belongs.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges including either or both of the upper and lower limits of the range, unless specifically excluded from the stated range.

It must be noted that, as used herein and in the appended claims, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

Further, all numbers expressing quantities of ingredients, reaction conditions, % purity, polypeptide and polynucleotide lengths, and so forth, used in the specification, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement. The specification is most thoroughly understood in light of the cited references, all of which are hereby incorporated by reference in their entireties.

### EXAMPLE

### Example 1

### Identification of Semaphorin 3F as an Effector of Skeletal Muscle Cells

A. A High-Throughput Screening Method Using an Impedance Assay

To identify factors that directly regulate skeletal muscle cell activity, factors were screened for their ability to affect the impedance of human primary skeletal muscle cells. Cultured cells are electrically active and their electrical resistance can be measured by growing them in assay wells equipped with microelectronic sensors. A commercially available cell-electrode impedance measuring system is the real-time, cell electronic system (RT-CES™ System) from ACEA Bioscience, Inc., (San Diego, Calif.). The system comprises a multiwell tissue culture plate with integrated microelectronic sensors coupled to an impedance analyzer, which in turn is coupled to a computer. It has been described in U.S. Patent Application Publication US 2004/0152067 A1. When a cell or the fluid in the well connects to electrodes in the sensor, the impedance analyzer measures the impedance resulting from alternating voltage applied across the electrodes. Cells seeded in the wells attach to the electrodes and change the resistance between the electrodes. Changes in the electrical resistance of the cells caused, for instance, by stimulation of a signaling pathway by binding of a ligand to its receptor, are measured as changes in impedance (Abassi et al., J. Immuno. Meth., 292: 195-205 (2004); Giaever et al., Proc. Nat'l. Acad. Sci., 81: 3761-3764 (1984)).

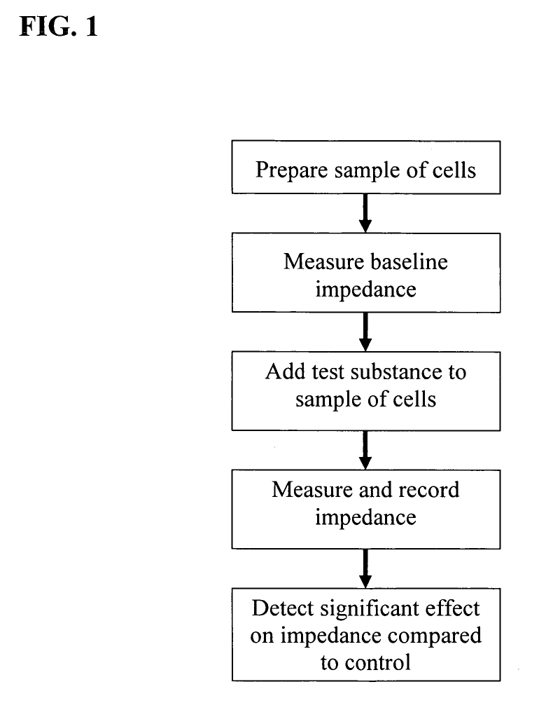

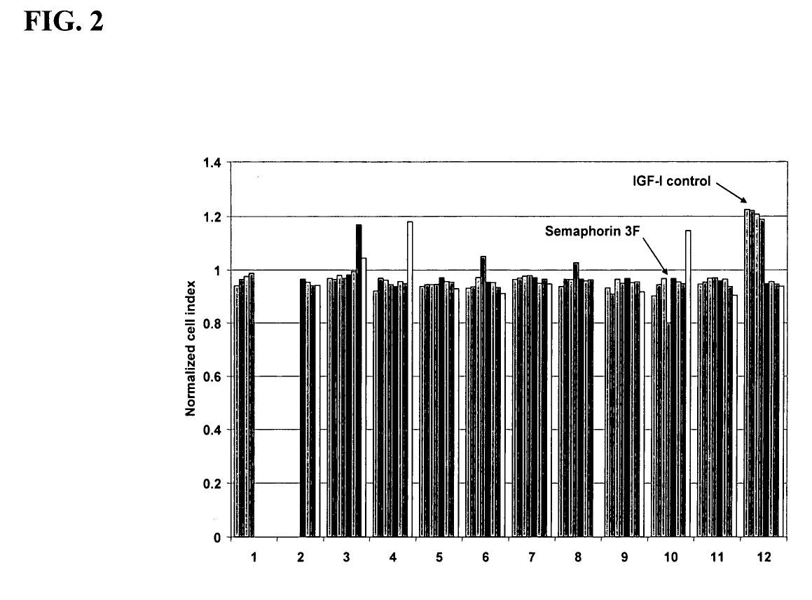

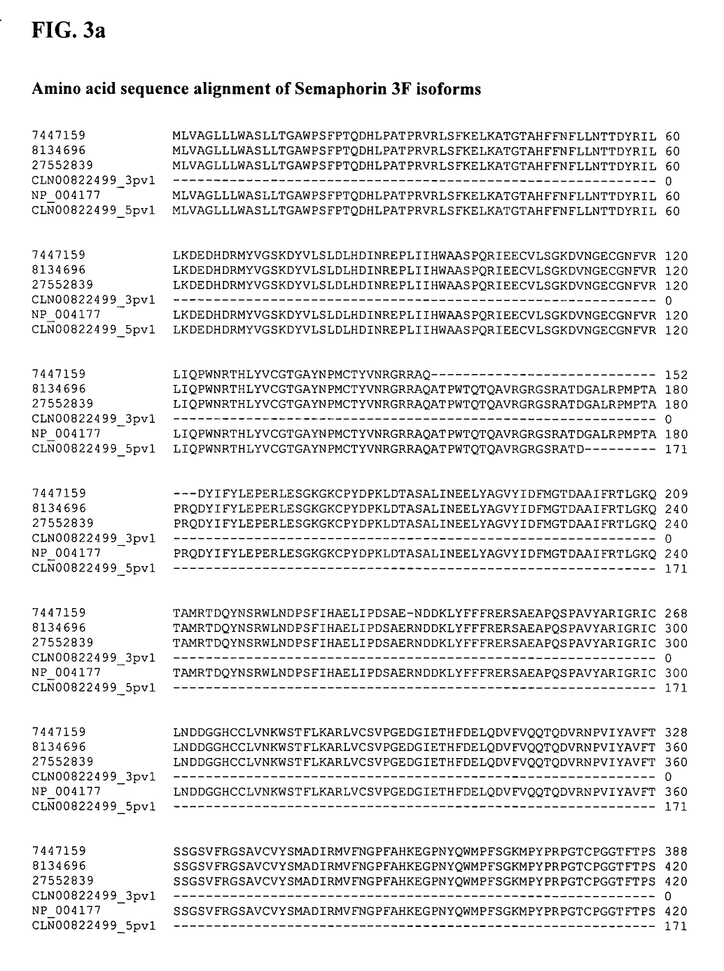

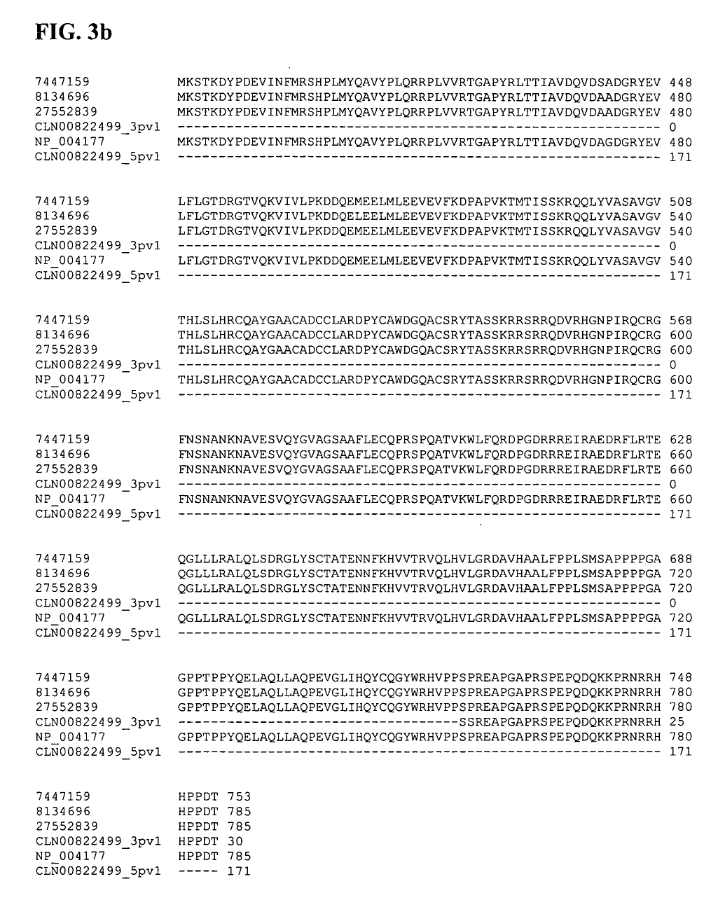

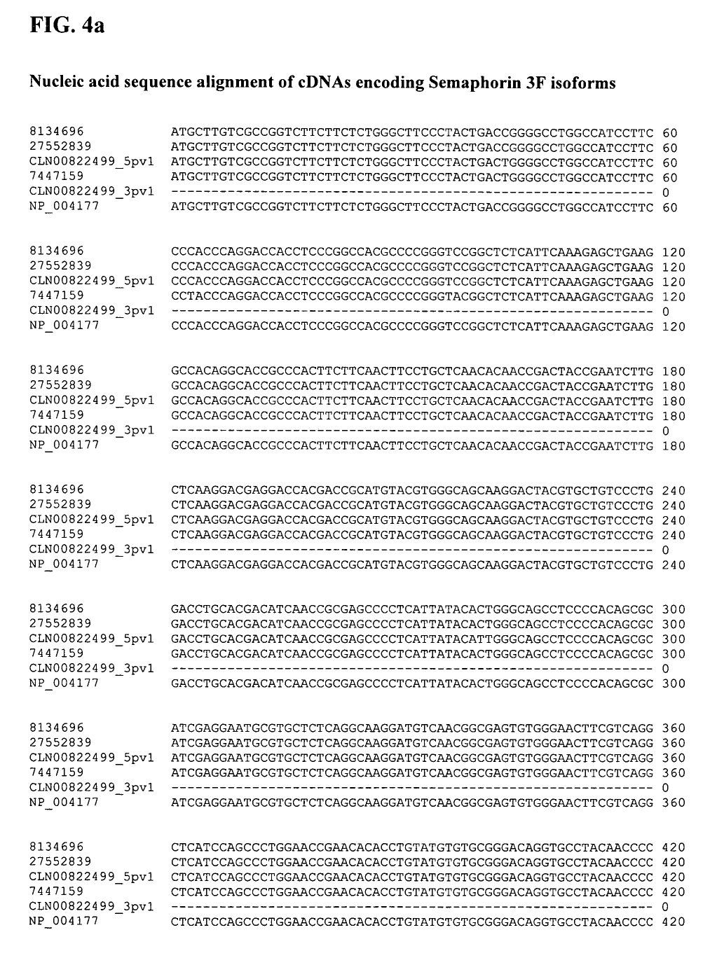

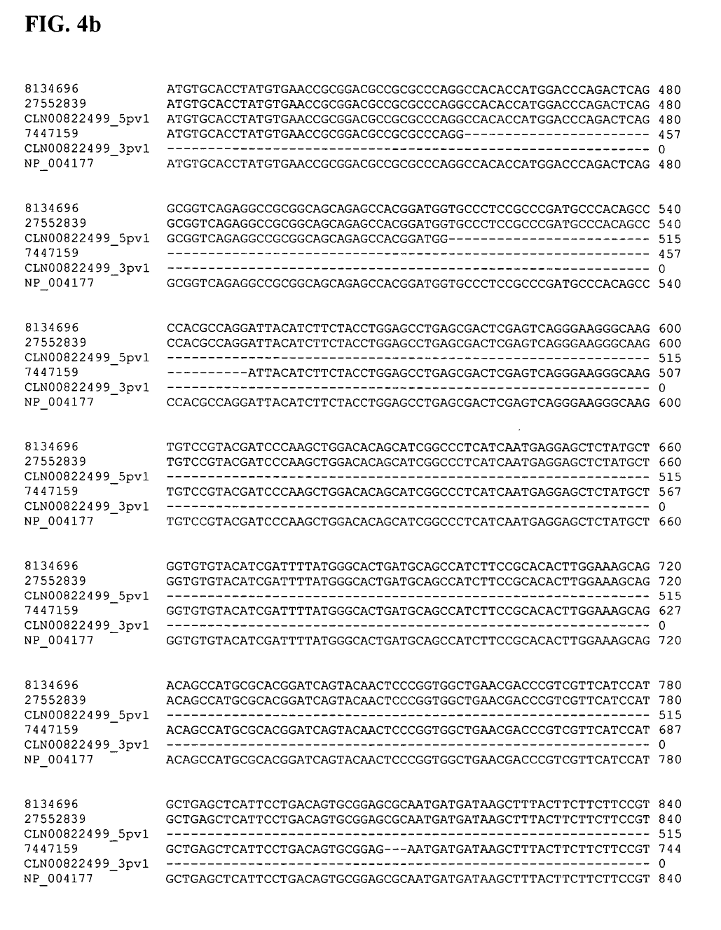

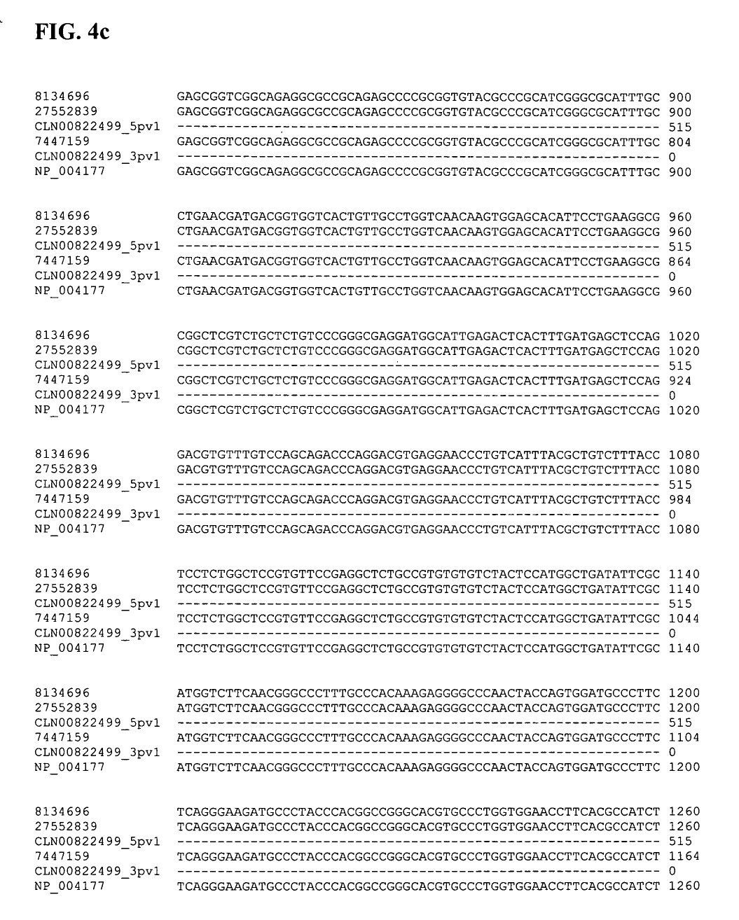

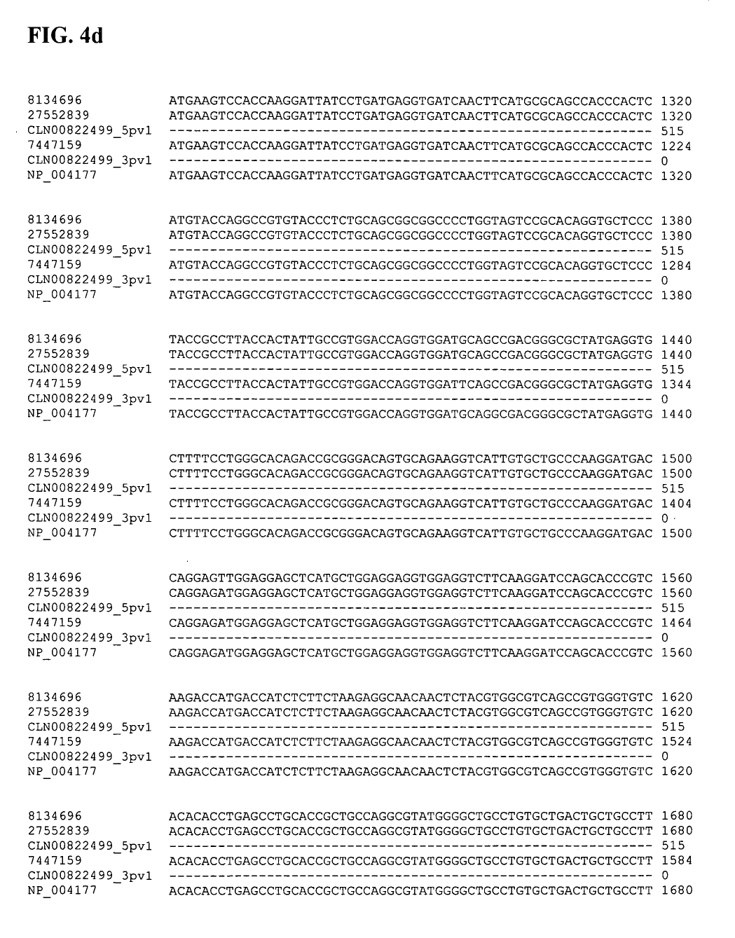

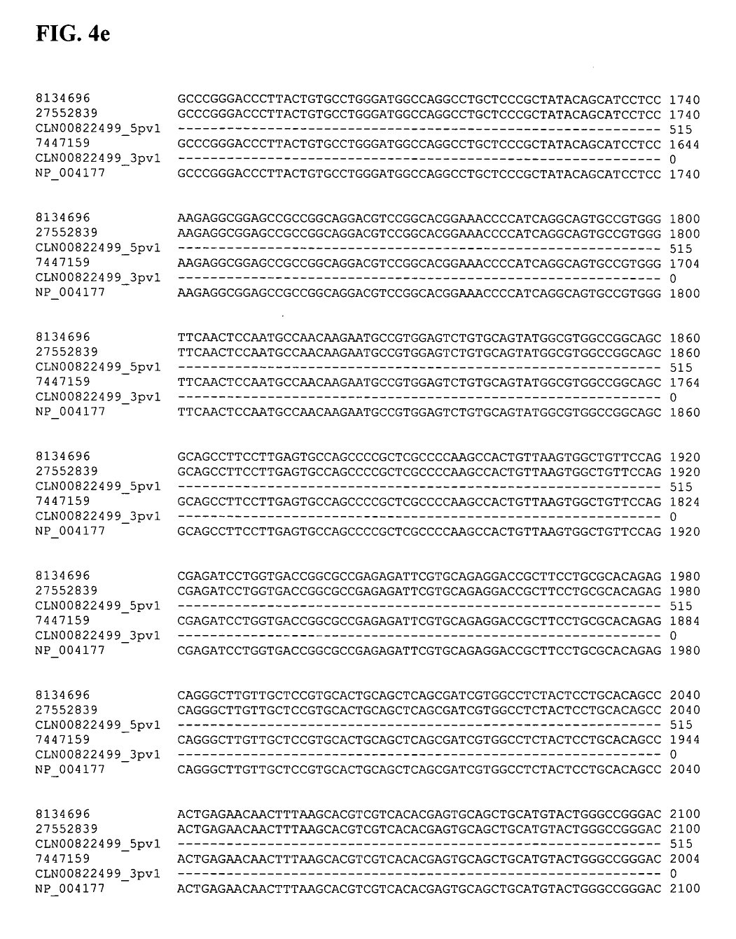

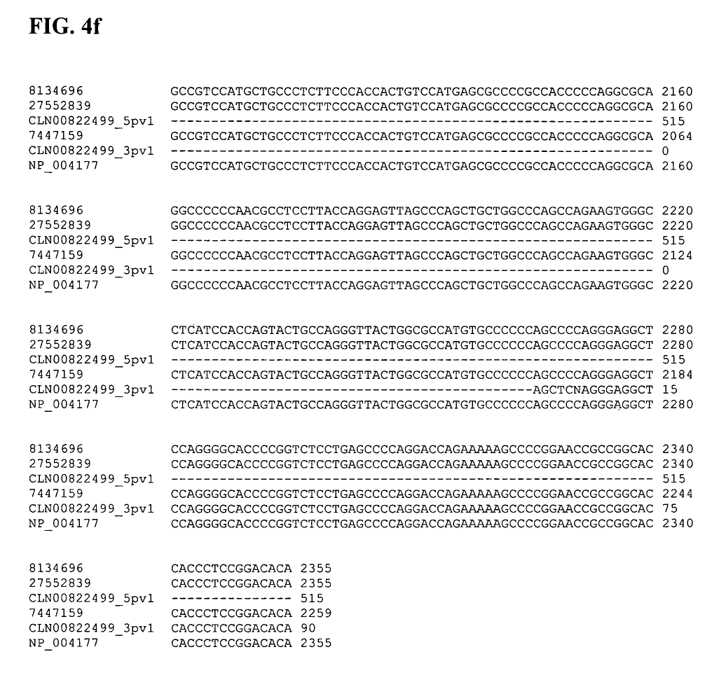

Impedance-measuring systems have been used for monitoring cell proliferation, cell toxicity, and receptor-ligand interaction. The RT-CES System calculates a normalized change in impedance resulting from the cells adhering to the microelectrodes and provides a baseline reading. The electrical response of the cells upon ligand addition can be measured in real time by adding the ligands to be tested to the culture well (Abassi et al., J. Immunol., Meth. 292: 195-205 (2004)). The overall steps of operating the real-time commercially available cell-electrode impedance-measuring system (RT-CESTM System) from ACEA Bioscience, Inc. (San Diego, Calif.) are depicted in FIG. 1.

B. Identification of Semaphorin 3F as a Direct Effector of Skeletal Muscle Cells in the Impedance Assay

The impedance assay was performed using an RT-CES™ 16X device (ACEA Bioscience, Inc., San Diego, Calif.) substantially according to manufacturer's instructions, except where otherwise indicated. Briefly, each well of each 96 well plate was coated with 0.1% gelatin, and about 3×10⁴ primary human skeletal muscle cells (Cambrex, East Rutherford, N.J.) were seeded into each well in a growth medium for these cells (DMEM supplemented with 25 mM HEPES, 10% fetal calf serum, 2 mM glutamine, 0.5% chick embryo extract, 100 U/ml penicillin, 100 μg/ml streptomycin, and 0.25 μg/ml amphotericin B; the medium and supplements were also obtained from Cambrex). The cells were permitted to attach to the plate, and were then incubated overnight at 37° C. in 5% CO₂.

To test a panel of agents for their effects on cell impedance/cell index, the baseline impedance was established after the overnight incubation. For this purpose, the growth medium was replaced with serum-free medium and the cells were incubated for another six hr. The baseline was established by measuring impedance at two-minute intervals over a four-minute period. After establishing a baseline, the serum-free medium was replaced with medium comprising the test agents to be screened. One agent was tested per well and the impedance of each well was measured every two minutes for a total of 30 minutes. The cell index, as a measure of the changing impedance, was calculated by the RT-CES™ 16X device software. The results of the measurements obtained after 30 minutes are show in FIG. 2. Insulin-like growth factor I (IGF-I) (Cat# 291-G1; R&D Systems, Minneapolis, Minn.), at a concentration of 10 nM, was used as an external positive control. Columns 1-12 and rows A-H refer to the grid of wells in the 96 well plate. Semaphorin 3F (arrow) is contained in well D10. Well H4 contains the internal positive control insulin growth factor-I (IGF-I). The internal positive control IGF-I was provided in form of conditioned medium that was produced in parallel and by the same methods as the test agents. Betacellulin is contained in well G3. Wells 12A-D contain the external positive control 10 nM IGF-I. No data are shown with respect to wells 1E-H and 2A-D. These results show that Semaphorin 3F induced a significant change in cell index in human primary skeletal muscle cells, as measured by this impedance assay.

### Tables

TABLE 1
Protein and Nucleotide Sequence Identification Numbers

| | SEQ. | | | |
|---|---|---|---|---|
| | ID NO. | SEQ. ID NO. | SEQ. ID NO. | |
| FP ID | N1 | P1 | N0 | Clone ID |
| HG1021598 | 1 | 5 | 9 | 7447159 |
| HG1021599 | 2 | 6 | 10 | NP_004177 |
| HG1021600 | 3 | 7 | 11 | 8134696 |
| HG1021601 | 4 | 8 | 12 | 27552839 |

TABLE 2
Annotation and Pfam Domains of Semaphorin 3F Sequences

| | | Pfam | Pfam | |
|---|---|---|---|---|
| FP ID | Clone ID | Domains | Coords | Annotation |
| HG1021598 | 7447159 | Sema | 57-497 | semaphorin III |
| | | ig | 587-648 | family homolog |
| | | | | [Homo sapiens] |
| HG1021599 | NP_004177 | Sema | 57-529 | Semaphorin 3F |
| | | ig | 619-680 | [Homo sapiens] |
| HG1021600 | 8134696 | Sema | 57-529 | Semaphorin-3F |
| | | ig | 619-680 | precursor |
| | | | | (Semaphorin IV) |
| | | | | (Sema IV) (Sema |
| | | | | III/F) |
| | | | | [Homo sapiens] |
| HG1021601 | 27552839 | Sema | 57-529 | Semaphorin 3F |
| | | ig | 619-680 | [Homo sapiens] |

TABLE 3
Amino Acid Coordinates of Secreted Semaphorin 3F Polypeptides

| | Signal | Mature | | | |
|---|---|---|---|---|---|
| | Peptide | Protein | Non-TM | Alternate Signal | Alternate Mature |
| FP ID | Coords | Coords | Coords | Peptide Coords | Protein Coords |
| HG1021598 | 1-18 | 19-753 | 19-753 | (1-15) (1-16) (1-17) | (15-753) (16-753) (17-753) |
| HG1021599 | 1-18 | 19-785 | 19-785 | (1-15) (1-16) (1-17) | (15-785) (16-785) (17-785) |
| HG1021600 | 1-18 | 19-785 | 19-785 | (1-15) (1-16) (1-17) | (15-785) (16-785) (17-785) |
| HG1021601 | 1-18 | 19-785 | 19-785 | (1-15) (1-16) (1-17) | (15-785) (16-785) (17-785) |

### Sequence Listing

Applicants include a Sequence Listing provided in both electronic format and in paper format.

| | |
|---|---|
| ATGCTTGTCGCCGGTCTTCTTCTCTGGGCTTCCCTA | SEQ. ID NO. 1 |
| CTGACTGGGGCCTGGCCATCCTTCCCTACCCAGGAC | |
| CACCTCCCGGCCACGCCCCGGGTACGGCTCTCATTC | |
| AAGAGCTGAAGGCCACAGGCACCGCCCACTTCTTCA | |
| ACTTCCTGCTCAACACAACCGACTACCGAATCTTGC | |
| TCAAGGACGAGGACCACGACCGCATGTACGTGGGCA | |
| GCAAGGACTACGTGCTGTCCCTGGACCTGCACGACA | |
| TCAACCGCGAGCCCCTCATTATACACTGGGCAGCCT | |
| CCCCACAGCGCATCGAGGAATGCGTGCTCTCAGGCA | |
| AGGATGTCAACGGCGAGTGTGGGAACTTCGTCAGGC | |
| TCATCCAGCCCTGGAACCGAACACACCTGTATGTGT | |
| GCGGGACAGGTGCCTACAACCCCATGTGCACCTATG | |
| TGAACCGCGGACGCCGCGCCCAGGATTACATCTTCT | |
| ACCTGGAGCCTGAGCGACTCGAGTCAGGGAAGGGCA | |
| AGTGTCCGTACGATCCCAAGCTGGACACAGCATCGG | |
| CCCTCATCAATGAGGAGCTCTATGCTGGTGTGTACA | |
| TCGATTTTATGGGCACTGATGCAGCCATCTTCCGCA | |
| CACTTGGAAAGCAGACAGCCATGCGCACGGATCAGT | |
| ACAACTCCCGGTGGCTGAACGACCCGTCGTTCATCC | |
| ATGCTGAGCTCATTCCTGACAGTGCGGAGAATGATG | |
| ATAAGCTTTACTTCTTCTTCCGTGAGCGGTCGGCAG | |
| AGGCGCCGCAGAGCCCCGCGGTGTACGCCCGCATCG | |
| GGCGCATTTGCCTGAACGATGACGGTGGTCACTGTT | |
| GCCTGGTCAACAAGTGGAGCACATTCCTGAAGGCGC | |
| GGCTCGTCTGCTCTGTCCCGGGCGAGGATGGCATTG | |
| AGACTCACTTTGATGAGCTCCAGGACGTGTTTGTCC | |
| AGCAGACCCAGGACGTGAGGAACCCTGTCATTTACG | |
| CTGTCTTTACCTCCTCTGGCTCCGTGTTCCGAGGCT | |
| CTGCCGTGTGTGTCTACTCCATGGCTGATATTCGCA | |
| TGGTCTTCAACGGGCCCTTTGCCCACAAAGAGGGGC | |
| CCAACTACCAGTGGATGCCCTTCTCAGGGAAGATGC | |
| CCTACCCACGGCCGGGCACGTGCCCTGGTGGAACCT | |
| TCACGCCATCTATGAAGTCCACCAAGGATTATCCTG | |
| ATGAGGTGATCAACTTCATGCGCAGCCACCCACTCA | |
| TGTACCAGGCCGTGTACCCTCTGCAGCGGCGGCCCC | |
| TGGTAGTCCGCACAGGTGCTCCCTACCGCCTTACCA | |
| CTATTGCCGTGGACCAGGTGGATTCAGCCGACGGGC | |
| GCTATGAGGTGCTTTTCCTGGGCACAGACCGCGGGA | |
| CAGTGCAGAAGGTCATTGTGCTGCCCAAGGATGACC | |
| AGGAGATGGAGGAGCTCATGCTGGAGGAGGTGGAGG | |
| TCTTCAAGGATCCAGCACCCGTCAAGACCATGACCA | |
| TCTCTTCTAAGAGGCAACAACTCTACGTGGCGTCAG | |
| CCGTGGGTGTCACACACCTGAGCCTGCACCGCTGCC | |
| AGGCGTATGGGGCTGCCTGTGCTGACTGCTGCCTTG | |
| CCCGGGACCCTTACTGTGCCTGGGATGGCCAGGCCT | |
| GCTCCCGCTATACAGCATCCTCCAAGAGGCGGAGCC | |
| GCCGGCAGGACGTCCGGCACGGAAACCCCATCAGGC | |
| AGTGCCGTGGGTTCAACTCCAATGCCAACAAGAATG | |
| CCGTGGAGTCTGTGCAGTATGGCGTGGCCGGCAGCG | |
| CAGCCTTCCTTGAGTGCCAGCCCCGCTCGCCCCAAG | |
| CCACTGTTAAGTGGCTGTTCCAGCGAGATCCTGGTG | |
| ACCGGCGCCGAGAGATTCGTGCAGAGGACCGCTTCC | |
| TGCGCACAGAGCAGGGCTTGTTGCTCCGTGCACTGC | |
| AGCTCAGCGATCGTGGCCTCTACTCCTGCACAGCCA | |
| CTGAGAACAACTTTAAGCACGTCGTCACACGAGTGC | |
| AGCTGCATGTACTGGGCCGGGACGCCGTCCATGCTG | |
| CCCTCTTCCCACCACTGTCCATGAGCGCCCCGCCAC | |
| CCCCAGGCGCAGGCCCCCCAACGCCTCCTTACCAGG | |
| AGTTAGCCCAGCTGCTGGCCCAGCCAGAAGTGGGCC | |
| TCATCCACCAGTACTGCCAGGGTTACTGGCGCCATG | |
| TGCCCCCCAGCCCCAGGGAGGCTCCAGGGGCACCCC | |
| GGTCTCCTGAGCCCCAGGACCAGAAAAAGCCCCGGA | |
| ACCGCCGGCACCACCCTCCGGACACA | |
| ATCCTTGTCGCCGGTCTTCTTCTCTGGGCTTCCCTA | SEQ. ID NO. 2 |
| CTGACCGGGGCCTGGCCATCCTTCCCCACCCAGGAC | |
| CACCTCCCGGCCACGCCCCGGGTCCGGCTCTCATTC | |
| AAAGAGCTGAAGGCCACAGGCACCGCCCACTTCTTC | |
| AACTTCCTGCTCAACACAACCGACTACCGAATCTTG | |
| CTCAAGGACGAGGACCACGACCGCATGTACGTGGGC | |
| AGCAAGGACTACGTGCTGTCCCTGGACCTGCACGAC | |
| ATCAACCGCGAGCCCCTCATTATACACTGGGCAGCC | |
| TCCCCACAGCGCATCGAGGAATGCGTGCTCTCAGGC | |
| AAGGATGTCAACGGCGAGTGTGGGAACTTCGTCAGG | |
| CTCATCCAGCCCTGGAACCGAACACACCTGTATGTG | |
| TGCGGGACAGGTGCCTACAACCCCATGTGCACCTAT | |
| GTGAACCGCGGACGCCGCGCCCAGGCCACACCATGG | |
| ACCCAGACTCAGGCGGTCAGAGGCCGCGGCAGCAGA | |
| GCCACGGATGGTGCCCTCCGCCCGATGCCCACAGCC | |
| CCACGCCAGGATTACATCTTCTACCTGGAGCCTGAG | |
| CGACTCGAGTCAGGGAAGGGCAAGTGTCCGTACGAT | |
| CCCAAGCTGGACACAGCATCGGCCCTCATCAATGAG | |
| GAGCTCTATGCTGGTGTGTACATCGATTTTATGGGC | |
| ACTGATGCAGCCATCTTCCGCACACTTGGAAAGCAG | |
| ACAGCCATGCGCACGGATCAGTACAACTCCCGGTGG | |
| CTGAACGACCCGTCGTTCATCCATGCTGAGCTCATT | |
| CCTGACAGTGCGGAGCGCAATGATGATAAGCTTTAC | |
| TTCTTCTTCCGTGAGCGGTCGGCAGAGGCGCCGCAG | |
| AGCCCCGCGGTGTACGCCCGCATCGGGCGCATTTGC | |
| CTGAACGATGACGGTGGTCACTGTTGCCTGGTCAAC | |
| AAGTGGAGCACATTCCTGAAGGCGCGGCTCGTCTGC | |
| TCTGTCCCGGGCGAGGATGGCATTGAGACTCACTTT | |
| GATGAGCTCCAGGACGTGTTTGTCCAGCAGACCCAG | |
| GACGTGAGGAACCCTGTCATTTACGCTGTCTTTACC | |
| TCCTCTGGCTCGGTGTTCCGAGGCTCTGCCGTGTGT | |
| GTCTACTCCATGGCTGATATTCGCATGGTCTTCAAC | |
| GGGCCCTTTGCCCACAAAGAGGGGCCCAACTAGCAG | |
| TGGATGCCCTTCTCAGGGAAGATCCCCTACCCACGG | |
| CCGGGCACGTGCCCTGGTGGAACCTTCACGCCATCT | |
| ATGAAGTCCACCAAGGATTATCCTGATGAGGTGATC | |
| AACTTCATGCGCAGCCACCCACTCATGTACCAGGCC | |
| GTGTACGCTCTGCAGCGGCGGCCCCTGGTAGTCCGC | |
| ACAGGTGCTCCCTACCGCCTTACCACTATTGCCGTG | |
| GACCAGGTGGATGCAGGCGACGGGCGCTATGAGGTG | |
| CTTTTCCTGGGCACAGACCGCGGGACAGTGCAGAAG | |
| GTCATTGTGCTGCCCAAGGATGACCAGGAGATGGAG | |
| GAGCTCATGCTGGAGGAGGTGGAGGTCTTCAAGGAT | |
| CCAGCACCCGTCAAGACCATGACCATCTCTTCTAAG | |
| AGGCAACAACTCTACGTGGCGTCAGCCGTGGGTGTC | |
| ACACACCTGAGCCTGCACCGCTGCCAGGCGTATGGG | |
| GCTGCCTGTGCTGACTGCTGCCTTGCCCGGGACCCT | |
| TACTGTGCCTGGGATGGCCAGGCCTGCTCCCGCTAT | |
| ACAGCATCCTCCAAGAGGCGGAGCCGCCGGCAGGAC | |
| GTCCGGCACGGAAACCCCATCAGGCAGTGCCGTGGG | |
| TTCAACTCCAATGCCAACAAGAATGCCGTGGAGTCT | |
| GTGCAGTATGGCGTGGCCGGCAGCGCAGCCTTCCTT | |
| GAGTGCCAGCCCCGCTCGCCCCAAGCCACTGTTAAG | |
| TGGCTGTTCCAGCGAGATCCTGGTGACCGGCGCCGA | |
| GAGATTCGTGCAGAGGACCGCTTCCTGCGCACAGAG | |
| CAGGGCTTGTTGCTCCGTGCACTGCAGCTCAGCGAT | |
| CGTGGCCTCTACTCCTGCACAGCCACTGAGAACAAC | |
| TTTAAGCACGTCGTCACACGAGTGCAGCTGCATGTA | |
| CTGGGCCGGGACGCCGTCCATGCTGCCCTCTTCCCA | |
| CCACTGTCCATGAGCGCCCCGCCACCCCCAGGCGCA | |
| GGCCCCCCAACGCCTCCTTACCAGGAGTTAGCCCAG | |
| CTGCTGGCCCAGCCAGAAGTGGGCCTCATCCACCAG | |
| TACTGCCAGGGTTACTGGCGCCATGTGCCCCCCAGC | |
| CCCAGGGAGGCTCCAGGGGCACCCCGGTCTCCTGAG | |
| CCCCAGGACCAGAAAAAGCCCCGGAACCGCCGGCAC | |
| CACCCTCCGGACACA | |
| ATGCTTGTCGCCGGTCTTCTTCTCTGGGCTTCCCTA | SEQ. ID NO. 3 |
| CTGACCGGGGCCTGGCCATCCTTCCCCACCCAGGAC | |
| CACCTCCCGGCCACGCCCCGGGTCCGGCTCTCATTC | |
| AAAGAGCTGAAGGCCACAGGCACCGCCCACTTCTTC | |
| AACTTCCTGCTCAACACAACCGACTACCGAATCTTG | |
| CTCAAGGACGAGGACCACGACCGCATGTACGTGGGC | |
| AGCAAGGACTACGTGCTGTCCCTGGACCTGCACGAC | |
| ATCAACCGCGAGCCCCTCATTATACACTGGGCAGCC | |
| TCCCCACAGCGCATCGAGGAATGCGTGCTCTCAGGC | |
| AAGGATGTCAACGGCGAGTGTGGGAACTTCGTCAGG | |
| CTCATCCAGCCCTGGAACCGAACACACCTGTATGTG | |
| TGCGGGACAGGTGCCTACAACCCCATGTGCACCTAT | |
| GTGAACCGCGGACGCCGCGCCCAGGCCACACCATGG | |
| ACCCAGACTCAGGCGGTCAGAGGCCGCGGCAGCAGA | |
| GCCACGGATGGTGCCCTCCGCCCGATGCCCACAGCC | |
| CCACGCCAGGATTACATCTTCTACCTGGAGCCTGAG | |
| CGACTCGAGTCAGGGAAGGGCAAGTGTCCGTACGAT | |
| CCCAAGCTGGACACAGCATCGGCCCTCATCAATGAG | |
| GAGCTCTATGCTGGTGTGTACATCGATTTTATGGGC | |
| ACTGATGCAGCCATCTTCCGCACACTTGGAAAGCAG | |
| ACAGCCATGCGCACGGATCAGTACAACTCCCGGTGG | |
| CTGAACGACCCGTCGTTCATCCATGCTGAGCTCATT | |
| CCTGACAGTGCGGAGCGCAATGATGATAAGCTTTAC | |
| TTCTTCTTCCGTGAGCGGTCGGCAGAGGCGCCGCAG | |
| AGCCCCGCGGTGTACGCCCGCATCGGGCGCATTTGC | |
| CTGAACGATGACGGTGGTCACTGTTGCCTGGTCAAC | |
| AAGTGGAGCACATTCCTGAAGGCGCGGCTCGTCTGC | |
| TCTGTCCCGGGCGAGGATGGCATTGAGACTCACTTT | |
| GATGAGCTCCAGGACGTGTTTGTCCAGCAGACCCAG | |
| GACGTGAGGAACCCTGTCATTTACGCTGTCTTTACC | |
| TCCTCTGGCTCCGTGTTCCGAGGCTCTGCCGTGTGT | |
| GTCTACTCCATGGCTGATATTCGCATGGTCTTCAAC | |
| GGGCCCTTTGCCCACAAAGAGGGGCCCAACTACCAG | |
| TGGATGCCCTTCTCAGGGAAGATGCCCTACCCACGG | |
| CCGGGCACGTGCCCTGGTGGAACCTTCACGCCATCT | |
| ATGAAGTCCACCAAGGATTATCCTGATGAGGTGATC | |
| AACTTCATGCGCAGCCACCCACTCATGTACCAGGCC | |
| GTGTACCCTCTGCAGCGGCGGCCCCTGGTAGTCCGC | |
| ACAGGTGCTCCCTACCGCCTTACCACTATTGCCGTG | |
| GACCAGGTGGATGCAGCCGACGGGCGCTATGAGGTG | |
| CTTTTCCTGGGCACAGACCGCGGGACAGTGCAGAAG | |
| GTCATTGTGCTGCCCAAGGATGACCAGGAGTTGGAG | |
| GAGCTCATGCTGGAGGAGGTGGAGGTCTTCAAGGAT | |
| CCAGCACCCGTCAAGACCATGACCATCTCTTCTAAG | |
| AGGCAACAACTCTACGTGGCGTCAGCCGTGGGTGTC | |
| ACACACCTGAGCCTGCACCGCTGCCAGGCGTATGGG | |
| GCTGCCTGTGCTGACTGCTGCCTTGCCCGGGACCCT | |
| TACTGTGCCTGGGATGGCCAGGCCTGCTCCCGCTAT | |
| ACAGCATCCTCCAAGAGGCGGAGCCGCCGGCAGGAC | |
| GTCCGGCACGGAAACCCCATCAGGCAGTGCCGTGGG | |
| TTCAACTCCAATGCCAACAAGAATGCCGTGGAGTCT | |
| GTGCAGTATGGCGTGGCCGGCAGCGCAGCCTTCCTT | |
| GAGTGCCAGCCCCGCTCGCCCCAAGCCACTGTTAAG | |
| TGGCTGTTCCAGCGAGATCCTGGTGACCGGCGCCGA | |
| GAGATTCGTGCAGAGGACCGCTTCCTGCGCACAGAG | |
| CAGGGCTTGTTGCTCCGTGCACTGCAGCTCAGCGAT | |
| CGTGGCCTCTACTCCTGCACAGCCACTGAGAACAAC | |
| TTTAAGCACGTCGTCACACGAGTGCAGCTGCATGTA | |
| CTGGGCCGGGACGCCGTCCATGCTGCCCTCTTCCCA | |
| CCACTGTCCATGAGCGCCCCGCCACCCCCAGGCGCA | |
| GGCCCCCCAACGCCTCCTTACCAGGAGTTAGCCCAG | |
| CTGCTGGCCCAGCCAGAAGTGGGCCTCATCCACCAG | |
| TACTGCCAGGGTTACTGGCGCCATGTGCCCCCCAGC | |
| CCCAGGGAGGCTCCAGGGGCACCCCGGTCTCCTGAG | |
| CCCCAGGACCAGAAAAAGCCCCGGAACCGCCGGCAC | |
| CACCCTCCGGACACA | |
| ATGCTTGTCGCCGGTCTTCTTCTCTGGGCTTCCCTA | SEQ. ID NO. 4 |
| CTGACCGGGGCCTGGCCATCCTTCCCCACCCAGGAC | |
| CACCTCCCGGCCACGCCCCGGGTCCGGCTCTCATTC | |
| AAAGAGCTGAAGGCCACAGGCACCGCCCACTTCTTC | |
| AACTTCCTGCTCAACACAACCGACTACCGAATCTTG | |
| CTCAAGGACGAGGACCACGACCGCATGTACGTGGGC | |
| AGCAAGGACTACGTGCTGTCCCTGGACCTGCACGAC | |
| ATCAACCGCGAGCCCCTCATTATACACTGGGCAGCC | |
| TCCCCACAGCGCATCGAGGAATGCGTGCTCTCAGGC | |
| AAGGATGTCAACGGCGAGTGTGGGAACTTCGTCAGG | |
| CTCATCCAGCCCTGGAACCGAACACACCTGTATGTG | |
| TGCGGGACAGGTGCCTACAACCCCATGTGCACCTAT | |
| GTGAACCGCGGACGCCGCGCCCAGGCCACACCATGG | |
| ACCCAGACTCAGGCGGTCAGAGGCCGCGGCAGCAGA | |
| GCCACGGATGGTGCCCTCCGCCCGATGCCCACAGCC | |
| CCACGCCAGGATTACATCTTCTACCTGGAGCCTGAG | |
| CGACTCGAGTCAGGGAAGGGCAAGTGTCCGTACGAT | |
| CCCAAGCTGGACACAGCATCGGCCCTCATCAATGAG | |
| GAGCTCTATGCTGGTGTGTACATCGATTTTATGGGC | |
| ACTGATGCAGCCATCTTCCGCACACTTGGAAAGCAG | |
| ACAGCCATGCGCACGGATCAGTACAACTCCCGGTGG | |
| CTGAACGACCCGTCGTTCATCCATGCTGAGCTCATT | |
| CCTGACAGTGCGGAGCGCAATGATGATAAGCTTTAC | |
| TTCTTCTTCCGTGAGCGGTCGGCAGAGGCGCCGCAG | |
| AGCCCCGCGGTGTACGCCCGCATCGGGCGCATTTGC | |
| CTGAACGATGACGGTGGTCACTGTTGCCTGGTCAAC | |
| AAGTGGAGCACATTCCTGAAGGCGCGGCTCGTCTGC | |
| TCTGTCCCGGGCGAGGATGGCATTGAGACTCACTTT | |
| GATGAGCTCCAGGACGTGTTTGTCCAGCAGACCCAG | |
| GACGTGAGGAACCCTGTCATTTACGCTGTCTTTACC | |
| TCCTCTGGCTCCGTGTTCCGAGGCTCTGCCGTGTGT | |
| GTCTACTCCATGGCTGATATTCGCATGGTCTTCAAC | |
| GGGCCCTTTGCCCACAAAGAGGGGCCCAACTACCAG | |
| TGGATGCCCTTCTCAGGGAAGATGCCCTACCCACGG | |
| CCGGGCACGTGCCCTGGTGGAACCTTCACGCCATCT | |
| ATGAAGTCCACCAAGGATTATCCTGATGAGGTGATC | |
| AACTTCATGCGCAGCCACCCACTCATGTACCAGGCC | |
| GTGTACCCTCTGCAGCGGCGGCCCCTGGTAGTCCGC | |
| ACAGGTGCTCCCTACCGCCTTACCACTATTGCCGTG | |
| GACCAGGTGGATGCAGCCGACGGGCGCTATGAGGTG | |
| CTTTTCCTGGGCACAGACCGCGGGACAGTGCAGAAG | |
| GTCATTGTGCTGCCCAAGGATGACCAGGAGATGGAG | |
| GAGCTCATGCTGGAGGAGGTGGAGGTCTTCAAGGAT | |
| CCAGCACCCGTCAAGACCATGACCATCTCTTCTAAG | |
| AGGCAACAACTCTACGTGGCGTCAGCCGTGGGTGTC | |
| ACACACCTGAGCCTGCACCGCTGCCAGGCGTATGGG | |
| GCTGCCTGTGCTGACTGCTGCCTTGCCCGGGACCCT | |
| TACTGTGCCTGGGATGGCCAGGCCTGCTCCCGCTAT | |
| ACAGCATCCTCCAAGAGGCGGAGCCGCCGGCAGGAC | |
| GTCCGGCACGGAAACCCCATCAGGCAGTGCCGTGGG | |
| TTCAACTCCAATGCCAACAAGAATGCCGTGGAGTCT | |
| GTGCAGTATGGCGTGGCCGGCAGCGCAGCCTTCCTT | |
| GAGTGCCAGCCCCGCTCGCCCCAAGCCACTGTTAAG | |
| TGGCTGTTCCAGCGAGATCCTGGTGACCGGCGCCGA | |
| GAGATTCGTGCAGAGGACCGCTTCCTGCGCACAGAG | |
| CAGGGCTTGTTGCTCCGTGCACTGCAGCTCAGCGAT | |
| CGTGGCCTCTACTCCTGCACAGCCACTGAGAACAAC | |
| TTTAAGCACGTCGTCACACGAGTGCAGCTGCATGTA | |
| CTGGGCCGGGACGCCGTCCATGCTGCCCTCTTCCCA | |
| CCACTGTCCATGAGCGCCCCGCCACCCCCAGGCGCA | |
| GGCCCCCCAACGCCTCCTTACCAGGAGTTAGCCCAG | |
| CTGCTGGCCCAGCCAGAAGTGGGCCTCATCCACCAG | |
| TACTGCCAGGGTTACTGGCGCCATGTGCCCCCCAGC | |
| CCCAGGGAGGCTCCAGGGGCACCCCGGTCTCCTGAG | |
| CCCCAGGACCAGAAAAAGCCCCGGAACCGCCGGCAC | |
| CACCCTCCGGACACA | |
| MLVAGLLLWASLLTGAWPSFPTQDHLPATPRVRLSF | SEQ. ID NO. 5 |
| KELKATGTAHFFNFLLNTTDYRILLKDEDHDRMYVG | |
| SKDYVLSLDLHDINREPLIIHWAASPQRIEECVLSG | |
| KDVNGECGNFVRLIQPWNRTHLYVCGTGAYNPMCTY | |
| VNRGRRAQDYIFYLEPERLESGKGKCPYDPKLDTAS | |
| ALINEELYAGVYIDFMGTDAAIFRTLGKQTANRTDQ | |
| YNSRWLNDPSFIHAELIPDSAENDDKLYFFERERSA | |
| EAPQSPAVYARIGRICLNDDGGHCCLVNKWSTFLKA | |
| RLVCSVPGEDGIETHFDELQDVFVQQTQDVRNPVIY | |
| AVFTSSGSVFRGSAVCVYSMADIRMVFNGPFAHKEG | |
| PNYQWMPFSGKMPYPRPGTCPGGTFTPSMKSTKDYP | |
| DEVINFMRSHPLMYQAVYPLQRRPLVVRTGAPYRLT | |
| TIAVDQVDSADGRYEVLFLGTDRGTVQKVIVLPKDD | |
| QEMEELMLEEVEVEKDPAPVKTMTISSKRQQLYVAS | |
| AVGVTHLSLHRCQAYGAACADCCLARDPYCAWDGQA | |
| CSRYTASSKRRSRRQDVRHGNPIRQCRGFNSNANKN | |
| AVESVQYGVAGSAAELECQPRSPQATVKWLEQRDPG | |
| DRRREIRAEDRFLRTEQGLLLRALQLSDRGLYSCTA | |
| TENNFKHVVTRVQLHVLGRDAVHAALFPPLSMSAPP | |
| PPGAGPPTPPYQELAQLLAQPEVGLIHQYCQGYWRH | |
| VPPSPREAPGAPRSPEPQDQKKPRNRRHHPPDT | |
| MLVAGLLLWASLLTGAWPSFPTQDHLPATPRVRLSF | SEQ. ID NO. 6 |
| KELKATGTAHFFNFLLNTTDYRILLKDEDHDRMYVG | |
| SKDYVLSLDLHDINREPLIIHWAASPQRIEECVLSG | |
| KDVNGECGNFVRLIQPWNRTHLYVCGTGAYNPMCTY | |
| VNRGRRAQATPWTQTQAVRGRGSRATDGALRPMPTA | |
| PRQDYIFYLEPERLESGKGKCPYDPKLDTASALINE | |
| ELYAGVYIDFMGTDAAIFRTLGKQTAMRTDQYNSRW | |
| LNDPSFIHAELIPDSAERNDDKLYEFFRERSAEAPQ | |
| SPAVYARIGRICLNDDGGHCCLVNKWSTFLKARLVC | |
| SVPGEDGIETHEDELQDVFVQQTQDVRNPVIYAVFT | |
| SSGSVFRGSAVCVYSMADIRMVFNGPFAHKEGPNYQ | |
| WMPFSGKMPYPRPGTCPGGTFTPSMKSTKDYPDEVI | |
| NFMRSHPLMYQAVYPLQRRPLVVRTGAPYRLTTIAV | |
| DQVDAGDGRYEVLFLGTDRGTVQKVIVLPKDDQEME | |
| ELMLEEVEVFKDPAPVKTMTISSKRQQLYVASAVGV | |
| THLSLHRCQAYGAACADCCLARDPYCAWDGQACSRY | |
| TASSKRRSRRQDVRHGNPTRQCRGENSNANKNAVES | |
| VQYGVAGSAAFLECQPRSPQATVKWLFQRDPGDRRR | |
| EIRAEDRELRTEQGLLLRALQLSDRGLYSCTATENN | |
| EKHVVTRVQLHVLGRDAVHAALFPPLSMSAPPPPGA | |
| GPPTPPYQELAQLLAQPEVGLIHQYCQGYWRHVPPS | |
| PREAPGAPRSPEPQDQKKPRNRRHHPPDT | |
| MLVAGLLLWASLLTGAWPSFPTQDHLPATPRVRLSF | SEQ. ID NO. 7 |
| KELKATGTAHFFNELLNTTDYRILLKDEDHDRMYVG | |
| SKDYVLSLDLHDTNREPLIIHWAASPQRIEECVLSG | |
| KDVNGECGNEVRLIQPWNRTHLYVCGTGAYNPMCTY | |
| VNRGRRAQATPWTQTQAVRGRGSRATDGALRPMPTA | |
| PRQDYIEYLEPERLESGKGKCPYDPKLDTASALINE | |
| ELYAGVYIDFMGTDAAIERTLGKQTAMRTDQYNSRW | |
| LNDPSETHAELIPDSAERNDDKLYEEERERSAEAPQ | |
| SPAVYARIGRICLNDDGGHCCLVNKWSTELKARLVC | |
| SVPGEDGTETHFDELQDVEVQQTQDVRNPVIYAVET | |
| SSGSVPRGSAVCVYSMADIRMVFNGPFAHKEGPNYQ | |
| WMPFSGKMPYPRPGTCPGGTFTPSMKSTKDYPDEVI | |
| NFMRSHPLMYQAVYPLQRRPLVVRTGAPYRLTTIAV | |
| DQVDAADGRYEVLELGTDRGTVQKVIVLPKDDQELE | |
| ELMLEEVEVFKDPAPVKTMTISSKRQQLYVASAVGV | |
| THLSLHRCQAYGAACADCCLARDPYCAWDGQACSRY | |
| TASSKRRSRRQDVRHGNPIRQCRGFNSNANKNAVES | |
| VQYGVAGSAAFLECQPRSPQATVKWLEQRDPGDRRR | |
| EIRAEDRFLRTEQGLLLRALQLSDRGLYSCTATENN | |
| FKHVVTRVQLHVLGRDAVHAALFPPLSMSAPPPPGA | |
| GPPTPPYQELAQLLAQPEVGLIHQYCQGYWRHVPPS | |
| PREAPGAPRSPEPQDQKKPRNRRHHPPDT | |
| MLVAGLLLWASLLTGAWPSFPTQDHLPATPRVRLSE | SEQ. ID NO. 8 |
| KELKATGTAHFFNFLLNTTDYRILLKDEDHDRMYVG | |
| SKDYVLSLDLHDINREPLIIHWAASPQRIEECVLSG | |
| KDVNGECGNFVRLIQPWNRTHLYVCGTGAYNPMCTY | |
| VNRGRRAQATPWTQTQAVRGRGSRATDGALRPMPTA | |
| PRQDYIEYLEPERLESGKGKCPYDPKLDTASALINE | |
| ELYAGVYIDFMGTDAAIFRTLGKQTAMRTDQYNSRW | |
| LNDPSFIHAELIPDSAERNDDKLYFFERERSAEAPQ | |
| SPAVYARIGRICLNDDGGHCCLVNKWSTFLKARLVC | |
| SVPGEDGIETHFDELQDVEVQQTQDVRNPVIYAVET | |
| SSGSVERGSAVCVYSMADIRMVENGPFAHKEGPNYQ | |
| WMPFSGKMPYPRPGTCPGGTFTPSMKSTKDYPDEVI | |
| NEMRSHPLMYQAVYPLQRRPLVVRTGAPYRLITIAV | |
| DQVDAADGRYEVLELGTDRGTVQKVIVLPKDDQEME | |
| ELMLEEVEVEKDPAPVKTMTISSKRQQLYVASAVGV | |
| THLSLHRCQAYGAACADCCLARDPYCAWDGQACSRY | |
| IASSKRRSRRQDVRHGNPIRQCRGFNSNANKNAVES | |
| VQYGVAGSAAELECQPRSPQATVKWLFQRDPGDRRR | |
| EIRAEDRELRTEQGLLLRALQLSDRGLYSCTAIENN | |
| FKHVVTRVQLHVLGRDAVHAALFPPLSMSAPPPPGA | |
| GPPTPPYQELAQLLAQPEVGLIHQYCQGYWRHVPPS | |
| PREAPGAPRSPEPQDQKKPRNRRHHPPDT | |
| ATGCTTGTCGCCGGTCTTCTTCTCTGGGCTTCCCTA | SEQ. ID NO. 9 |
| CTGACTGGGGCCTGGCCATCCTTCCCIACCCAGGAC | |
| CACCTCCCGGCCACGCCCCGGGTACGGCTCTCATTC | |
| AAAGAGCTGAAGGCCACAGGCACCGCCCACTTCTTC | |
| AACTTCCTGCTCAACACAACCGACTACCGAATCTTG | |
| CTCAAGGACGACGACCACGACCGCATGTACGTGGGC | |
| AGCAAGGACTACGTGCTGTCCCTGGACCTGCACGAC | |
| ATCAACCGCGAGCCCCTCATTATACACTGGGCAGCC | |
| TCCCCACAGCGCATCGAGGAATGCGTGCTCTCAGGC | |
| AAGGATGTCAACGGCGAGTGTGGGAACTTCGTCAGG | |
| CTCATCCAGCCCTGGAACCGAACACACCTGTATGTG | |
| TGCGGGACAGGTGCCTACAACCCCATGTGCACCTAT | |
| GTGAACCGCGGACGCCGCGCCCAGGATTACATCTTC | |
| TACCTGGAGCCTGAGCGACTCGAGTCAGGGAAGGGC | |
| AAGTGTCCGTACGATCCCAAGCTGGACACAGCATCG | |
| GCCCTCATCAATGAGGAGCTCTATGCTGGTGTGTAC | |
| ATCGATTTTATGGGCACTGATGCAGCCATCTTCCGC | |
| ACACTTGGAAAGCAGACAGCCATGCGCACGGATCAG | |
| TACAACTCCCGGTGGCTGAACGACCCGTCGTTCATC | |
| CATGCTGAGCTCATTCCTGACAGTGCGGAGAATGAT | |
| GATAAGCTTTACTTCTTCTTCCGTGAGCGGTCGGCA | |
| GAGGCGCCGCAGAGCCCCGCGGTGTACGCCCGCATC | |
| GGGCGCATTTGCCTGAACGATGACGGTGGTCACTGT | |
| TGCCTGGTCAACAAGTGGAGCACATTCCTGAAGGCG | |
| CGGCTCGTCTGCTCTGTCCCGGGCGAGGATGGCATT | |
| GAGACTCACTTTGATGAGCTCCAGGACGTGTTTGTC | |
| CAGCAGACCCAGGACGTGAGGAACCCTGTCATTTAC | |
| GCTGTCTTTACCTCCTCTGGCTCCGTGTTCCGAGGC | |
| TCTGCCGTGTGTGTCTACTCCATGGCTGATATTCGC | |
| ATGGTCTTCAACGGGCCCTTTGCCCACAAAGAGGGG | |
| CCCAACTACCAGTGGATGCCCTTCTCAGGGAAGATG | |
| CCCTACCCACGGCCGGGCACGTGCCCTGGTGGAACC | |
| TTCACGCCATCTATGAAGTCCACCAAGGATTATCCT | |
| GATGAGGTGATCAACTTCATGCGCAGCCACCCACTC | |
| ATGTACCAGGCCGTGTACCCTCTGCAGCGGCGGCCC | |
| CTGGTAGTCCGCACAGGTGCTCCCTACCGCCTTACC | |
| ACTATTGCCGTGGACCAGGTGGATTCAGCCGACGGG | |
| CGCTATGAGGTGCTTTTCCTGGGCACAGACCGCGGG | |
| ACAGTGCAGAAGGTCATTGTGCTGCCCAAGGATGAC | |
| CAGGAGATGGAGGAGCTCATGCTGGAGGAGGTGGAG | |
| GTCTTCAAGGATCCAGCACCCGTCAAGACCATGACC | |
| ATCTCTTCTAAGAGGCAACAACTCTACGTGGCGTCA | |
| GCCGTGGGTGTCACACACCTGAGCCTGCACCGCTGC | |
| CAGGCGTATGGGGCTGCCTGTGCTGACTGCTGCCTT | |
| GCCCGGGACCCTTACTGTGCCTGGGATGGCCAGGCC | |
| TGCTCCCGCTATACAGCATCCTCCAAGAGGCGGAGC | |
| CGCCGGCAGGACGTCCGGCACGGAAACCCCATCAGG | |
| CAGTGCCGTGGGTTCAACTCCAATGCCAACAAGAAT | |
| GCCGTGGAGTCTGTGCAGTATGGCGTGGCCGGCAGC | |
| GCAGCCTTCCTTGAGTGCCAGCCGCGCTCGCCCCAA | |
| GCCACTGTTAAGTGGCTGTTCCAGCGAGATCCTGGT | |
| GACCGGCGCCGAGAGATTCGTGCAGAGGACCGCTTC | |
| CTGCGCACAGAGCAGGGCTTGTTGCTCCGTGCACTG | |
| CAGCTCAGCGATCGTGGCCTCTACTCCTGCACAGCC | |
| ACTGAGAACAACTTTAAGCACGTCGTCACACGAGTG | |
| CAGCTGCATGTACTGGGCCGGGACGCCGTCCATGCT | |
| GCCCTCTTCCCACCACTGTCCATGAGCGCCCCGCCA | |
| CCCCCAGGCGCAGGCCCCCCAACGCCTCCTTACCAG | |
| GAGTTAGCCCAGCTGCTGGCCCAGCCAGAAGTGGGC | |
| CTCATCCACCAGTACTGCCAGGGTTACTGGCGCCAT | |
| GTGCCCCCCAGCCCCAGGGAGGCTCCAGGGGCACCC | |
| CGGTCTCCTGAGCCCCAGGACCAGAAAAAGCCCCGG | |
| AACCGCCGGCACCACCCTCCGGACACA | |
| CGGGGCCCAGGCCCCGCCGCTGCGGAAGAGGTTTCT | SEQ. ID NO. 10 |
| AGAGAGTGGAGCCTGCTTCCTGGGCCCTAGGCCCCT | |
| CCCACAATGCTTGTCGCCGGTCTTCTTCTCTGGGCT | |
| TCCCTACTGACCGGGGCCTGGCCATCCTTCCCCACC | |
| CAGGACCACCTCCCGGCCACGCCCCGGGTCCGGCTC | |
| TCATTCAAAGAGCTGAAGGCCACAGGCACCGCCCAC | |
| TTCTTCAACTTCCTGCTCAACACAACCGACTACCGA | |
| ATCTTGCTCAAGGACGAGGACCACGACCGCATGTAC | |
| GTGGGCAGCAAGGACTACGTGCTGTCCCTGGACCTG | |
| CACGACATCAACCGCGAGCCCCTCATTATACACTGG | |
| GCAGCCTCCCCACAGCGCATCGAGGAATGCGTGCTC | |
| TCAGGCAAGGATGTCAACGGCGAGTGTGGGAACTTC | |
| GTCAGGCTCATCCAGCCCTGGAACCGAACACACCTG | |
| TATGTGTGCGGGACAGGTGCCTACAACCCCATGTGC | |
| ACCTATGTGAACCGCGGACGCCGCGCCCAGGCCACA | |
| CCATGGACCCAGACTCAGGCGGTCAGAGGCCGCGGC | |
| AGCAGAGCCACGGATGGTGCCCTCCGCCCGATGCCC | |
| ACAGCCCCACGCCAGGATTACATCTTCTACCTGGAG | |
| CCTGAGCGACTCGAGTCAGGGAAGGGCAAGTGTCCG | |
| TACGATCCCAAGCTGGACACAGCATCGGCCCTCATC | |
| AATGAGGAGCTCTATGCTGGTGTGTACATCGATTTT | |
| ATGGGCACTGATGCAGCCATCTTCCGCACACTTGGA | |
| AAGCAGACAGCCATGCGCACGGATCAGTACAACTCC | |
| CGGTGGCTGAACGACCCGTCGTTCATCCATGCTGAG | |
| CTCATTCCTGACAGTGCGGAGCGCAATGATGATAAG | |
| CTTTACTTCTTCTTCCGTGAGCGGTCGGCAGAGGCG | |
| CCGCAGAGCCCCGCGGTGTACGCCCGCATCGGGCGC | |
| ATTTGCCTGAACGATGACGGTGGTCACTGTTGCCTG | |
| GTCAACAAGTGGAGCACATTCCTGAAGGCGCGGCTC | |
| GTCTGCTCTGTCCCGGGCGAGGATGGCATTGAGACT | |
| CACTTTGATGAGCTCCAGGACGTGTTTGTCCAGCAG | |
| ACCCAGGACGTGAGGAACCCTGTCATTTACGCTGTC | |
| TTTACCTCCTCTGGCTCCGTGTTCCGAGGCTCTGCC | |
| GTGTGTGTCTACTCCATGGCTGATATTCGCATGGTC | |
| TTCAACGGGCCCTTTGCCCACAAAGAGGGGCCCAAC | |
| TACCAGTGGATGCCCTTCTCAGGGAAGATGCCCTAC | |
| CCACGGCCGGGCACGTGCCCTGGTGGAACCTTCACG | |
| CCATCTATGAAGTCCACCAAGGATTATCCTGATGAG | |
| GTGATCAACTTCATGCGCAGCCACCCACTCATGTAC | |
| CAGGCCGTGTACCCTCTGCAGCGGCGGCCCCTGGTA | |
| GTCCGCACAGGTGCTCCCTACCGCCTTACCACTATT | |
| GCCGTGGACCAGGTGGATGCAGGCGACGGGCGCTAT | |
| GAGGTGCTTTTCCTGGGCACAGACCGCGGGACAGTG | |
| CAGAAGGTCATTGTGCTGCCCAAGGATGACCAGGAG | |
| ATGGAGGAGCTCATGCTGGAGGAGGTGGAGGTCTTC | |
| AAGGATCCAGCACCCGTCAAGACCATGACCATCTCT | |
| TCTAAGAGGCAACAACTCTACGTGGCGTCAGCCGTG | |
| GGTGTCACACACCTGAGCCTGCACCGCTGCCAGGCG | |
| TATGGGGCTGCCTGTGCTGACTGCTGCCTTGCCCGG | |
| GACCCTTACTGTGCCTGGGATGGCCAGGCCTGCTCC | |
| CGCTATACAGCATCCTCCAAGAGGCGGAGCCGCCGG | |
| CAGGACGTCCGGCACGGAAACCCCATCAGGCAGTGC | |
| CGTGGGTTCAACTCCAATGCCAACAAGAATGCCGTG | |
| GAGTCTGTGCAGTATGGCGTGGCCGGCAGCGCAGCC | |
| TTCCTTGAGTGCCAGCCCCGCTCGCCCCAAGCCACT | |
| GTTAAGTGGCTGTTCCAGCGAGATCCTGGTGACCGG | |
| CGCCGAGAGATTCGTGCAGAGGACCGCTTCCTGCGC | |
| ACAGAGCAGGGCTTGTTGCTCCGTGCACTGCAGCTC | |
| AGCGATCGTGGCCTCTACTCCTGCACAGCCACTGAG | |
| AACAACTTTAAGCACGTCGTCACACGAGTGCAGCTG | |
| CATGTACTGGGCCGGGACGCCGTCCATGCTGCCCTC | |
| TTCCCACCACTGTCCATGAGCGCCCCGCCACCCCCA | |
| GGCGCAGGCCCCCCAACGCCTCCTTACCAGGAGTTA | |
| GCCCAGCTGCTGGCCCAGCCAGAAGTGGGCCTCATC | |
| CACCAGTACTGCCAGGGTTACTGGCGCCATGTGCCC | |
| CCCAGCCCCAGGGAGGCTCCAGGGGCACCCCGGTCT | |
| CCTGAGCCCCAGGACCAGAAAAAGCCCCGGAACCGC | |
| CGGCACCACCCTCCGGACACATGAGGCCAGCTGCCT | |
| GTGCCTGCCATGGGCCAGGCTAGGCCTTGGTCCCTT | |
| TTAATATAAAAGATATATATATATATATATATATAT | |
| ATTAAAATATCGGGGTGGGGGGTGATTGGAAGGGAG | |
| GGAGGTGGCCTTCCCAATGCGCGTTATTCGGGGTTA | |
| TTGAAGAATAATATTGCAAGTGACAGCCAGAAGTAG | |
| ACTTTCTGTCCTCACACCGAAGAACCCGAGTGAGCA | |
| GGAGGGAGGGAGAGACGCGAAGAGACCTTTTTTCCT | |
| TTTTCGAGACCTTGTCCGC | |
| ATGCTTGTCCCCGGTCTTCTTCTCTGGGCTTCCCTA | SEQ. ID NO. 11 |
| CTGACCGGGGCCTGGCCATCCTTCCCCACCCAGGAC | |
| CACCTCCCGGCCACGCCCCGGGTCCGGCTCTCATTC | |
| AAAGACCTGAAGGCCACAGGCACCGCCCACTTCTTC | |
| AACTTCCTGCTCAACACAACCGACTACCGAATCTTG | |
| CTCAAGGACGAGGACCACGACCGCATGTACGTGGGC | |
| AGCAAGGACTACGTGCTCTCCCTGGACCTGCACGAC | |
| ATCAACCGCGAGCCCCTCATTATACACTGGGCAGCC | |
| TCCCCACAGCGCATCGAGGAATGCGTGCTCTCAGGC | |
| AAGGATGTCAACGGCGAGTGTGGGAACTTCGTCAGG | |
| CTCATCCAGCCCTGGAACCGAACACACCTGTATGTG | |
| TGCGGGACAGGTGCCTACAACCCCATGTGCACCTAT | |
| GTGAACCGCGGACGCCGCGCCCAGGCCACACCATGG | |
| ACCCAGACTCAGGCGGTCAGAGGCCGCGGCAGCAGA | |
| GCCACGGATGGTGCCCTCCGCCCGATGCCCACAGCC | |
| CCACGCCAGGATTACATCTTCTACCTGGAGCCTGAG | |
| CGACTCGAGTCAGGGAAGGGCAAGTGTCCGTACGAT | |
| CCCAAGCTGGACACAGCATCGGCCCTCATCAATGAG | |
| GAGCTCTATGCTGGTGTGTACATCGATTTTATGGGC | |
| ACTGATGCAGCCATCTTCCGCACACTTGGAAAGCAG | |
| ACAGCCATGCGCACGGATCAGTACAACTCCCGGTGG | |
| CTGAACGACCCGTCGTTCATCCATGCTGAGCTCATT | |
| CCTGACAGTGCGGAGCGCAATGATGATAAGCTTTAC | |
| TTCTTCTTCCGTGAGCGGTCGGCAGAGGCGCCGCAG | |
| AGCCCCGCGGTGTACGCCCGCATCGGGCGCATTTGC | |
| CTGAACGATGACGGTGGTCACTGTTGCCTGGTCAAC | |
| AAGTGGAGCACATTCCTGAAGGCGCGGCTCGTCTGC | |
| TCTGTCCCGGGCGAGGATGGCATTGAGACTCACTTT | |
| GATGAGCTCCAGGACGTGTTTGTCCAGCAGACCCAG | |
| GACGTGAGGAACCCTGTCATTTACGCTGTCTTTACC | |
| TCCTCTGGCTCCGTGTTCCGAGGCTCTGCCGTGTGT | |
| GTCTACTCCATGGCTGATATTCGCATGGTCTTCAAC | |
| GGGCCCTTTGCCCACAAAGAGGCGCCCAACTACCAG | |
| TGGATGCCCTTCTCAGGGAAGATGCCCTACCCACGG | |
| CCGGGCACGTGCCCTGGTGGAACCTTCACGCCATCT | |
| ATGAAGTCCACCAAGGATTATCCTGATGAGGTGATC | |
| AACTTCATGCGCAGCCACCCACTCATGTACCAGGCC | |
| GTGTACCCTCTGCAGCGGCGGCCCCTGGTAGTCCGC | |
| ACAGGTGCTCCCTACCGCCTTACCACTATTGCCGTG | |
| GACCAGGTGGATGCAGCCGACGGGCGCTATGAGGTG | |
| CTTTTCCTGGGCACAGACCGCGGGACAGTGCAGAAG | |
| GTCATTGTGCTGCCCAAGGATGACCAGGAGTTGGAG | |
| GAGCTCATGCTGGAGGAGGTGGAGGTCTTCAAGGAT | |
| CCAGCACCCGTCAAGACCATGACCATCTCTTCTAAG | |
| AGGCAACAACTCTACGTGGCGTCAGCCGTGGGTGTC | |
| ACACACCTGAGCCTGCACCGCTGCCAGGCGTATGGG | |
| GCTGCCTGTGCTGACTGCTGCCTTGCCCGGGACCCT | |
| TACTGTGCCTGGGATGGCCAGGCCTGCTCCCGCTAT | |
| ACAGCATCCTCCAAGAGGCGGAGCCGCCGGCAGGAC | |
| GTCCGGCACGGAAACCCCATCAGGCAGTGCCGTGGG | |
| TTCAACTCCAATGCCAACAAGAATGCCGTGGAGTCT | |
| GTGCAGTATGGCGTGGCCGGCAGCGCAGCCTTCCTT | |
| GAGTGCCAGCCCCGCTCGCCCCAAGCCACTGTTAAG | |
| TGGCTGTTCCAGCGAGATCCTGGTGACCGGCGCCGA | |
| GAGATTCGTGCAGAGGACCGCTTCCTGCGCACAGAG | |
| CAGGGCTTGTTGCTCCGTGCACTGCAGCTCAGCGAT | |
| CGTGGCCTCTACTCCTGCACAGCCACTGAGAACAAC | |
| TTTAAGCACGTCGTCACACGAGTGCAGCTGCATGTA | |
| CTGGGCCGGGACGCCGTCCATGCTGCCCTCTTCCCA | |
| CCACTGTCCATGAGCGCCCCGCCACCCCCAGGCGCA | |
| GGCCCCCCAACGCCTCCTTACCAGGAGTTAGCCCAG | |
| CTGCTGGCCCAGCCAGAAGTGGGCCTCATCCACCAG | |
| TACTGCCAGGGTTACTGGCGCCATGTGCCCCCCAGC | |
| CCCAGGGAGGCTCCAGGGGCACCCCGGTCTCCTGAG | |
| CCCCAGGACCAGAAAAAGCCCCGGAACCGCCGGCAC | |
| CACCCTCCGGACACA | |
| CCGCGGCGCCGATCCCGGCTGAGGCGCAGCGGCGAG | SEQ. ID NO. 12 |
| AGGTCGCGGGCAGGGCCATGGCCCCGGGGGGCCGCT | |
| AGCGCGGACCGGCCCAACGGGAGCCGCTCCGTGCCG | |
| CCGCCGCCGCCCGGGCGCCCAGGCCCCGCCGCTGCG | |
| GAAGAGGTTTCTAGAGAGTGGAGCCTGCTTCCTGGG | |
| CCCTAGGCCCCTCCCACAATGCTTGTCGCCGGTCTT | |
| CTTCTCTGGGCTTCCCTACTGACCGGGGCCTGGCCA | |
| TCCTTCCCCACCCAGGACCACCTCCCGGCCACGCCC | |
| CGGGTCCGGCTCTCATTCAAAGAGCTGAAGGCCACA | |
| GGCACCGCCCACTTCTTCAACTTCCTGCTCAACACA | |
| ACCGACTACCGAATCTTGCTCAAGGACGAGGACCAC | |
| GACCGCATGTACGTGGGCAGCAAGGACTACGTGCTG | |
| TCCCTGGACCTGCACGACATCAACCGCGAGCCCCTC | |
| ATTATACACTGGGCAGCCTCCCCACAGCGCATCGAG | |
| GAATGCGTGCTCTCAGGCAAGGATGTCAACGGCGAG | |
| TGTGGGAACTTCGTCAGGCTCATCCAGCCCTGGAAC | |
| CGAACACACCTGTATGTGTGCGGGACAGGTGCCTAC | |
| AACCCCATGTGCACCTATGTGAACCGCGGACGCCGC | |
| GCCCAGGCCACACCATGGACCCAGACTCAGGCGGTC | |
| AGAGGCCGCGGCAGCAGAGCCACGGATGGTGCCCTC | |
| CGCCCGATGCCCACAGCCCCACGCCAGGATTACATC | |
| TTCTACCTGGAGCCTGAGCGACTCGAGTCAGGGAAG | |
| GGCAAGTGTCCGTACGATCCCAAGCTGGACACAGCA | |
| TCGGCCCTCATCAATGAGGAGCTCTATGCTGGTGTG | |
| TACATCGATTTTATGGGCACTGATGCAGCCATCTTC | |
| CGCACACTTGGAAAGCAGACAGCCATGCGCACGGAT | |
| CAGTACAACTCCCGGTGGCTGAACGACCCGTCGTTC | |
| ATCCATGCTGAGCTCATTCCTGACAGTGCGGAGCGC | |
| AATGATGATAAGCTTTACTTCTTCTTCCGTGAGCGG | |
| TCGGCAGAGGCGCCGCAGAGCCCCGCGGTGTACGCC | |
| CGCATCGGGCGCATTTGCCTGAACGATGACGGTGGT | |
| CACTGTTGCCTGGTCAACAAGTGGAGCACATTCCTG | |
| AAGGCGCGGCTCGTCTGCTCTGTCCCGGGCGAGGAT | |
| GGCATTGAGACTCACTTTGATGAGCTCCAGGACGTG | |
| TTTGTCCAGCAGACCCAGGACGTGAGGAACCCTGTC | |
| ATTTACGCTGTCTTTACCTCCTCTGGCTCCGTGTTC | |
| CGAGGCTCTGCCGTGTGTGTCTACTCCATGGCTGAT | |
| ATTCGCATGGTCTTCAACGGGCCCTTTGCCCACAAA | |
| GAGGGGCCCAACTACCAGTGGATGCCCTTCTCAGGG | |
| AAGATGCCCTACCCACGGCCGGGCACGTGCCCTGGT | |
| GGAACCTTCACGCCATCTATGAAGTCCACCAAGGAT | |
| TATCCTGATGAGGTGATCAACTTCATGCGCAGCCAC | |
| CCACTCATGTACCAGGCCGTGTACCCTCTGCAGCGG | |
| CGGCCCCTGGTAGTCCGCACAGGTGCTCCCTACCGC | |
| CTTACCACTATTGCCGTGGACCAGGTGGATGCAGCC | |
| GACGGGCGCTATGAGGTGCTTTTCCTGGGCACAGAC | |
| CGCGGGACAGTGCAGAAGGTCATTGTGCTGCCCAAG | |
| GATGACCAGGAGATGGAGGAGCTCATGCTGGAGGAG | |
| GTGGAGGTCTTCAAGGATCCAGCACCCGTCAAGACC | |
| ATGACCATCTCTTCTAAGAGGCAACAACTCTACGTG | |
| GCGTCAGCCGTGGGTGTCACACACCTGAGCCTGCAC | |
| CGCTGCCAGGCGTATGGGGCTGCCTGTGCTGACTGC | |
| TGCCTTGCCCGGGACCCTTACTGTGCCTGGGATGGC | |
| CAGGCCTGCTCCCGCTATACAGCATCCTCCAAGAGG | |
| CGGAGCCGCCGGCAGGACGTCCGGCACGGAAACCCC | |
| ATCAGGCAGTGCCGTGGGTTCAACTCCAATGCCAAC | |
| AAGAATGCCGTGGAGTCTGTGCAGTATGGCGTGGCC | |
| GGCAGCGCAGCCTTCCTTGAGTGCGAGCCCCGCTCG | |
| CCCCAAGCCACTGTTAAGTGGCTGTTCCAGCGAGAT | |
| CCTGGTGACCGGCGCCGAGAGATTCGTGCAGAGGAC | |
| CGCTTCCTGCGCACAGAGCAGGGCTTGTTGCTCCGT | |
| GCACTGCAGCTCAGCGATCGTGGCCTCTACTCCTGC | |
| ACAGCCACTGAGAACAACTTTAAGCACGTCGTCACA | |
| CGAGTGCAGCTGCATGTACTGGGCCGGGACGCCGTC | |
| CATGCTGCCCTCTTCCCACCACTGTCCATGAGCGCC | |
| CCGCCACCCCCAGGCGCAGGCCCCCCAACGCCTCCT | |
| TACCAGGAGTTAGCCCAGCTGCTGGCCCAGCCAGAA | |
| GTGGGCCTCATCCACCAGTACTGCCAGGGTTACTGG | |
| CGCCATGTGCCCCCCAGCCCCAGGGAGGCTCCAGGG | |
| GCACCCCGGTCTCCTGAGCCCCAGGACCAGAAAAAG | |
| CCCCGGAACCGCCGGCACCACCCTCCGGACACATGA | |
| GGCCAGCTGCCTGTGCCTGCCATGGGCCAGCCTAGC | |
| CCTTGTCCCTTTTAATATAAAAGATATATATATATA | |
| TATATATATATATATAAAATATCTATATTCTATACA | |
| CACCCTGCCCCTGCAAAGACAGTATTTATTGGTGGG | |
| TTGAATATAGCCTGCCTCAGTGGCAGCATCCTCCAA | |
| AACTTAGACCCATGCTGGTCAGAGACGGCAGAAAAC | |
| AGAGCCTGCCTAACCAGGCCCAGCCAGTTGGTGGGG | |
| CCAGGCCAGGACCACACAGTCCCCAGACTCAGCTGG | |
| AAGTCTACCTGCTGGACAGCCTCCGCCAAGATCTAC | |
| AGGACAAAGGGAGGGAGCAAGCCCTACTCGGATGGG | |
| GCACGGACTGTCCACCTTTTCTGATGTGTGTTGTCA | |
| GCCTGTGCTGTGGCATAGACATGGATGCGAGGACCA | |
| CTTTGGAGACTGGGGTGGCCTCAAGAGCACACAGAG | |
| AAGGGAAGAAGGGGCCATCACAGGATGCCA | |

1. A method of treating or preventing a disease in a subject comprising:
(a) providing semaphorin 3F or a biologically active variant or fragment thereof and a pharmaceutically acceptable carrier; and
(b) administering the semaphorin 3F to the subject;
wherein the subject benefits from a response of the semaphorin 3F receptors on muscle cells.

2. A method of treating or preventing a disease in a subject comprising:
(a) providing an inhibitor or antagonist of semaphorin 3F and a pharmaceutically acceptable carrier; and
(b) administering the inhibitor or antagonist to the subject;
wherein the subject benefits from blocking a response of the semaphorin 3F receptors on muscle cells.

3. The method of claim 1 or claim 2, wherein the disease is a muscular disease.

4. The method of claim 3, wherein the muscular disease is a neuromuscular disease.

5. The method of claim 3, wherein the muscular disease comprises muscular atrophy.

6. The method of claim 5, wherein the atrophy results from denervation.

7. The method of claim 6, wherein the denervation results from a condition chosen from acute injury, diabetic neuropathy, Charcot-Marie-Tooth disorder, chronic demyelinating polyradiculoneuropathy, toxin-induced neuropathy, amyotrophic lateral sclerosis, poliomyelitis, post-polio syndrome, nerve entrapment syndromes, and HIV-associated neuropathy.

8. The method of claim 1 or claim 2, wherein the subject is a human or non-human animal.

9. The method of claim 1 or claim 2, wherein the semaphorin 3F is chosen from the group of SEQ. ID. NOs.:5-8, and active variants or fragments of any of these.

10. The method of claim 1 or claim 2, wherein the muscle cells are skeletal muscle cells.

11. A diagnostic kit comprising a composition that comprises a semaphorin 3F nucleic acid molecule, a reporter for detecting the nucleic acid molecule and/or its complement, and instructions for their use in diagnosing a muscular disease.

12. A diagnostic kit comprising an antibody that specifically binds to a semaphorin 3F polypeptide, a carrier, a reporter for detecting the antibody binding to the polypeptide, and instructions for their use in diagnosing a muscular disease.

13. A method of determining the presence of an antibody specific to a semaphorin 3F polypeptide in a patient sample comprising:
(a) providing a composition comprising a semaphorin 3F polypeptide;
(b) allowing the polypeptide to interact with the sample; and
(c) determining whether interaction has occurred between the polypeptide and antibody in the sample, if present.

14. An isolated antibody that specifically binds to and/or interferes with the activity of an antigen in the vicinity of a muscle cell that comprises at least six contiguous amino acid residues derived from a sequence selected from the group of SEQ. ID. NOs.:5-8.

15. An isolated antibody that specifically binds to and/or interferes with the activity of an antigen in the vicinity of a muscle cell that is encoded by a polynucleotide comprising at least eighteen contiguous nucleotides derived from a sequence selected from the group of SEQ. ID. NOs.:1-4.

16. The antibody of claim 14 or claim 15, wherein the antibody comprises a polyclonal antibody, a monoclonal antibody, a single chain antibody, or active fragments of any of these.

17. The antibody of claim 16, wherein the active fragment comprises an antigen binding fragment, an Fc fragment, a cdr fragment, a V_{H} fragment, a V_{C} fragment, or a framework fragment.

18. A method of screening for an agent that modulates the activity of a semaphorin 3F polypeptide comprising:
(a) providing a test system in which semaphorin 3F polypeptide affects biological activity; and
(b) screening multiple agents for an effect on the activity of the polypeptide on the test system,
wherein the effect comprises inhibition or stimulation of muscle cell activity.

19. The method of claim 18, wherein the agent is chosen from a small molecule drug, an antibody, a soluble semaphorin 3F receptor or receptor fragment, and a semaphorin 3F polypeptide variant or fragment.

20. A method of modulating a muscle cell activity comprising:
(a) providing a composition comprising a substantially pure semaphorin 3F polypeptide or a fragment or a variant thereof; and
(b) contacting one or more muscle cells with the composition.

21. A method of modulating a muscle cell activity comprising:
(a) providing a composition comprising a vector comprising a semaphorin 3F nucleic acid molecule or fragment thereof; and
(b) contacting one or more muscle cells with the composition.

22. A method of modulating a muscle cell activity comprising:
(a) providing a composition comprising an inhibitor or antagonist of semaphorin 3F; and
(b) contacting one or more muscle cells with the composition.

23. The method of any of claims 20-22, wherein the modulation of muscle cell activity affects the formation or maintenance of neuromuscular junctions.

24. The method of any of claims 20-22, wherein the modulation of muscle cell activity contributes to neuromuscular regeneration.

25. The method of any of claims 20-22, wherein the muscle cell is a skeletal muscle cell.

26. A method of modulating the formation and/or maintenance of neuromuscular junctions in a subject comprising:
(a) providing a composition comprising a substantially pure semaphorin 3F polypeptide; and
(b) administering the composition to the subject.

27. A method of modulating the formation and/or maintenance of neuromuscular junctions in a subject comprising:
(a) providing a composition comprising a vector comprising a semaphorin 3F nucleic acid molecule or fragment thereof; and
(b) administering the composition to the subject.

28. A method of modulating the formation and/or maintenance of neuromuscular junctions in a subject comprising:
(a) providing a composition comprising an inhibitor or antagonist of semaphorin 3F; and
(b) administering the composition to the subject.

29. The method of any of claim 2, claim 22, or claim 28, wherein the inhibitor or antagonist is selected from semaphorin 3F or a variant or fragment thereof; an antibody directed to semaphorin 3F; an antibody directed to a semaphorin 3F receptor; a soluble semaphorin 3F receptor or fragment thereof; a small molecule drug; an RNAi molecule; an antisense molecule; a peptide; an aptamer; and a ribozyme that inhibits semaphorin 3F expression and/or activity.

30. The method of any of claims 26-29, wherein the modulation of the formation and/or maintenance of neuromuscular junctions contributes to neuromuscular regeneration.

31. The method of any of claims 26-29, wherein the modulation of the formation and/or maintenance of neuromuscular junctions contributes to treatment or prevention of a neuromuscular disease.

32. The method of any of claims 26-31, wherein the composition is administered to the subject locally or systemically.

33. The method of claim 32, wherein the composition is administered intravenously, by enema, intraperitoneally, subcutaneously, topically, or transdermally.
